(19) United States
(12) Patent Application Publication
(10) Pub. No.: US 2026/0167956 A1
(43) Pub. Date: Jun. 18, 2026

(54) NON-VIRAL DELIVERY COMPOSITIONS AND SCREENING METHODS

(71) Applicant: BATTELLE MEMORIAL INSTITUTE, Columbus, OH (US)

(72) Inventors: Cherry Gupta, Columbus, OH (US); Anthony D. Duong, Columbus, OH (US); Danielle J. Huk, Hilliard, OH (US); Kenneth R. Sims, Jr., Delaware, OH (US); Michael S. Koeris, Columbus, OH (US); Miguel D. Pedrozo, Columbus, OH (US); Nickolas R. Andrioff, Columbus, OH (US); Zachary R. Shank, London, OH (US); Ashlee J. Colbert, Blacklick, OH (US); Andrea D. McCue, Columbus, OH (US); Emma K. Schmitz, Columbus, OH (US); Caleb T. Hillrich, Columbus, OH (US); Shannon D. Miller, Hilliard, OH (US); Joanna L. Hoy, Columbus, OH (US); Natalie Hoffman, Westerville, OH (US)

(21) Appl. No.: 19/355,850

(22) Filed: Oct. 10, 2025

Related U.S. Application Data
(63) Continuation of application No. 18/332,065, filed on Jun. 9, 2023, now abandoned.
(60) Provisional application No. 63/350,688, filed on Jun. 9, 2022.

(51) Int. Cl. C12N 15/10; A61K 48/00

(52) U.S. Cl. CPC C12N 15/1093; A61K 48/005

(57) ABSTRACT

The invention relates to barcoded nucleic acid nanostructure delivery compositions for in vivo screening for subsequent use in vivo therapeutic delivery, and methods therefor. More particularly, the invention relates to nucleic acid nanostructure delivery compositions, such as DNA origami structures, associated with barcodes for high throughput in vivo screening of the nucleic acid nanostructure delivery compositions for subsequent use in drug delivery, and methods therefor.

### CROSS-REFERENCE TO RELATED APPLICATIONS

This application is a continuation of U.S. application Ser. No. 18/332,065, filed on Jun. 9, 2023, which claims the benefit and priority, under 35 U.S.C. § 119(e) and any other applicable laws and statutes, to U.S. Provisional Application Ser. No. 63/350,688 filed on Jun. 9, 2022, the entire disclosure of which is incorporated herein by reference.

### INCORPORATION BY REFERENCES OF MATERIAL SUBMITTED ELECTRONICALLY

Incorporated by reference in its entirety is a computer-readable nucleotide/amino acid sequence listing submitted concurrently herewith and identified as follows: 1,055,000 byte xml file named “920006-391462_SL.xml,” created on Sep. 7, 2023.

### FIELD OF THE DISCLOSURE

The invention relates to barcoded nucleic acid nanostructure delivery compositions for in vivo screening for subsequent use in vivo therapeutic delivery, and methods therefor. More particularly, the invention relates to nucleic acid nanostructure delivery compositions, such as DNA origami structures, associated with barcodes for high throughput in vivo screening of the nucleic acid nanostructure delivery compositions for subsequent use in drug delivery, and methods therefor.

### BACKGROUND AND SUMMARY

Genetic medicines (including gene therapy, gene silencing, splicing regulators, and nuclease based gene editors) are poised to produce revolutionary treatments, including vaccines, infectious disease treatments, antimicrobial treatments, antiviral treatments, and most notably, genetic disease treatments. However, the in vivo delivery of these genetic medicine payloads to the specific tissues and cells that need to be treated, while avoiding tissues and cells that can reduce the efficacy or safety of the genetic medicine, poses a significant challenge. Additional challenges include the ability to deliver large genetic payloads or multiple payloads. Adeno-associated viruses (AAVs) are the most widely used tool for genetic medicine delivery, but AAVs are not able to deliver large genetic payloads or multiple payloads (such as the clustered regularly interspaced short palindromic repeats (CRISPR)/Cas9 system), and they sometimes trigger unwanted immune responses, including the generation of anti-AAV antibodies, a cell mediated response. Some of the immune responses caused by AAV in patients are potentially fatal immune responses.

Therapeutics based on the CRISPR/Cas9 system have an exceptional potential to treat a number of genetic diseases due to the capability of this system for precise and programmable gene editing. Gene editing and repair using the CRISPR/Cas9 system has two main mechanisms, including non-homologous end joining (NHEJ) which repairs the site of cut by inducing random indel mutation, and homology-directed repair (HDR), which repairs the cut site based on a pre-existing template. Because a pre-designed template can be used for HDR-directed repair, therapies based on this mechanism can be tailored to cure a large number of different genetic diseases. However, the main challenge is that HDR repair requires the delivery of CRISPR/Cas9, small guide RNA (sgRNA) and a donor DNA strand at the same time to a particular location. This requirement becomes particularly limiting for in vivo applications because ensuring co-delivery of multiple large molecules to the same targeted location is currently not feasible. For example, the Cas9 enzyme sequence and guide RNA complex is too large to fit into AAVs.

There is a need for effective non-viral delivery systems, not only for genetic delivery systems, but also for delivery of small molecule therapeutics. The current state-of-the-art non-viral gene delivery systems, such as liposomes, have many drawbacks such as poor biocompatibility and the inability to easily engineer or functionalize them. Additional concerns are that such non-viral gene delivery systems are easily degraded by various enzymes as they pass through intracellular or intercellular compartments, and these systems have not been able to package multiple large payloads.

The inventors have designed nucleic acid nanostructure delivery compositions (e.g., DNA origami nanostructures). These compositions have the advantage of being biocompatible, non-toxic, and can be programmed in many ways. For example, the nucleic acid nanostructure delivery compositions can be programmed to have functional groups that enable them to evade early degradation, that enable them to evade immune responses, and that enable intracellular imaging and targeted and controlled delivery of therapeutic genes and small molecule therapeutics. Thus, these non-viral delivery compositions can enhance the stability, safety, and/or efficacy of payloads by providing immune evasion, tissue-directed intracellular delivery, and the ability to deliver large genetic payloads or multiple payloads, or other genetic medicine payloads, or small molecule therapeutics.

The rate limiting step for development of such drug delivery vehicles is in vivo testing of the drug delivery vehicles leading to slow or un-optimized final products and a lack of new drug delivery vehicle candidates. The inventors have demonstrated the utility of a nucleic acid nanostructure delivery composition (e.g., a DNA origami nanostructure composition) for in vivo delivery, and the inventors have also developed novel methods for labeling the nucleic acid nanostructure delivery compositions with unique barcodes, administering them to an animal, and then extracting them from animal tissues for detection. This method will allow for in vivo high throughput screening of a diverse set of drug delivery nanoparticles, including DNA origami structures, for use in the delivery of large genetic payloads, multiple payloads, other genetic medicine payloads, or small molecule therapeutics.

The following clauses, and combinations thereof, provide various additional illustrative aspects of the invention described herein. The various embodiments described in any other section of this patent application, including the section titled “DETAILED DESCRIPTION OF ILLUSTRATIVE EMBODIMENTS” and the “EXAMPLES” are applicable to any of the following embodiments of the invention described in the numbered clauses below.
1. A composition comprising a non-viral delivery vehicle comprising a nucleic acid nanostructure delivery composition, and a nucleic acid barcode construct.
2. The composition of clause 1, wherein the nucleic acid nanostructure delivery composition comprises a DNA origami composition.
3. The composition of clause 1, wherein the nucleic acid nanostructure delivery composition comprises single-stranded or double-stranded DNA or RNA.
4. The composition of any one of clauses 1 to 3, wherein the nucleic acid barcode construct is associated with the nucleic acid nanostructure delivery composition via base-pairing.
5. The composition of clause 4, wherein the base-pairing occurs between a sequence of a single-stranded overhang on the nucleic acid nanostructure delivery composition and a complementary sequence appended to the nucleic acid barcode construct.
6. The composition of any one of clauses 1 to 3, wherein the nucleic acid nanostructure delivery composition comprises staples that self-assemble to form the nucleic acid nanostructure delivery composition.
7. The composition of clause 6, wherein the staples act as the nucleic acid barcode construct.
8. The composition of any one of clauses 1 to 3, wherein the nucleic acid barcode construct is associated with the nucleic acid nanostructure delivery composition by a high affinity, non-covalent bond interaction between a biotin molecule on the 5′ and/or the 3′ end of the nucleic acid barcode construct and a molecule that binds to biotin on the nucleic acid nanostructure delivery composition.
9. The composition of clause 8, wherein the molecule that binds to biotin is bound to the nucleic acid nanostructure delivery composition by a covalent phosphoramidate bond formed via an EDC-NHS coupling reaction between a terminal phosphate group of a 5′ end of an overhang on the nucleic acid nanostructure delivery composition and an amine group on the molecule that binds to biotin.
10. The composition of clause 8 or 9, wherein the biotin is bound to the nucleic acid barcode construct by a covalent bond.
11. The composition of any one of clauses 1 to 3, wherein the nucleic acid barcode construct is bound to the nucleic acid nanostructure delivery composition by a covalent bond.
12. The composition of clause 11, wherein the covalent bond is formed via an EDC-NHS coupling reaction between a terminal phosphate group of the 5′ end of an overhang on the nucleic acid nanostructure delivery composition and an amine group on an amino terminal nucleotide of the nucleic acid barcode construct.
13. The composition of clause 11, wherein the covalent bond is formed via a click chemistry coupling reaction between an azide group on the nucleic acid nanostructure delivery composition and an alkyne group on the nucleic acid barcode construct.
14. The composition of clause 11, wherein the covalent bond is formed via a click chemistry coupling reaction between an azide group on the nucleic acid barcode construct and an alkyne group on the nucleic acid nanostructure delivery composition.
15. The composition of any one of clauses 1 to 3, wherein the nucleic acid barcode construct is associated with the nucleic acid nanostructure delivery composition by a covalent bond between a carboxy terminated molecule on the nucleic acid nanostructure delivery composition and a primary amine on the nucleic acid barcode construct at the 5′ and/or the 3′ end.
16. The composition of any one of the preceding clauses, wherein the nucleic acid barcode construct comprises two primer binding segments and one or more unique barcode sequences between the two primer binding segments.
17. The composition of clause 16, wherein the primer binding segments range in length from about 15 base pairs to about 30 base pairs.
18. The composition of clause 16 or 17, wherein the primer binding segments are a universal primer binding set.
19. The composition of any one of clauses 16 to 18, wherein the one or more unique barcode sequences comprise unique sequences of about 6 to about 20 nucleotides in length.
20. The composition of any one of clauses 16 to 19, wherein the length of the unique barcode sequences is two times or more greater than the length of the primer binding segments.
21. The composition of any one of clauses 1 to 20, wherein the nucleic acid barcode construct comprises DNA or RNA.
22. The composition of any one of clauses 16 to 21, wherein the unique barcode sequences further comprise a hamming distance of at least 2 to 6 bases between any two unique barcode sequences.
23. The composition of any one of the preceding clauses, wherein the nucleic acid barcode construct further comprises from about 6 to about 12 random bases at the 3′ end of the unique barcode sequences.
24. The composition of clause 23, wherein the about 6 to about 12 random bases at the 3′ end of the unique barcode sequences are for bioinformatic removal of PCR duplicates.
25. The composition of any one of the preceding clauses, wherein the nucleic acid barcode construct ranges in length from about 42 nucleotides to about 210 nucleotides.
26. A method of in vivo screening for a desired nucleic acid nanostructure delivery composition, the method comprising (a) preparing a library comprising two or more types of nucleic acid nanostructure delivery compositions, wherein each nucleic acid nanostructure delivery composition is associated with a nucleic acid barcode construct comprising a different unique barcode sequence, (b) administering the library to an animal, (c) removing cells or tissues from the animal, (d) isolating the nucleic acid barcode constructs from the cells or the tissues of the animal, (e) detecting the nucleic acid barcode constructs in the cells or the tissues of the animal, and (f) identifying the desired nucleic acid nanostructure delivery composition for use as a delivery vehicle.
27. The method of clause 26, wherein the nucleic acid nanostructure delivery composition is associated with the nucleic acid barcode construct according to any one of clauses 4 to 15.
28. The method of clause 26 or 27, wherein the nucleic acid barcode construct is detected by a method selected from the group consisting of the polymerase chain reaction (PCR), isothermal amplification, sequencing, or a combination thereof, to obtain nucleotide sequence data.
29. The method of any one of clauses 26 to 28, wherein the nucleic acid nanostructure delivery composition is loaded with a payload.
30. The method of clause 29, wherein the payload is a luminescent molecule.
31. The method of clause 30, wherein the luminescence is used to track the biodistribution or cell uptake of the nucleic acid nanostructure delivery composition via imaging.
32. The method of any one of clauses 26 to 31, wherein the administration to the animal is via an intramuscular, an intravenous, an intraperitoneal, an oral, or a pulmonary route.
33. The method of any one of clauses 26 to 32, wherein the nucleic acid barcode construct is isolated from the cells and the tissues by mixing with a first organic compound and incubating the organic phase with an aqueous phase of the cell or tissue sample, separating the organic phase from the aqueous phase, mixing the organic phase with a second organic compound, incubating the mixture, precipitating the nucleic acid barcode construct from the mixture, removing the organic phase by evaporation, and resuspending the nucleic acid barcode construct in an aqueous composition.
34 The method of clause 33, wherein the organic phase comprises phenol chloroform.
35. The method of clause 26, wherein the nucleic acid barcode construct is separated from cationic material in the cells or tissues by titrating the aqueous composition of the nucleic acid barcode construct to a pH of greater than 7.4.
36. The method of clause 26, wherein the nucleic acid barcode construct is separated from material in the cells or tissues by binding the nucleic acid barcode construct with a molecule with a binding affinity to the nucleic acid barcode construct greater than the binding affinity to the cell or tissue material.
37. The method of clause 26, wherein the nucleic acid barcode construct is separated from material in the cells or tissues via size exclusion chromatography.
38. The method of clause 26, wherein the nucleic acid barcode construct is separated from material in the cells or tissues via dialysis or diafiltration.
39. The method of clause 26, wherein the nucleic acid barcode construct is separated from material in the cells or tissues via filtration.
40. The method of clause 26, wherein the nucleic acid barcode construct is separated from material in the cells or tissues by digesting proteins using an enzyme.
41. The method of clause 40, wherein the enzyme is Proteinase K.
42. The method of clause 26, wherein the nucleic acid barcode constructs associated with the nucleic acid nanostructure delivery composition are detected by first diluting the isolated nucleic acid barcode constructs by a factor of at least 1000 times, and then amplifying the nucleic acid barcode constructs by PCR using primers.
43. The method of clause 42, wherein the primers from the PCR step are enzymatically digested prior to detection of amplicons.
44. The method of clause 28, wherein the nucleotide sequence data is converted to fast Q files, and the fast Q files are mapped to known unique polynucleotide sequences and the unique polynucleotide sequences are enumerated.
45. The method of any one of clauses 26 to 44, wherein the nucleic acid barcode construct of any one clauses 16 to 25 is used.
46. A composition comprising a non-viral delivery vehicle comprising a nucleic acid nanostructure delivery composition and a payload wherein the nucleic acid nanostructure delivery composition comprises single-stranded or double-stranded DNA or RNA.
47. The composition of clause 46, wherein the nucleic acid nanostructure delivery composition comprises DNA.
48. The composition of clause 46, wherein the nucleic acid nanostructure delivery composition comprises RNA.
49. The composition of clause 46, wherein the nucleic acid nanostructure delivery composition is single-stranded.
50. The composition of clause 46, wherein the nucleic acid nanostructure delivery composition is double-stranded.
51. The composition of any one of clauses 46 to 50, wherein the payload comprises nucleic acids.
52. The composition of clause 51, wherein the nucleic acids comprise DNA or RNA.
53. The composition of clause 51, wherein the payload nucleic acids are used for homology directed repair or as transposable elements.
54 The composition of clause 51, wherein the payload nucleic acids comprise a short guide RNA (sgRNA) and a donor DNA strand.
55. The composition of clause 54, wherein the sgRNA is used for targeting an enzyme to a specific genomic sequence.
56. The composition of any one of clauses 46 to 50, wherein the payload comprises a CRISPR associated enzyme.
57. The composition of clause 55, wherein the targeted enzyme is a CRISPR associated enzyme.
58. The composition of clause 51, wherein the payload comprises a CRISPR associated enzyme, an sgRNA, and a donor DNA strand.
59. The composition of any one of clauses 46 to 50, wherein the payload comprises CRISPR/Cas9.
60. The composition of clause 51, wherein the payloads comprise CRISPR/Cas9, an sgRNA, and a donor DNA strand.
61. The composition of any one of clauses 46 to 50, wherein the payload comprises CRISPR/Cas9 and Cas9 is fused with a deaminase.
62. The composition of clause 51, wherein the payloads comprise a coding sequence for Cas9, an sgRNA, and a donor DNA strand in the form of a plasmid.
63. The composition of clause 51, wherein the payloads consist of one molecule each of CRISPR/Cas9, an sgRNA, and a donor DNA strand.
64. The composition of clause 51, wherein the payloads comprise an antisense oligonucleotide.
65. The composition of clause 51, wherein the payload is of a size selected from the group consisting of 3 kB or more, 3.5 kB or more, 4 kB or more, 4.5 kB or more, 5 kB or more, 5.5 kB or more, 6 kB or more, 6.5 kB or more, 7 kB or more, 7.5 kB or more, 8 kB or more, and 8.5 kB or more.
66. The composition of any one of clauses 46 to 51, wherein the nucleic acid nanostructure delivery composition comprises one or more oligonucleotides with overhangs that bind through complementary base paring with the payload nucleic acids.
67. The composition of any one of clauses 46 to 65, wherein the payload is associated with the nucleic acid nanostructure delivery composition by a high affinity, non-covalent bond interaction between a biotin molecule on the payload and a molecule that binds to biotin on the nucleic acid nanostructure delivery composition.
68. The composition of clause 67, wherein molecule that binds to biotin is bound to the nucleic acid nanostructure delivery composition by a covalent phosphoramidate bond formed via an EDC-NHS coupling reaction between a terminal phosphate group of a 5′ end of an overhang on the nucleic acid nanostructure delivery composition and an amine group on the molecule that binds to biotin.
69 The composition of clause 67 or 68, wherein the biotin is bound to the payload by a covalent bond.
70 The composition of any one of clauses 46 to 65, wherein the payload is bound to the nucleic acid nanostructure delivery composition by a covalent bond.
71. The composition of clause 70, wherein the covalent bond is formed via an EDC-NHS coupling reaction between a terminal phosphate group of the 5′ end of an overhang on the nucleic acid nanostructure delivery composition and an amine group on the payload.
72. The composition of clause 70, wherein the covalent bond is formed via a click chemistry coupling reaction between an azide group on the nucleic acid nanostructure delivery composition and an alkyne group on the payload.
73. The composition of clause 70, wherein the covalent bond is formed via a click chemistry coupling reaction between an azide group on the payload and an alkyne group on the nucleic acid nanostructure delivery composition.
74. The composition of any one of clauses 46 to 65, wherein the payload is associated with the nucleic acid nanostructure delivery composition by a covalent bond between a carboxy terminated molecule on the nucleic acid nanostructure delivery composition and an amine on the payload.
75. The composition of any one of clauses 46 to 74, wherein the nucleic acid nanostructure delivery composition has an aspect ratio of about 2.
76. The composition of any one of clauses 1 to 25 or 46 to 75, wherein the nucleic acid nanostructure delivery composition is coated with one or more polymers.
77. The composition of any one of clauses 1 to 25 or 46 to 76, wherein the nucleic acid nanostructure delivery composition further comprises a targeting component for targeting to cells.
78. A method of treating a patient with a disease, the method comprising administering to the patient the nucleic acid nanostructure delivery composition identified in the in vivo screening method of any one of clauses 26 to 45 or the nucleic acid nanostructure delivery composition of any one of clauses 46 to 77, wherein the nucleic acid nanostructure delivery composition comprises a payload, and treating the disease in the patient.
79. The method of clause 78, further comprising administering a pharmaceutically acceptable carrier to the patient.
80. The method of clause 79, wherein the pharmaceutically acceptable carrier is for parenteral administration or topical administration.
81. The method of clause 78, wherein the patient has a disease or a disorder selected from the group consisting of cancer, a muscular disorder, a pulmonary disorder, a skin disorder, a neurological disease, neurofibromatosis 1, and a hemoglobinopathy.
82 The method of clause 81, wherein the cancer is selected from the group consisting of lung cancer, bone cancer, pancreatic cancer, skin cancer, uterine cancer, ovarian cancer, endometrial cancer, rectal cancer, stomach cancer, colon cancer, breast cancer, cancer of the esophagus, cancer of the endocrine system, prostate cancer, leukemia, lymphoma, mesothelioma, cancer of the bladder, cancer of the kidney, neoplasms of the central nervous system, brain cancer, and adenocarcinoma.
83. The method of clause 81, wherein the skin disorder is a Staphylococcus aureus infection.
84 The method of clause 81, wherein the muscular disorder is muscular dystrophy.
85. The method of clause 78, wherein the nucleic acid nanostructure delivery composition is not cytotoxic to the cells of the patient.

### BRIEF DESCRIPTION OF THE DRAWINGS

FIG. 1 TEM image of a DNA barcoded origami structure with 1% PTA negative stain at 100,000× magnification. The white rectangular structures are the DNA origami structures.

FIG. 2 shows the gel electrophoresis image of the PCR amplification products for the various test articles and controls. The presence of the white bands indicates the presence of PCR amplicons consistent with amplification of the DNA barcodes.

FIG. 3 shows PCR amplification of DNAO with and without barcodes at various transfection concentrations.

### DETAILED DESCRIPTION OF ILLUSTRATIVE EMBODIMENTS

The invention relates to barcoded nucleic acid nanostructure delivery compositions for in vivo screening for subsequent use in vivo therapeutic delivery, and methods therefor. More particularly, the invention relates to nucleic acid nanostructure delivery compositions, such as DNA origami structures, associated with barcodes for high throughput in vivo screening of the nucleic acid nanostructure delivery compositions for subsequent use of the nucleic acid nanostructure delivery compositions in drug delivery, and methods therefor.

The invention also relates to nucleic acid nanostructure delivery compositions for non-viral delivery, and methods therefor. More particularly, the invention relates to single-stranded or double-stranded DNA or RNA nanostructure delivery compositions, such as DNA origami compositions, for the delivery of more than one payload, a nucleic acid construct payload of 3 kB or more, other genetic medicine payloads, or small molecule therapeutics.

The following clauses, and combinations thereof, provide various additional illustrative aspects of the invention described herein. The various embodiments described in any other section of this patent application, including the summary portion of the section titled “BACKGROUND AND SUMMARY”, the “EXAMPLES”, and this “DETAILED DESCRIPTION OF ILLUSTRATIVE EMBODIMENTS” section of the application are applicable to any of the following embodiments of the invention described in the numbered clauses below.
1. A composition comprising a non-viral delivery vehicle comprising a nucleic acid nanostructure delivery composition, and a nucleic acid barcode construct.
2. The composition of clause 1, wherein the nucleic acid nanostructure delivery composition comprises a DNA origami composition.
3. The composition of clause 1, wherein the nucleic acid nanostructure delivery composition comprises single-stranded or double-stranded DNA or RNA.
4. The composition of any one of clauses 1 to 3, wherein the nucleic acid barcode construct is associated with the nucleic acid nanostructure delivery composition via base-pairing.
5. The composition of clause 4, wherein the base-pairing occurs between a sequence of a single-stranded overhang on the nucleic acid nanostructure delivery composition and a complementary sequence appended to the nucleic acid barcode construct.
6. The composition of any one of clauses 1 to 3, wherein the nucleic acid nanostructure delivery composition comprises staples that self-assemble to form the nucleic acid nanostructure delivery composition.
7. The composition of clause 6, wherein the staples act as the nucleic acid barcode construct.
8. The composition of any one of clauses 1 to 3, wherein the nucleic acid barcode construct is associated with the nucleic acid nanostructure delivery composition by a high affinity, non-covalent bond interaction between a biotin molecule on the 5′ and/or the 3′ end of the nucleic acid barcode construct and a molecule that binds to biotin on the nucleic acid nanostructure delivery composition.
9. The composition of clause 8, wherein the molecule that binds to biotin is bound to the nucleic acid nanostructure delivery composition by a covalent phosphoramidate bond formed via an EDC-NHS coupling reaction between a terminal phosphate group of a 5′ end of an overhang on the nucleic acid nanostructure delivery composition and an amine group on the molecule that binds to biotin.
10. The composition of clause 8 or 9, wherein the biotin is bound to the nucleic acid barcode construct by a covalent bond.
11. The composition of any one of clauses 1 to 3, wherein the nucleic acid barcode construct is bound to the nucleic acid nanostructure delivery composition by a covalent bond.
12. The composition of clause 11, wherein the covalent bond is formed via an EDC-NHS coupling reaction between a terminal phosphate group of the 5′ end of an overhang on the nucleic acid nanostructure delivery composition and an amine group on an amino terminal nucleotide of the nucleic acid barcode construct.
13. The composition of clause 11, wherein the covalent bond is formed via a click chemistry coupling reaction between an azide group on the nucleic acid nanostructure delivery composition and an alkyne group on the nucleic acid barcode construct.
14. The composition of clause 11, wherein the covalent bond is formed via a click chemistry coupling reaction between an azide group on the nucleic acid barcode construct and an alkyne group on the nucleic acid nanostructure delivery composition.
15. The composition of any one of clauses 1 to 3, wherein the nucleic acid barcode construct is associated with the nucleic acid nanostructure delivery composition by a covalent bond between a carboxy terminated molecule on the nucleic acid nanostructure delivery composition and a primary amine on the nucleic acid barcode construct at the 5′ and/or the 3′ end.
16. The composition of any one of the preceding clauses, wherein the nucleic acid barcode construct comprises two primer binding segments and one or more unique barcode sequences between the two primer binding segments.
17. The composition of clause 16, wherein the primer binding segments range in length from about 15 base pairs to about 30 base pairs.
18. The composition of clause 16 or 17, wherein the primer binding segments are a universal primer binding set.
19. The composition of any one of clauses 16 to 18, wherein the one or more unique barcode sequences comprise unique sequences of about 6 to about 20 nucleotides in length.
20. The composition of any one of clauses 16 to 19, wherein the length of the unique barcode sequences is two times or more greater than the length of the primer binding segments.
21. The composition of any one of clauses 1 to 20, wherein the nucleic acid barcode construct comprises DNA or RNA.
22. The composition of any one of clauses 16 to 21, wherein the unique barcode sequences further comprise a hamming distance of at least 2 to 6 bases between any two unique barcode sequences.
23. The composition of any one of the preceding clauses, wherein the nucleic acid barcode construct further comprises from about 6 to about 12 random bases at the 3′ end of the unique barcode sequences.
24. The composition of clause 23, wherein the about 6 to about 12 random bases at the 3′ end of the unique barcode sequences are for bioinformatic removal of PCR duplicates.
25. The composition of any one of the preceding clauses, wherein the nucleic acid barcode construct ranges in length from about 42 nucleotides to about 210 nucleotides.
26. A method of in vivo screening for a desired nucleic acid nanostructure delivery composition, the method comprising (a) preparing a library comprising two or more types of nucleic acid nanostructure delivery compositions, wherein each nucleic acid nanostructure delivery composition is associated with a nucleic acid barcode construct comprising a different unique barcode sequence, (b) administering the library to an animal, (c) removing cells or tissues from the animal, (d) isolating the nucleic acid barcode constructs from the cells or the tissues of the animal, (e) detecting the nucleic acid barcode constructs in the cells or the tissues of the animal, and (f) identifying the desired nucleic acid nanostructure delivery composition for use as a delivery vehicle.
27. The method of clause 26, wherein the nucleic acid nanostructure delivery composition is associated with the nucleic acid barcode construct according to any one of clauses 4 to 15.
28. The method of clause 26 or 27, wherein the nucleic acid barcode construct is detected by a method selected from the group consisting of the polymerase chain reaction (PCR), isothermal amplification, sequencing, or a combination thereof, to obtain nucleotide sequence data.
29. The method of any one of clauses 26 to 28, wherein the nucleic acid nanostructure delivery composition is loaded with a payload.
30. The method of clause 29, wherein the payload is a luminescent molecule.
31. The method of clause 30, wherein the luminescence is used to track the biodistribution or cell uptake of the nucleic acid nanostructure delivery composition via imaging.
32. The method of any one of clauses 26 to 31, wherein the administration to the animal is via an intramuscular, an intravenous, an intraperitoneal, an oral, or a pulmonary route.
33. The method of any one of clauses 26 to 32, wherein the nucleic acid barcode construct is isolated from the cells and the tissues by mixing with a first organic compound and incubating the organic phase with an aqueous phase of the cell or tissue sample, separating the organic phase from the aqueous phase, mixing the organic phase with a second organic compound, incubating the mixture, precipitating the nucleic acid barcode construct from the mixture, removing the organic phase by evaporation, and resuspending the nucleic acid barcode construct in an aqueous composition.
34. The method of clause 33, wherein the organic phase comprises phenol chloroform.
35. The method of clause 26, wherein the nucleic acid barcode construct is separated from cationic material in the cells or tissues by titrating the aqueous composition of the nucleic acid barcode construct to a pH of greater than 7.4.
36. The method of clause 26, wherein the nucleic acid barcode construct is separated from material in the cells or tissues by binding the nucleic acid barcode construct with a molecule with a binding affinity to the nucleic acid barcode construct greater than the binding affinity to the cell or tissue material.
37. The method of clause 26, wherein the nucleic acid barcode construct is separated from material in the cells or tissues via size exclusion chromatography.
38. The method of clause 26, wherein the nucleic acid barcode construct is separated from material in the cells or tissues via dialysis or diafiltration.
39. The method of clause 26, wherein the nucleic acid barcode construct is separated from material in the cells or tissues via filtration.
40. The method of clause 26, wherein the nucleic acid barcode construct is separated from material in the cells or tissues by digesting proteins using an enzyme.
41. The method of clause 40, wherein the enzyme is Proteinase K.
42. The method of clause 26, wherein the nucleic acid barcode constructs associated with the nucleic acid nanostructure delivery composition are detected by first diluting the isolated nucleic acid barcode constructs by a factor of at least 1000 times, and then amplifying the nucleic acid barcode constructs by PCR using primers.
43. The method of clause 42, wherein the primers from the PCR step are enzymatically digested prior to detection of amplicons.
44 The method of clause 28, wherein the nucleotide sequence data is converted to fast Q files, and the fast Q files are mapped to known unique polynucleotide sequences and the unique polynucleotide sequences are enumerated.
45. The method of any one of clauses 26 to 44, wherein the nucleic acid barcode construct of any one clauses 16 to 25 is used.
46. A composition comprising a non-viral delivery vehicle comprising a nucleic acid nanostructure delivery composition and a payload wherein the nucleic acid nanostructure delivery composition comprises single-stranded or double-stranded DNA or RNA.
47. The composition of clause 46, wherein the nucleic acid nanostructure delivery composition comprises DNA.
48. The composition of clause 46, wherein the nucleic acid nanostructure delivery composition comprises RNA.
49. The composition of clause 46, wherein the nucleic acid nanostructure delivery composition is single-stranded.
50. The composition of clause 46, wherein the nucleic acid nanostructure delivery composition is double-stranded.
51. The composition of any one of clauses 46 to 50, wherein the payload comprises nucleic acids.
52. The composition of clause 51, wherein the nucleic acids comprise DNA or RNA.
53. The composition of clause 51, wherein the payload nucleic acids are used for homology directed repair or as transposable elements.
54 The composition of clause 51, wherein the payload nucleic acids comprise a short guide RNA (sgRNA) and a donor DNA strand.
55. The composition of clause 54, wherein the sgRNA is used for targeting an enzyme to a specific genomic sequence.
56 The composition of any one of clauses 46 to 50, wherein the payload comprises a CRISPR associated enzyme.
57. The composition of clause 55, wherein the targeted enzyme is a CRISPR associated enzyme.
58. The composition of clause 51, wherein the payload comprises a CRISPR associated enzyme, an sgRNA, and a donor DNA strand.
59. The composition of any one of clauses 46 to 50, wherein the payload comprises CRISPR/Cas9.
60 The composition of clause 51, wherein the payloads comprise CRISPR/Cas9, an sgRNA, and a donor DNA strand.
61. The composition of any one of clauses 46 to 50, wherein the payload comprises CRISPR/Cas9 and Cas9 is fused with a deaminase.
62. The composition of clause 51, wherein the payloads comprise a coding sequence for Cas9, an sgRNA, and a donor DNA strand in the form of a plasmid.
63. The composition of clause 51, wherein the payloads consist of one molecule each of CRISPR/Cas9, an sgRNA, and a donor DNA strand.
64. The composition of clause 51, wherein the payloads comprise an antisense oligonucleotide.
65. The composition of clause 51, wherein the payload is of a size selected from the group consisting of 3 kB or more, 3.5 kB or more, 4 kB or more, 4.5 kB or more, 5 kB or more, 5.5 kB or more, 6 kB or more, 6.5 kB or more, 7 kB or more, 7.5 kB or more, 8 kB or more, and 8.5 kB or more.
66. The composition of any one of clauses 46 to 51, wherein the nucleic acid nanostructure delivery composition comprises one or more oligonucleotides with overhangs that bind through complementary base paring with the payload nucleic acids.
67. The composition of any one of clauses 46 to 65, wherein the payload is associated with the nucleic acid nanostructure delivery composition by a high affinity, non-covalent bond interaction between a biotin molecule on the payload and a molecule that binds to biotin on the nucleic acid nanostructure delivery composition.
68. The composition of clause 67, wherein molecule that binds to biotin is bound to the nucleic acid nanostructure delivery composition by a covalent phosphoramidate bond formed via an EDC-NHS coupling reaction between a terminal phosphate group of a 5′ end of an overhang on the nucleic acid nanostructure delivery composition and an amine group on the molecule that binds to biotin.
69. The composition of clause 67 or 68, wherein the biotin is bound to the payload by a covalent bond.
70. The composition of any one of clauses 46 to 65, wherein the payload is bound to the nucleic acid nanostructure delivery composition by a covalent bond.
71. The composition of clause 70, wherein the covalent bond is formed via an EDC-NHS coupling reaction between a terminal phosphate group of the 5′ end of an overhang on the nucleic acid nanostructure delivery composition and an amine group on the payload.
72. The composition of clause 70, wherein the covalent bond is formed via a click chemistry coupling reaction between an azide group on the nucleic acid nanostructure delivery composition and an alkyne group on the payload.
73. The composition of clause 70, wherein the covalent bond is formed via a click chemistry coupling reaction between an azide group on the payload and an alkyne group on the nucleic acid nanostructure delivery composition.
74 The composition of any one of clauses 46 to 65, wherein the payload is associated with the nucleic acid nanostructure delivery composition by a covalent bond between a carboxy terminated molecule on the nucleic acid nanostructure delivery composition and an amine on the payload.
75. The composition of any one of clauses 46 to 74, wherein the nucleic acid nanostructure delivery composition has an aspect ratio of about 2.
76. The composition of any one of clauses 1 to 25 or 46 to 75, wherein the nucleic acid nanostructure delivery composition is coated with one or more polymers.
77. The composition of any one of clauses 1 to 25 or 46 to 76, wherein the nucleic acid nanostructure delivery composition further comprises a targeting component for targeting to cells.
78. A method of treating a patient with a disease, the method comprising administering to the patient the nucleic acid nanostructure delivery composition identified in the in vivo screening method of any one of clauses 26 to 45 or the nucleic acid nanostructure delivery composition of any one of clauses 46 to 77, wherein the nucleic acid nanostructure delivery composition comprises a payload, and treating the disease in the patient.
79. The method of clause 78, further comprising administering a pharmaceutically acceptable carrier to the patient.
80. The method of clause 79, wherein the pharmaceutically acceptable carrier is for parenteral administration or topical administration.
81. The method of clause 78, wherein the patient has a disease or a disorder selected from the group consisting of cancer, a muscular disorder, a pulmonary disorder, a skin disorder, a neurological disease, neurofibromatosis 1, and a hemoglobinopathy.
82 The method of clause 81, wherein the cancer is selected from the group consisting of lung cancer, bone cancer, pancreatic cancer, skin cancer, uterine cancer, ovarian cancer, endometrial cancer, rectal cancer, stomach cancer, colon cancer, breast cancer, cancer of the esophagus, cancer of the endocrine system, prostate cancer, leukemia, lymphoma, mesothelioma, cancer of the bladder, cancer of the kidney, neoplasms of the central nervous system, brain cancer, and adenocarcinoma.
83. The method of clause 81, wherein the skin disorder is a Staphylococcus aureus infection.
84 The method of clause 81, wherein the muscular disorder is muscular dystrophy.
85. The method of clause 78, wherein the nucleic acid nanostructure delivery composition is not cytotoxic to the cells of the patient.

In various embodiments, the nucleic acid nanostructure delivery compositions described herein may comprise any non-viral composition for in vivo delivery of the payloads. By way of example, the nucleic acid nanostructure delivery compositions described herein may be selected from the group comprising synthetic virus-like particles, carbon nanotubes, emulsions, and any nucleic acid nanostructure delivery composition, such as DNA origami structures.

In these embodiments, the nucleic acid nanostructure delivery compositions have a high degree of tunability in structure and function, opportunities to protect payloads from adverse reactions or degradation by the immune system, and cell targeting via surface charge, particle size, or conjugation with various aptamers. These delivery systems also lend themselves to computer aided design, and they have suitable pathways to robust, commercial scale manufacturing processes with higher yields and fewer purification steps than viral manufacturing processes.

A nucleic acid nanostructure delivery composition (e.g., a DNA origami structure), as a delivery platform, is programmable and offers an opportunity for precise scale-up and manufacturing. In this embodiment, the biologic and non-viral nature of the nucleic acid nanostructure delivery composition reduces the chance of adverse immune reactions. In this embodiment, control of each nucleotide that forms a part of the nucleic acid nanostructure delivery composition (e.g., DNA origami nanostructure) allows for the precise design and modification of the structure, including suitable chemical moieties which can make in vivo delivery and endosomal escape possible. In other embodiments, the nucleic acid nanostructure delivery composition can comprise RNA. In various embodiments, the nucleic acid nanostructure delivery composition can be single-stranded or double-stranded, and can comprise DNA or RNA.

In this embodiment, the nucleic acid nanostructure delivery composition can undergo self-base pairing (i.e., a DNA origami structure) to fold into structures that can form the single-stranded or double-stranded scaffold that can encapsulate a payload.

In this embodiment, the nucleic acid nanostructure delivery composition can comprise overhangs that bind through complementary base paring with payload nucleic acids or with the nucleic acid barcode constructs described herein. In this embodiment, the overhangs can be located within a cavity within the nucleic acid nanostructure delivery composition scaffold, and the cavity can be covered by a lid and a hinge allowing the payloads or the nucleic acid barcode constructs to be completely enclosed within the cavity when the lid is shut. In this embodiment, the lid can further comprise oligonucleotide strands that bind through complementary base pairing with other oligonucleotide strands attached to the nucleic acid nanostructure delivery composition scaffold when the lid is in the closed position. DNA nanostructure delivery compositions (e.g., DNA origami structures) are described in U.S. Pat. No. 9,765,341, incorporated herein by reference.

As used herein, the term “complementary base pairing” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

In one aspect, the nucleic acid nanostructure delivery composition of the invention can comprise more than one payload for delivery to target cells, or a nucleic acid payload of 3 kB or more, or another genetic payload, or a small molecule therapeutic for delivery to target cells. In these embodiments, the nucleic acid payload can have a size of 3 kB or more and can be DNA or RNA. In any of the nucleic acid nanostructure delivery composition embodiments described herein, the nucleic acid nanostructure can comprise M13 bacteriophage DNA.

In one illustrative embodiment, the nucleic acid nanostructure delivery composition further comprises a targeting component for targeting to cells. In one aspect, the targeting component can be a nucleotide that is an RNA that forms a ‘stem-and-loop’ structure. In this aspect, the nucleic acid nanostructure delivery composition can be designed so that the polynucleotide strands fold into three-dimensional structures via a series of highly tuned ‘stem-and-loop’ configurations. In this embodiment, the nucleic acid nanostructure delivery composition can have a high affinity for protein receptors expressed on specific cells resulting in targeting of the nucleic acid nanostructure delivery composition and the payload to the specific cells. In this embodiment, the polynucleotide that binds to the target cell receptor can bind in conjunction with a peptide aptamer. In another aspect, the nucleic acid nanostructure delivery composition can be folded so that, in the presence of certain biomarkers such as cell receptors, microRNA, DNA, RNA or an antigen, the self-base pairs are disrupted and the nucleic acid nanostructure delivery composition can unfold, resulting in the triggered release of the payload only in the presence of the specific biomarker. For example, a lock-and-key mechanism for triggered opening of a nucleic acid nanostructure delivery composition (e.g., a DNA origami construct) has been demonstrated previously (Andersen, et al., Nature, Vol. 459, pages 73-76 (2009), incorporated by reference herein). In these embodiments, the use of the nucleic acid nanostructure delivery composition to create three-dimensional structures that target cells and tissues allows for more efficient delivery of payloads with fewer side effects, since the nucleic acid nanostructure delivery composition can have low immunogenicity, and the payload will be released only in the presence of RNA or peptide biomarkers, for example, that exist in the cytosol of target cells and tissues.

In another embodiment, a cell-targeting peptide can be conjugated to a charge neutral peptide nucleic acid, PNA, oligonucleotide instead of a DNA oligonucleotide. PNAs are synthetic polymers of repeating peptide-like amide units (N-(2-aminoethyl)glycine) that mimic nucleic acids in their hybridization affinity and specificity via base-pairing. Their uncharged backbones lead to higher binding affinity with DNA than DNA:DNA so these molecules are suitable for binding to proteins and peptides.

In the embodiment where a nucleic acid nanostructure delivery composition is used, computer aided design tools can predict the nucleotide sequence necessary to produce highly engineered nucleic acid nanostructure delivery compositions. For gene delivery, these nucleic acid nanostructure delivery compositions offer the advantages of encapsulation efficiency, as the size and shape of the structure can be tailored to fit the cargo. In another aspect, loading efficiency can be increased by incorporating payloads into the encapsulating nucleic acid nanostructure delivery composition itself.

In another illustrative embodiment, any of the nucleic acid nanostructure delivery compositions described herein can be coated with one or more polymers to protect the compositions from immune responses or to enhance endosomal escape. In one embodiment, the one or more polymers comprise polyethylene glycol. In another embodiment, the one or more polymers comprise polyethylene glycol poly-L-lysine. In yet another embodiment, the one or more polymers comprise polyethylenimine. In an additional embodiment, the one or more polymers comprise polyethylene glycol poly-L-lysine and polyethylenimine.

In various embodiments, payloads may be combined with the nucleic acid nanostructure delivery compositions using any or all of covalent bonds, electrostatic interactions, and ligand affinity interactions. In one aspect, covalent bonding methods include the use of EDC/NHS to form stable amide bonds between the payload and the nucleic acid nanostructure delivery compositions for improved stability (both “on the shelf” and in vivo), ease of separation and extraction, and sensitive detection. In another illustrative aspect, electrostatic bonding methods include the use of cationic nucleic acid nanostructure delivery compositions that electrostatically complex with the payload. In another embodiment, ligand affinity bonding includes the use of ligands such as avidin and biotin, both covalently bonded to the nucleic acid nanostructure delivery compositions and the payload via EDC/NHS chemistry to yield the stable combination of the payload and the nucleic acid nanostructure delivery compositions. In another embodiment, methods for bonding the payload, including the nucleic acid barcode construct, to the nucleic acid nanostructure delivery composition are provided, including the use of cleavable linkers that can reverse the bond with high specificity, such as the inclusion of nuclease specific oligonucleic acid sequences, allowing the payload, including the nucleic acid barcode construct, to be cleaved and extracted as desired. In another embodiment, cleavable linker and enzyme pairs include amide bonds and amidase enzymes.

In one embodiment, a composition comprising a non-viral delivery vehicle comprising a nucleic acid nanostructure delivery composition, and a nucleic acid barcode construct is provided. In embodiments where the nucleic acid nanostructure delivery composition is barcoded, the nucleic acid barcode construct can be associated with the nucleic acid nanostructure delivery composition via base-pairing. In this embodiment, the base-pairing can occur between a sequence of a single-stranded overhang on the nucleic acid nanostructure delivery composition and a complementary sequence appended to the nucleic acid barcode construct. In other embodiments, the nucleic acid nanostructure delivery composition can comprise staples that self-assemble to form the nucleic acid nanostructure delivery composition, and exemplary stapes are described in Example 1.

In other embodiments, the nucleic acid barcode construct can be associated with the nucleic acid nanostructure delivery composition by a high affinity, non-covalent bond interaction between a biotin molecule on the 5′ and/or the 3′ end of the nucleic acid barcode construct and a molecule that binds to biotin on the nucleic acid nanostructure delivery composition. In this embodiment, the molecule that binds to biotin can be bound to the nucleic acid nanostructure delivery composition by a covalent phosphoramidate bond formed via an EDC-NHS coupling reaction between a terminal phosphate group of a 5′ end of an overhang on the nucleic acid nanostructure delivery composition and an amine group on the molecule that binds to biotin. In this embodiment, the biotin can be bound to the nucleic acid barcode construct by a covalent bond.

In another illustrative embodiment, the nucleic acid barcode construct can be bound to the nucleic acid nanostructure delivery composition by a covalent bond. In this embodiment, the covalent bond can be formed via an EDC-NHS coupling reaction between a terminal phosphate group of the 5′ end of an overhang on the nucleic acid nanostructure delivery composition and an amine group on an amino terminal nucleotide of the nucleic acid barcode construct. In another embodiment, the covalent bond can be formed via a click chemistry coupling reaction between an azide group on the nucleic acid nanostructure delivery composition and an alkyne group on the nucleic acid barcode construct. In yet another embodiment, the covalent bond can be formed via a click chemistry coupling reaction between an azide group on the nucleic acid barcode construct and an alkyne group on the nucleic acid nanostructure delivery composition. In still another embodiment, the nucleic acid barcode construct can be associated with the nucleic acid nanostructure delivery composition by a covalent bond between a carboxy terminated molecule on the nucleic acid nanostructure delivery composition and a primary amine on the nucleic acid barcode construct at the 5′ and/or the 3′ end.

In one aspect, the nucleic acid barcode construct can comprise a polynucleotide barcode and the barcode comprises a unique sequence not present in any known genome for identification of the polynucleotide barcode. In another embodiment, a set of different nucleic acid barcode constructs with different polynucleotide barcodes (e.g., 88 or 96 different polynucleotide barcodes) can be used to allow for multiplexing of samples on one sequencing run.

In various embodiments, the barcodes can be from about 5 to about 100 base pairs in length, from about 5 to about 90 base pairs in length, from about 5 to about 80 base pairs in length, from about 5 to about 70 base pairs in length, from about 5 to about 60 base pairs in length, from about 5 to about 50 base pairs in length, from about 5 to about 40 base pairs in length, from about 5 to about 35 base pairs in length, about 5 to about 34 base pairs in length, about 5 to about 33 base pairs in length, about 5 to about 32 base pairs in length, about 5 to about 31 base pairs in length, about 5 to about 30 base pairs in length, about 5 to about 29 base pairs in length, about 5 to about 28 base pairs in length, about 5 to about 27 base pairs in length, about 5 to about 26 base pairs in length, about 5 to about 25 base pairs in length, about 5 to about 24 base pairs in length, about 5 to about 23 base pairs in length, about 5 to about 22 base pairs in length, about 5 to about 21 base pairs in length, about 5 to about 20 base pairs in length, about 5 to about 19 base pairs in length, about 5 to about 18 base pairs in length, about 5 to about 17 base pairs in length, about 5 to about 16 base pairs in length, about 5 to about 15 base pairs in length, about 5 to 14 base pairs in length, about 5 to 13 base pairs in length, about 5 to 12 base pairs in length, about 5 to 11 base pairs in length, about 5 to 10 base pairs in length, about 5 to 9 base pairs in length, about 5 to 8 base pairs in length, about 6 to 10 base pairs in length, about 7 to 10 base pairs in length, about 8 to 10 base pairs in length, or about 6 to about 20 base pairs in length.

Various embodiments of barcodes are shown below in Table 1 (labeled “Polynucleotide Barcodes”). These barcodes can be used in the nucleic acid barcode constructs alone or in combinations of, for example, two or more barcodes, three or more barcodes, four or more barcodes, etc. In the embodiment where more than one barcode is used, the hamming distance between the barcodes can be about 2 to about 6 nucleotides, or any suitable number of nucleotides can form a hamming distance, or no nucleotides are present between the polynucleotide barcodes.

TABLE 1

| | SEQ | | SEQ | | SEQ |
|---|---|---|---|---|---|
| Polynucleotide | ID | Polynucleotide | ID | Polynucleotide | ID |
| Barcodes | NO: | Barcodes | NO: | Barcodes | NO: |
| GCTACATAAT | 1 | AGCAGTCCCG | 342 | CAAAATAGCG | 683 |
| ATGTTACACA | 2 | TTTGGGCTGT | 343 | GAAGAAGAAG | 684 |
| TGGGGCCCAA | 3 | CTCACGATCT | 344 | CACCCGCACG | 685 |
| TAGTTTATCC | 4 | TGGCGCATAC | 345 | ACGATGCCCG | 686 |
| ACCCCGTCTT | 5 | GCAATTGAAA | 346 | CCTACTACAC | 687 |
| CCGGCCATCA | 6 | TCGGGAGACG | 347 | ATTGAAACAA | 688 |
| GAGCTTGCTC | 7 | CCCGGCGAAA | 348 | GACCGAAGAT | 689 |
| ACGTTCTATA | 8 | TGATGCGGAA | 349 | ACGGCCTGAA | 690 |
| TACAGCAAAA | 9 | AACTGAGGCG | 350 | AGGGGAGGTC | 691 |
| GTTAGGTGGT | 10 | CATATTATTT | 351 | CAATCAACTT | 692 |
| GGAGACCGAC | 11 | AAAAGTCATT | 352 | GGACAACCGA | 693 |
| TGGCCCCTTG | 12 | AAGCGGTGAG | 353 | TCCCTAAGGC | 694 |
| TGGCCGTAAG | 13 | AAGGTAATCA | 354 | GTTCTACACG | 695 |
| CGTTCGTCAA | 14 | CTGACACTTA | 355 | ACTAACCAGT | 696 |
| CGGACGTGGA | 15 | CTGTTTTCTA | 356 | GAAGCTGGAT | 697 |
| AGAGGGGGCA | 16 | CACATGGCAG | 357 | GGAACCATGG | 698 |
| GTTCAGGTCG | 17 | TTCAATCCGG | 358 | CTCTACCTGG | 699 |
| CTCGCAAGAG | 18 | TGTCCGGCAT | 359 | TAATGCCTGC | 700 |
| GCAACGACTT | 19 | TGGTACCGTG | 360 | TAAAGGCAAT | 701 |
| GCCATCCATC | 20 | AAGAGATATT | 361 | CGCCTGGGAA | 702 |
| TTCCGAGCAG | 21 | GATGTACTAC | 362 | TCTTGGGGAA | 703 |
| CTTCTGGACA | 22 | GAAATGGAAT | 363 | AGAGAGAGAG | 704 |
| AACATTAGAC | 23 | TTAAAATACT | 364 | GCGTTGGCGC | 705 |
| AAGCAATAGT | 24 | TGACCGGAAC | 365 | TTACGACAGA | 706 |
| AGGGTAAGAC | 25 | GTCGCCGCAA | 366 | GGAACTCTTA | 707 |
| CGTTGTCTTG | 26 | TAGGATACCG | 367 | GATTGTGGAG | 708 |
| TTTCCCCGCC | 27 | AGTCCAATTG | 368 | GGGCACTGAT | 709 |
| CGAATGGATC | 28 | GGGGGCTATA | 369 | AGACGCACCA | 710 |
| CATCACTTGC | 29 | ACCTTCAGTT | 370 | CCAATTATAA | 711 |
| CTCTCGCACT | 30 | ATGGCAAGTA | 371 | TAGAGACGCA | 712 |
| GTTCACGTGC | 31 | AGAATGTTTT | 372 | CCTCTTGTCG | 713 |
| AATAAGCCTG | 32 | AGTTCGTTTG | 373 | GAGGAAGCTC | 714 |
| GTTAACAATT | 33 | CACTACTGAC | 374 | AGTCCCGAGT | 715 |
| ATTCAGATCC | 34 | GATCAAGAGC | 375 | TGCTTGCAGT | 716 |
| CCTGCTGATT | 35 | ATTTATCGAG | 376 | CCCACTTCCC | 717 |
| CTTGGTCATA | 36 | CCTTTTTCCA | 377 | CGTTGCCGCG | 718 |
| TCTTCCTGTT | 37 | GCACAGAGGT | 378 | CCCCTGGTTC | 719 |
| ACTGCCATGG | 38 | TGATCTGAAT | 379 | ACGACCAATA | 720 |
| CATGTATAGT | 39 | GTTGGAGGGA | 380 | CTTAGGGTTC | 721 |
| GGTAGCGGCA | 40 | TTTTGAAGGT | 381 | AAACATATCA | 722 |
| TCACTCTAAC | 41 | TAAGTCCTAA | 382 | GGGTCGTAGA | 723 |
| AAGGTGCACC | 42 | GGTGTTAGGG | 383 | CTCCGTAGCG | 724 |
| AATGCTCGTT | 43 | TGTATGCACC | 384 | CTGGTCATAA | 725 |
| TGTCTAGAAA | 44 | CCGTGCCATT | 385 | TTGACAGATC | 726 |
| CTGCCTGCCT | 45 | GAAATCACCC | 386 | GAGTAAAGTC | 727 |
| ACTATAAAAG | 46 | TTTGCACGTG | 387 | ATATGGGCTT | 728 |
| TAGTATCGAG | 47 | CGTCTGTTTT | 388 | TACAACTACT | 729 |
| ATCGCAGTCC | 48 | CTACACCACA | 389 | AATTCAGCCG | 730 |
| TCATCAGAAC | 49 | TGCTACAGGG | 390 | GATTGTACTA | 731 |
| TCCTAGACGC | 50 | GGGAATATAT | 391 | TCGTAATGCG | 732 |
| GCCGGGCGGG | 51 | TCATGTATTT | 392 | CGATAACTGC | 733 |
| GCCCAGAAGA | 52 | TCTCCGTTTA | 393 | AACTTGGCGG | 734 |
| CTTAGAGCTG | 53 | TACCTCTCGC | 394 | CGTGGATGTA | 735 |
| GTCTGCGCTT | 54 | GCTTCAACCG | 395 | CCTTCCCGAA | 736 |
| CGCCGTCCTT | 55 | ATGAAGCTAC | 396 | CTAAACCCGT | 737 |
| TTTATCTGCT | 56 | CGGTACAACT | 397 | CAACATTCCC | 738 |
| TGCTTCGGAG | 57 | GTGTGGTCGT | 398 | CTTACCCTCT | 739 |
| GGGGAGAATG | 58 | GGGGTCATGT | 399 | GGAAAGTTCT | 740 |
| GTGGTAAGTG | 59 | AGGCAGCCCA | 400 | CGGATTGGCT | 741 |
| GAAATTAGTA | 60 | CAAGCACGAT | 401 | AATGTAGGGC | 742 |
| GCTATCCTAA | 61 | TCAAATGGAT | 402 | AATGAATCGC | 743 |
| ATCTGTACGA | 62 | GGACTGAATA | 403 | ATCATACACC | 744 |
| AGTTCGGGGC | 63 | CCGTAGACGT | 404 | AGTTGGGCAG | 745 |
| CGAGTCTGTC | 64 | CGGCGTACCG | 405 | AGAAGAAGGG | 746 |
| ATCCTACGCA | 65 | GGCGGCGCCC | 406 | GCGTGCGCTA | 747 |
| ATGGTGGATA | 66 | AGACTTGATC | 407 | CCCCGATAAA | 748 |
| CCTCTAACTA | 67 | ACCTTGCACA | 408 | TACCAAGTGC | 749 |
| ATAGCTGCAC | 68 | TAAGGTGAGT | 409 | TGTGTTTTCG | 750 |
| GACAGAATTT | 69 | TTGTTGTTTC | 410 | CCCAGATGTC | 751 |
| CAATTGGCAT | 70 | GAGGGAATAC | 411 | GCGAGCTTCC | 752 |
| TCTAGTAGAC | 71 | CTCGTACGCG | 412 | GTGTCACGTA | 753 |
| TTATTCATGG | 72 | CCGCGGTTTA | 413 | ATAGGCCGAG | 754 |
| TTGGCAACCG | 73 | TTAAAGTTAA | 414 | GAGCTACCAG | 755 |
| CATAATACAT | 74 | GCATATGGGT | 415 | CGCGGCGGAG | 756 |
| ACAGACTCAC | 75 | AGTCTGAGCC | 416 | TCTTGCACGA | 757 |
| GCGATGCTGC | 76 | TGTCGGTTCG | 417 | TGCCCTAAAG | 758 |
| CATCTTTGCC | 77 | GGTCTCAACC | 418 | TTGCGCTTTG | 759 |
| GTGACTCCAG | 78 | GTAACGGCAT | 419 | CATATAAAGG | 760 |
| GGACGAGTCT | 79 | ACACTGAGAA | 420 | AATAGCGAAT | 761 |
| TAGTGGCGTG | 80 | CCCAACGTCG | 421 | TACGCTAAGG | 762 |
| AACGCAGCTT | 81 | AAGAAACTGC | 422 | ACTTAGTTCG | 763 |
| AGAACAGGTG | 82 | ACCAGCCCAC | 423 | CGTGCGGAAC | 764 |
| AGGCTATGTT | 83 | TGTAGTTACT | 424 | ACCCGATTCG | 765 |
| CCTGGATCTT | 84 | GGCTAGAGGC | 425 | TGCAGAGTTT | 766 |
| CTAGCCGGCC | 85 | GTTCGGCAGA | 426 | GAATCATTAG | 767 |
| ACCAGTTATC | 86 | CCAAAATAGA | 427 | AGTACACTGG | 768 |
| ACGTTATAGC | 87 | CCCATATAAC | 428 | TTGTGCGGTT | 769 |
| TCGAGTTTGA | 88 | GTCACTACCG | 429 | ATGACATGCA | 770 |
| TGAAGCGAGC | 89 | GTAGTGTGGC | 430 | TTCTCGGACG | 771 |
| GACTGGCGAA | 90 | CAATCTCATA | 431 | AGATTGAAGA | 772 |
| GATGGACCTA | 91 | CCATGTTATA | 432 | GGCGGACTGT | 773 |
| GTCCACAACG | 92 | TAAGCAGTGG | 433 | TTTATGGTAA | 774 |
| CCTCCCCAGA | 93 | TCGGCGGCTA | 434 | CAGTAGGGTG | 775 |
| TTATGACGCC | 94 | TATTAAATGC | 435 | GACAGGCAAG | 776 |
| CTTGATCCGT | 95 | GTCGCCATTA | 436 | GATGTGTCGT | 777 |
| AATGCGCAAT | 96 | GGCGTCGTTC | 437 | ACTTGACGGA | 778 |
| GTACCCCTCA | 97 | CTAGTAGATA | 438 | AAGTCCGAAA | 779 |
| CGACAGCTCG | 98 | TCGTCAGTAT | 439 | TGGGTGTAGG | 780 |
| TGACCTGGCT | 99 | GGGGTATCGG | 440 | ACTTACCGCG | 781 |
| TTCATAGCCC | 100 | TGCTCTGCCA | 441 | CTGTGCACCC | 782 |
| CCCAAGAGAA | 101 | TGCCGTAACT | 442 | ATTGCTCTCT | 783 |
| AAACGAAGTA | 102 | CGGTACAGGC | 443 | CAGAAGACAA | 784 |
| GACGTTTACA | 103 | TCCTAATTTG | 444 | TTACGCTATA | 785 |
| GATCGATTTG | 104 | TCTTTCTGGA | 445 | ACGTGGAAAT | 786 |
| CACTGTCACC | 105 | CCGCGACTTG | 446 | TGAGGCTGGT | 787 |
| TGTGAGAGTT | 106 | ACCTATAGCG | 447 | ATTATGAGAT | 788 |
| GACGTAACCT | 107 | GCCGGCACCT | 448 | GACTTGTAGT | 789 |
| CAGACTCTGC | 108 | TTTGATAGGC | 449 | TCGCTGAGGA | 790 |
| TATGCCAATA | 109 | ACTGTGAGCT | 450 | CCCAACTCTA | 791 |
| ACAGGTGATG | 110 | TTATCGTTCA | 451 | GATAGGGAGG | 792 |
| GTCATCGCGT | 111 | ACTAGTGGCC | 452 | TAGAAATCAG | 793 |
| TCTTATAAAC | 112 | CCTCCGTGGT | 453 | GTCGCTAGAA | 794 |
| GTGTAGACTG | 113 | TTAGGGTATG | 454 | AAAATAGAAA | 795 |
| AAACAACCGG | 114 | GAATCAGGCG | 455 | GCTCCTGGGT | 796 |
| ATCCTGTACC | 115 | GGCTGACCAA | 456 | CGCGCTCGCG | 797 |
| TTATAAGAAT | 116 | TGCCAGACCG | 457 | GGCAAACGCA | 798 |
| ATAAGTAGGC | 117 | TCCCTACGCG | 458 | TTTACTACCT | 799 |
| TCTCGTAAGG | 118 | TCCGCTGGAG | 459 | ATCCTAAACT | 800 |
| GATCCGCCGC | 119 | GGATCAAAAC | 460 | CTCCGTATGT | 801 |
| TGTCAGGTTT | 120 | TTCACCTCAC | 461 | TATCGTCCAG | 802 |
| TCCGAAGCCC | 121 | GACACACGGC | 462 | GCCGGCGGTA | 803 |
| TCCATGTCCA | 122 | TGGGCGATTA | 463 | TGCTCCATTT | 804 |
| GTGATGGTAC | 123 | TAAGATCTTC | 464 | TGGCTGTTGT | 805 |
| CTCCACATAC | 124 | CTCCGACTAC | 465 | TACTGCGCAA | 806 |
| TTCGGATGAG | 125 | GGGCCATCAT | 466 | TATACGGCTT | 807 |
| ACGACATCGC | 126 | TCAGGCCAGA | 467 | GGTTATTACC | 808 |
| GAGATGCACA | 127 | CTTGTGGGGC | 468 | ATCAGGAGGA | 809 |
| TTTGTATGGC | 128 | AGATAGTCTG | 469 | CTATTGCCAG | 810 |
| CTTTTCTAGA | 129 | GCGTCAAAGT | 470 | ACGTACACAC | 811 |
| AGTCTAATCA | 130 | ACGAAAATTT | 471 | CAGCCTAGCT | 812 |
| GACTTAGCCA | 131 | GAGTCTGGTG | 472 | GAAAAACAAC | 813 |
| TATCACAGTA | 132 | ATCGAGCGAC | 473 | CGTTCAGTTA | 814 |
| AAGCTCGAGT | 133 | GGTCCTCAGA | 474 | CAATCAGAAT | 815 |
| TGTTACGACA | 134 | TGATTTTGTC | 475 | GGGCTACTCT | 816 |
| AAGGATAGTC | 135 | GCATTTCTCA | 476 | CCCCATTGGG | 817 |
| GCACTTAGCC | 136 | GCATGCCAGT | 477 | TAGGGAACGG | 818 |
| GAGGGATCCG | 137 | ATTAGACGAC | 478 | CAGCTGATAC | 819 |
| ATTCTAGAAG | 138 | AAAGCCCATA | 479 | ATTCCTGTGA | 820 |
| GATAACTGAT | 139 | CACTACATTC | 480 | TCAGAGCCGT | 821 |
| ATCTGACTGT | 140 | CACGGTTTCT | 481 | CATGAAAAGC | 822 |
| CAAAGCGAAC | 141 | CCCACCAGTG | 482 | TGACCTGTGA | 823 |
| GAAATTGCGA | 142 | CTCACTTGTC | 483 | GCATTAGCAG | 824 |
| GGGTCCAGTC | 143 | GATAGACTCT | 484 | GACAGAACCA | 825 |
| ATCAGGTAGC | 144 | ATTTCCATTT | 485 | TCCAGTATAT | 826 |
| GAAAGGTCCT | 145 | ATATGTGGCC | 486 | TGTTCCGCTA | 827 |
| GGCTACCACA | 146 | CGGGACGAAC | 487 | GATATCCATT | 828 |
| TTATTGCTGA | 147 | AGAACCGTGA | 488 | CATATGGACC | 829 |
| CGCCGCGTTT | 148 | TAGTGTACTG | 489 | GATATAGTAA | 830 |
| TTTTCAAAAG | 149 | AACTAATCGA | 490 | CACCTTTTTT | 831 |
| CTGGGCTAAA | 150 | CGAAGTGACG | 491 | AGCTTGCGGG | 832 |
| CCCGATGAGA | 151 | CGGAGCCTCG | 492 | CGCACAGGGA | 833 |
| TGGGAAATAT | 152 | ATCACACGAG | 493 | TCTGGGTGCT | 834 |
| GTACGAGCGG | 153 | CGACGAGTTC | 494 | TGAGTCGTTT | 835 |
| GCGTGCAGCT | 154 | GCTTCCCGTG | 495 | TTACAATGTG | 836 |
| AGTCTGCGGA | 155 | GATTCATACC | 496 | CTTGCAAACA | 837 |
| TAACTATTTA | 156 | GAGAGAAGCG | 497 | TGTCGAGCTG | 838 |
| GAGTTGCCGG | 157 | GAAGTGGCCT | 498 | ACTTTAACCT | 839 |
| CAGCCCGGCG | 158 | GGACGACGCC | 499 | ATATAAGTGC | 840 |
| TCACCTACAT | 159 | TAGGGTCTCA | 500 | GGAAGGGCGT | 841 |
| AGTGGCTAAC | 160 | AACTACAGGT | 501 | TTTGACTTGA | 842 |
| AGAATGTGAG | 161 | GTGGCCTGTG | 502 | GTATAAACGG | 843 |
| TAGTTTCGCA | 162 | CTTTACCAGC | 503 | TAACCGGATG | 844 |
| CTTCATTTCT | 163 | CGCGTTACTG | 504 | TTCTCATCAG | 845 |
| GCCATGATAT | 164 | TTGCTCCCGT | 505 | CTCGGTTACG | 846 |
| ACGGCAAATC | 165 | CATCAAACAA | 506 | ATATGGTTCT | 847 |
| ATCGATAGTA | 166 | GCTTTATGAT | 507 | CGCCCCCGAA | 848 |
| CCTAAAGGCA | 167 | CTGCATACTG | 508 | ACCTCGATCG | 849 |
| TACGAGCGGT | 168 | GGTGGCTCAG | 509 | CTCGAATAAT | 850 |
| TTTGTCGTCG | 169 | GGACGATCAA | 510 | GCCCGAGCTT | 851 |
| TACAAGCTTG | 170 | CCGACTGGTG | 511 | AACAGTCAAC | 852 |
| GACCAACACG | 171 | GGAACAACCG | 512 | CTGGAACCTC | 853 |
| GAACGACGAA | 172 | GAACGAGACC | 513 | AATAACGGGG | 854 |
| TCGGAACGCA | 173 | CACCAAGAAA | 514 | ACGCCCCACT | 855 |
| ATCCGGTGGT | 174 | ATGCATTACC | 515 | GGCAACATGA | 856 |
| TAAAACGTAG | 175 | GTATCATGCC | 516 | GCTATTTCGC | 857 |
| TATGTGAGCC | 176 | AGTAGATGTT | 517 | TTCCACTTTA | 858 |
| GAGGCATCGA | 177 | CTCTAGATGT | 518 | GCCGATGGAT | 859 |
| GAATGGGTGG | 178 | GCTACTTGTG | 519 | AAGTTGGTAA | 860 |
| AACGACACAA | 179 | TATGAAACGT | 520 | CACTAGCTAG | 861 |
| GTACGATGCA | 180 | CCTCGTTGAT | 521 | ACATGCCCCT | 862 |
| AGAAGGCGCC | 181 | CTAGAGCCAT | 522 | TTCATTACTC | 863 |
| CCGCAATGGA | 182 | TAGAGTTATA | 523 | GGTTTAATAT | 864 |
| TACGGATTTT | 183 | AACGAGAGGC | 524 | CCTGCAGTGA | 865 |
| GTCGTTAGCT | 184 | GGTCTACCGT | 525 | TCTTTAAGTT | 866 |
| GGACTAGGGC | 185 | GCCCCCTCAC | 526 | TGGCGATCGA | 867 |
| ATTGGTATTC | 186 | CATAGGAATT | 527 | CTTTTTAGCT | 868 |
| ATCCCAGAGA | 187 | TCCGGCTCGT | 528 | CCCAGTCTCT | 869 |
| GTCCCAGCTC | 188 | TGAGAGTCGG | 529 | AAATGTTTCG | 870 |
| CACGAGGAAT | 189 | CGTAGAAATA | 530 | ATATAAGACG | 871 |
| TACAATTGCA | 190 | CTTTACATGA | 531 | TCACTTTACA | 872 |
| ATTCCTGAAT | 191 | GAGCGCCGTC | 532 | CCTGGCGCCC | 873 |
| TAGCGAGGCG | 192 | GGCTCTCGGC | 533 | GGATTACTGG | 874 |
| CTGGATGGGC | 193 | AGAGCTTGTT | 534 | GAATGATCTT | 875 |
| GCGACGGCCA | 194 | AATCAGCCAC | 535 | GCTCGGATCG | 876 |
| ACCTGCACAA | 195 | AGAAGAGCCA | 536 | CAGCTGCGAG | 877 |
| CATGACAGAC | 196 | TCGTATGAGT | 537 | ACCCTTACTA | 878 |
| TTACCAACGT | 197 | TTCTTCCTCG | 538 | AGGTGAAACT | 879 |
| CAGGTGTGTG | 198 | ACACAAAAGC | 539 | CGAATTTGAT | 880 |
| CGAGGGACGG | 199 | CGCGGGACCC | 540 | CGCTGTGCGG | 881 |
| CGTCTCGGTA | 200 | GTCGCGACAC | 541 | TTACCGCACC | 882 |
| TAAGCTATCT | 201 | CCGGAGGAAA | 542 | GGAATCTTAA | 883 |
| TACTCCCCTA | 202 | CGGCGTATGA | 543 | CTCAACACCC | 884 |
| TTATATTCAT | 203 | TAGGCATTCT | 544 | CGTGCCCTTG | 885 |
| AGCGATCTGC | 204 | AAAGGAGGGA | 545 | GCAGGCTCGA | 886 |
| TCTTCTGATC | 205 | ACCTTTACGG | 546 | ACCAACGAAG | 887 |
| ATAGTTCCCA | 206 | CTACCGTTAA | 547 | CCTGTAATTT | 888 |
| TTTACGGGTG | 207 | GAGCTTCGCC | 548 | GGGTGGGATG | 889 |
| GTGTCCCCTG | 208 | GCCATAGAAG | 549 | TTGCTCACCG | 890 |
| GCGGGGGTCG | 209 | TTTAGCGTAT | 550 | TTACGACCAC | 891 |
| CATTGATCTA | 210 | GCAAACAGAT | 551 | TTTTCTAACC | 892 |
| AGGGACGGTG | 211 | TAGGTCATGG | 552 | GCTTTAGATA | 893 |
| CAGTTACTTT | 212 | CTCTAACAGA | 553 | CACGTATTGG | 894 |
| CCATACTTCC | 213 | GGCTCATGAA | 554 | AAATATCTCC | 895 |
| ATCAGAATTA | 214 | CAATGTCTCA | 555 | GCTGGAAAAC | 896 |
| AAACTAGGCA | 215 | TGATCGTATT | 556 | GAGCGCATTA | 897 |
| AATGTCGTTG | 216 | GCGCTTTTCA | 557 | GTGGAGGGGT | 898 |
| CACATGGGTC | 217 | AAGATTATAT | 558 | TCCACTGGGA | 899 |
| GGTCGCTGGT | 218 | ACTAGCTGAC | 559 | CAATAGCGGA | 900 |
| ACTGTATTAC | 219 | GGTGAGCTCA | 560 | CATCTAGTTT | 901 |
| CCGAGACGCG | 220 | CGCTTTCGCT | 561 | GAAGTTCCGG | 902 |
| ACTCCAACCC | 221 | TGATTCAAAA | 562 | AGCGAGATTC | 903 |
| ATATTACAAG | 222 | ACTGAACAGG | 563 | TTAAGGTCGG | 904 |
| CCATGGATAG | 223 | ATTCGAGCTA | 564 | AATGGTTAGG | 905 |
| CCGTCTCAAT | 224 | TGTAGGCTAA | 565 | CGTTATTATA | 906 |
| GATCGTCGGG | 225 | ACAAAGCTTT | 566 | ACGGAAAGGA | 907 |
| TCTTGTTTTG | 226 | GCCCGAGGGA | 567 | CCTTGTCCCG | 908 |
| AATATTGCTC | 227 | GCCCGCTGGG | 568 | ATACTTTTTT | 909 |
| AACGTCGTCT | 228 | ACCCCGCTGA | 569 | CTGGGTCTGG | 910 |
| AATATTTTTG | 229 | CTTATGCCCT | 570 | AACCATTGCG | 911 |
| CGTAACGTGC | 230 | CCGCCATAGC | 571 | AGACCGGGCC | 912 |
| GCGTGGTTAT | 231 | CTTAATGATT | 572 | TGGGACACAC | 913 |
| CAAAACATTA | 232 | CAGTCCACAA | 573 | TGCGCAGTTG | 914 |
| CGTATCCTGA | 233 | ATGGACGGAC | 574 | CGTTCGCCTT | 915 |
| TCGCTTACAA | 234 | CGGCCTCTCG | 575 | TCTCACTCGT | 916 |
| TCCATTGTGT | 235 | TAGTCGCCAT | 576 | ACACCGACGT | 917 |
| GCCCCCATTC | 236 | GTTGATCTTC | 577 | TTCAGCCCCT | 918 |
| TGACGTCTAT | 237 | ACTTGCCAAG | 578 | AGGCGACTAA | 919 |
| TGGGCCGAGG | 238 | ATGACTGGTT | 579 | TGCTATCAAG | 920 |
| AAGTGTCAAG | 239 | TGTCGTAGGA | 580 | GTCCAGTAGC | 921 |
| GACAGTAGAG | 240 | AGCAAACACG | 581 | CGTGTGGGCG | 922 |
| CGCAGCCATC | 241 | TACTGATGAA | 582 | GTGGTTCTCC | 923 |
| GAGGCAGAAC | 242 | GTATCCCATA | 583 | GCAGCCGACG | 924 |
| GTTGAAATTG | 243 | TAGCCAGGTT | 584 | GCTGTCCACG | 925 |
| ATCTGATAAA | 244 | CGTGTGGCGA | 585 | CGACACTCAT | 926 |
| AGCTGTCTCT | 245 | ATCGAATTGC | 586 | CATGGCACCT | 927 |
| TTTTAGGTTA | 246 | CCCCAATATT | 587 | TGTGACGTGT | 928 |
| TATCTGTCCG | 247 | CCCGTTTCTC | 588 | TTTGGACTAA | 929 |
| AAAACATATG | 248 | TCCGCATCTA | 589 | TTCATGCCCG | 930 |
| GTAAAGAAGA | 249 | CAAGCCTCAT | 590 | TTGATCGTGG | 931 |
| TCGACGTGCA | 250 | TTTCAATCCC | 591 | TAGCATAGGA | 932 |
| TAGATCTTAA | 251 | CCTTCCCATC | 592 | GTAGTTGCAA | 933 |
| CACTGGTCAC | 252 | AGGTACAAGA | 593 | GGGACAGCTA | 934 |
| ATTCTGATGT | 253 | GTGTAATGGA | 594 | AAACCCCCAA | 935 |
| ATGGCCCTGA | 254 | AAACTGAGCT | 595 | ACTCTCACAA | 936 |
| GGTGATGAGA | 255 | ATCTCTGCCC | 596 | ATCATTGCCA | 937 |
| CACCGTGGGG | 256 | CGACATTTGC | 597 | CCAGTTTGCG | 938 |
| GCTTGCTCGG | 257 | TGTGAACCCG | 598 | ACATTAGTCA | 939 |
| CCAGTTGAAC | 258 | TGACACCCCA | 599 | CTCCAGGGTA | 940 |
| CGTCTGTACC | 259 | TAGGCCAAAG | 600 | GAAGGGCCAA | 941 |
| CCAACGCGGC | 260 | GAAATTGTAG | 601 | CAGTCTCCCC | 942 |
| ACGTGATCGA | 261 | GCGTCTGATT | 602 | GAGACATTCC | 943 |
| CCATCGAATC | 262 | TCTCATTGTT | 603 | AACGGTGTTG | 944 |
| CGGTGTCTGC | 263 | CTGACATCTC | 604 | AGCATTATCA | 945 |
| AAACCACCTC | 264 | GTATCCAGTG | 605 | CTATACCGAG | 946 |
| TCAATGTTCC | 265 | GATGGCCGTT | 606 | AACTGGATCA | 947 |
| TTCGACATGT | 266 | TCACCCTCTC | 607 | GTCTTGTCGG | 948 |
| AGGCACGATA | 267 | GGCACTATTC | 608 | GACGAGCCGC | 949 |
| CACGAGATCA | 268 | AAATAACTGT | 609 | GGAACACTGT | 950 |
| CATGCTGGGG | 269 | CAGCTCCATT | 610 | TAAATGCGTT | 951 |
| TACCATGGTT | 270 | CTCTTGACTC | 611 | GCGAACACAG | 952 |
| TTGCCCATAT | 271 | TTTCCTATAC | 612 | TTCTCTCAAC | 953 |
| TGCACATTCG | 272 | CCATACCCGA | 613 | GTCGTACTGA | 954 |
| GTTATGTTGG | 273 | TCGCCGAGCG | 614 | TGTGGCGTAA | 955 |
| TGAGTTATGA | 274 | CGCTGAAGCC | 615 | TGAGCGGCGT | 956 |
| GATGGCCCCC | 275 | TCTGGCCCCA | 616 | CCTCGTGAAC | 957 |
| GATGGGTTAC | 276 | GCTACATTGA | 617 | GAGCAATGAA | 958 |
| AGCTACGTTG | 277 | CGCATCATAA | 618 | CGAGACCTAA | 959 |
| ACCCCATGCA | 278 | GCAAAGGGCC | 619 | AACTGAGCGC | 960 |
| TACTACCGTT | 279 | AACGGCGCAG | 620 | TAAAGCTCGT | 961 |
| TCGCTTCTAC | 280 | CGACTGACAT | 621 | CTCTTTACGT | 962 |
| CTGGCAGTGC | 281 | ATGACAGGGC | 622 | CCCCGTGGAA | 963 |
| TCTATATATA | 282 | CAAGTTCTCC | 623 | TCGGTTCGTC | 964 |
| GGATTAGTTC | 283 | TCGCCGCTTT | 624 | CTGCTTACAC | 965 |
| GTGTTACGCT | 284 | ATGCCGGAAA | 625 | ACACCGTAAT | 966 |
| TCGACTCCGT | 285 | GCGGTTACTA | 626 | CCTGGTCGGC | 967 |
| GGTAGCAGGC | 286 | GACATTACAA | 627 | GGTTATTTGG | 968 |
| TATTGGATTC | 287 | CAGAGAGGGC | 628 | GCAACTGAGT | 969 |
| GTTCGATCGA | 288 | GCACCGCCTC | 629 | ATAAGGCCTC | 970 |
| ATATTAATAT | 289 | CGGTCCGAGC | 630 | CGTGCGAAGG | 971 |
| AGAACGATTG | 290 | TGTCCGGTGC | 631 | GTCACACACT | 972 |
| GTAAAGTGTA | 291 | GGTCGGTTGC | 632 | CATACGGCAA | 973 |
| CCCATGTGCC | 292 | GCTCAGCTAA | 633 | GAACTGCCCA | 974 |
| GTGGCCTCGC | 293 | AGCAGTTCGT | 634 | AATATGTGAA | 975 |
| GACACTAGGA | 294 | AAATCGATGA | 635 | CCGATCCTGT | 976 |
| ATATTCTGAC | 295 | GCTCGGTATG | 636 | CAAAGAGCCT | 977 |
| TAAGTAGACG | 296 | CCCGCCGCGG | 637 | TAACTTAGAG | 978 |
| TAACGGTCTA | 297 | GTGTGATAGG | 638 | CAGCATGTAG | 979 |
| TAGTTTCATT | 298 | TTGGACTCCA | 639 | CCCCATGCAG | 980 |
| TTGGATCCGA | 299 | TGCTTATCTA | 640 | TCTGAACCAC | 981 |
| CGTGACAACC | 300 | CAAAAGGCGT | 641 | GCGTGCAAAA | 982 |
| CGCGCTCAGA | 301 | TAGGGGGCCT | 642 | GCTAGTACCG | 983 |
| CGTTCTTAAT | 302 | AAGTATTAAT | 643 | TTTCCCGCGC | 984 |
| ACAAGAGTTT | 303 | GTTTAGCCCG | 644 | CCTTAGTAGG | 985 |
| AGGGTTATAG | 304 | CGCTAATATG | 645 | TTGTGTCTTG | 986 |
| ACCACGACTC | 305 | ACAACACGTT | 646 | GCAACGAAGC | 987 |
| GTACTCGGGG | 306 | AGAGATGCTC | 647 | TGAAACCCTT | 988 |
| ACAAATATCT | 307 | TGCCTGATAT | 648 | TTCTACGATC | 989 |
| GATCGGGGTG | 308 | CTTGTAAGTA | 649 | ATTAAAGGTG | 990 |
| ATGTAACTCC | 309 | CATATTGCCG | 650 | TATCTAACGG | 991 |
| ATGAAGAAGC | 310 | CTTAGAAAGT | 651 | AGTGCTCCTG | 992 |
| ATGTATTGTC | 311 | ATGTTGTATT | 652 | CCGTCCCTCT | 993 |
| TGCATTGGAA | 312 | CGCATTGAAG | 653 | CTAACGAGCG | 994 |
| GCGGACGATC | 313 | TTATGTTGGT | 654 | AAGTCCGGCT | 995 |
| CCGTACTTGA | 314 | TCGCCTCAGA | 655 | GGCGTATAAG | 996 |
| TTTGCCCCCG | 315 | TTCGTTGAGG | 656 | AGATATTAGG | 997 |
| ACCTCACGCG | 316 | GGTGCCGGGC | 657 | TCCTAACAGC | 998 |
| ATTAAGGGGC | 317 | ACCATTGTAA | 658 | GAGGATACGC | 999 |
| CGTGGACATG | 318 | TTGATTGTCA | 659 | CGCTCTTTAA | 1000 |
| TTAGCCCTTC | 319 | CGGCTCACCT | 660 | ACCGGCAGGC | 328 |
| CGAGAGTTTG | 320 | CTATCACATG | 661 | GCTAAAATCT | 329 |
| TGCATCCTCT | 321 | GTAGACAGAA | 662 | GCCGTTGACG | 330 |
| TGCGATTCCG | 322 | CCTTTACCAA | 663 | GGAGTTGTTG | 331 |
| TTATTACGTT | 323 | GCACATCGAC | 664 | TACTTGAGAA | 332 |
| TGATGTGGTT | 324 | TCTCACTTTC | 665 | CGGGTGCGCT | 333 |
| GGGCGTCAAT | 325 | TTCGAGTACT | 666 | AAAAGCGTCT | 334 |
| CCCTTGAAAT | 326 | TAGAAGAGCA | 667 | GTAAAGATAG | 335 |
| TCTTTGGGGC | 327 | AACCCCACCA | 668 | GCCTGGTCAG | 336 |
| ACCGGCAGGC | 328 | CTGTATCAGT | 669 | GGCAAAAAGG | 337 |
| GCTAAAATCT | 329 | ACATAATGAG | 670 | ACCCTTCTCT | 338 |
| GCCGTTGACG | 330 | AGCCTTCCGC | 671 | TCACATAGTG | 339 |
| GGAGTTGTTG | 331 | CAGTGCTTTT | 672 | TCGTCTGTGC | 340 |
| TACTTGAGAA | 332 | TAGTCCGTGT | 673 | TGCTCGGATC | 341 |
| CGGGTGCGCT | 333 | CGGAATCGGT | 674 | GGCGTATAAG | 996 |
| AAAAGCGTCT | 334 | CTTGCGGAGA | 675 | AGATATTAGG | 997 |
| GTAAAGATAG | 335 | AAAAATTTGG | 676 | TCCTAACAGC | 998 |
| GCCTGGTCAG | 336 | TGTTTTCCGC | 677 | GAGGATACGC | 999 |
| GGCAAAAAGG | 337 | ATGCTAGGCG | 678 | CGCTCTTTAA | 1000 |
| ACCCTTCTCT | 338 | GACTAATTTC | 679 | GGCGTATAAG | 996 |
| TCACATAGTG | 339 | CTGTAGTAAC | 680 | AGATATTAGG | 997 |
| TCGTCTGTGC | 340 | CGGATGACTT | 681 | TCCTAACAGC | 998 |
| TGCTCGGATC | 341 | TCAGAGTGGA | 682 | GAGGATACGC | 999 |

In another embodiment, a random sequence fragment can be linked to the 5′ and/or the 3′ end of the barcode and the random sequence fragment can, for example, be used for bioinformatic removal of PCR duplicates. The random sequence fragment can also be used to add length to the nucleic acid construct and can serve as a marker for bioinformatic analysis to identify the beginning or the end of the barcode after sequencing. In another embodiment, the nucleic acid barcode construct comprises at least a first and a second random sequence fragment, and the first random sequence fragment can be linked to the 5′ end of the barcode and the second random sequence fragment can be linked to the 3′ end of the barcode. In another embodiment, one or at least one random sequence fragment is linked to the 5′ and/or the 3′ end of the barcode. In one aspect, the random sequence fragments can be extended as needed to make the nucleic acid barcode construct longer for different applications such as whole genome sequencing where short inserts may be lost.

In various embodiments, the random sequence fragments can be from about 5 to about 20 base pairs in length, about 5 to about 19 base pairs in length, about 5 to about 18 base pairs in length, about 5 to about 17 base pairs in length, about 5 to about 16 base pairs in length, about 5 to about 15 base pairs in length, about 5 to about 14 base pairs in length, about 5 to about 13 base pairs in length, about 5 to about 12 base pairs in length, about 5 to about 11 base pairs in length, about 5 to about 10 base pairs in length, about 5 to about 9 base pairs in length, about 5 to about 8 base pairs in length, about 6 to about 10 base pairs in length, about 7 to about 10 base pairs in length, or about 8 to about 10 base pairs in length.

In another illustrative aspect, the barcode may be flanked by primer binding segments (i.e., directly or indirectly linked to the barcode) so that the nucleic acid barcode construct comprising the barcode can be amplified during a polymerase chain reaction (PCR) and/or sequencing protocol. In one aspect, the primer binding segments can be useful for binding to one or more universal primers or a universal primer set. In one illustrative embodiment, the universal primers can contain overhang sequences that enable attachment of index adapters for sequencing. In one embodiment, the adapters can be NGS adapters (e.g. Illumina) positioned internally but towards the end of either the 5′ or the 3′ primer, not as the terminating structure, to avoid the formation of primer dimers. In this aspect, the primers can be any primers of interest. In this embodiment, the first primer binding segment can be linked at its 3′ end to the 5′ end of a first random sequence fragment and the second primer binding segment can be linked at its 5′ end to the 3′ end of a second random sequence fragment with the barcode between the random sequence fragments. In another embodiment, the first primer binding segment can be linked at its 3′ end to the 5′ end of the barcode and the second primer binding segment can be linked at its 5′ end to the 3′ end of a random sequence fragment linked to the 3′ end of the barcode. In another embodiment, the first primer binding segment can be linked at its 3′ end to the 5′ end of a random sequence fragment and the second primer binding segment can be linked at its 5′ end to the 3′ end of the barcode where the barcode is linked at its 5′ end to the 3′ end of the random sequence fragment. In yet another embodiment, the first primer binding segment can be linked at its 3′ end to the 5′ end of the barcode and the second primer binding segment can be linked at its 5′ end to the 3′ end of the barcode.

In embodiments where primer binding segments are included in the nucleic acid barcode construct, the primer binding segments can range in length from about 15 base pairs to about 30, from about 15 base pairs to about 29 base pairs, from about 15 base pairs to about 28 base pairs, from about 15 base pairs to about 26 base pairs, from about 15 base pairs to about 24 base pairs, from about 15 base pairs to about 22 base pairs, from about 15 base pairs to about 20 base pairs, 16 base pairs to about 28 base pairs, from about 16 base pairs to about 26 base pairs, from about 16 base pairs to about 24 base pairs, from about 16 base pairs to about 22 base pairs, from about 16 base pairs to about 20 base pairs, 17 base pairs to about 28 base pairs, from about 17 base pairs to about 26 base pairs, from about 17 base pairs to about 24 base pairs, from about 17 base pairs to about 22 base pairs, from about 17 base pairs to about 20 base pairs, 18 base pairs to about 28 base pairs, from about 18 base pairs to about 26 base pairs, from about 18 base pairs to about 24 base pairs, from about 18 base pairs to about 22 base pairs, or from about 18 base pairs to about 20 base pairs.

An exemplary sequence of a nucleic acid barcode construct is shown below. The /5AmMC6/ is a 5′ amine modification for attachment to the nucleic acid nanostructure delivery composition. The *'s are phosphorothioate bond modifications for stability. The A*G*A*CGTGTGCTCTTCCGATCT sequence (SEQ ID NO: 1001) is the 5′ primer binding segment sequence. The GCTACATAAT (SEQ ID NO: 1) is an exemplary barcode sequence. The N's represent the random sequence fragment. The AGATCGGAAGAGCGTCG*T*G*T (SEQ ID NO: 1002) is the 3′ primer binding segment sequence.

(SEQ ID NO: 1003)
/5AmMC6/A*G*A*CGTGTGCTCTTCCGATCTGCTACATAATNNNNNNNN
NNAGATCGGAAGAGCGTCG*T*G*T

In all of the various embodiments described above, the entire nucleic acid barcode construct can range in length from about 30 base pairs to about 350 base pairs, from about 30 base pairs to about 300 base pairs, from about 30 base pairs to about 270 base pairs, about 30 base pairs to about 240 base pairs, about 30 base pairs to about 230 base pairs, about 30 base pairs to about 220 base pairs, about 30 base pairs to about 210 base pairs, about 30 base pairs to about 200 base pairs, about 30 base pairs to about 190 base pairs, about 30 base pairs to about 180 base pairs, about 30 base pairs to about 170 base pairs, about 30 base pairs to about 160 base pairs, about 30 base pairs to about 150 base pairs, about 30 base pairs to about 140 base pairs, about 30 base pairs to about 130 base pairs, about 30 base pairs to about 120 base pairs, from about 30 base pairs to about 110 base pairs, from about 30 base pairs to about 100 base pairs, from about 30 base pairs to about 90 base pairs, from about 30 base pairs to about 80 base pairs, from about 30 base pairs to about 70 base pairs, from about 30 base pairs to about 60 base pairs, from about 30 base pairs to about 50 base pairs, from about 30 base pairs to about 40 base pairs, 40 base pairs to about 120 base pairs, from about 40 base pairs to about 110 base pairs, from about 40 base pairs to about 100 base pairs, from about 40 base pairs to about 90 base pairs, from about 40 base pairs to about 80 base pairs, from about 40 base pairs to about 70 base pairs, from about 40 base pairs to about 60 base pairs, from about 40 base pairs to about 50 base pairs, 50 base pairs to about 120 base pairs, from about 50 base pairs to about 110 base pairs, from about 50 base pairs to about 100 base pairs, from about 50 base pairs to about 90 base pairs, from about 50 base pairs to about 80 base pairs, from about 50 base pairs to about 70 base pairs, from about 50 base pairs to about 60 base pairs, or about 42 base pairs to about 210 base pairs.

In another embodiment, a method of in vivo screening for a desired nucleic acid nanostructure delivery composition is provided. The method comprises (a) preparing a library comprising two or more types of nucleic acid nanostructure delivery compositions, wherein each nucleic acid nanostructure delivery composition is associated with a nucleic acid barcode construct comprising a different unique barcode sequence, (b) administering the library to an animal, (c) removing cells or tissues from the animal, (d) isolating the nucleic acid barcode constructs from the cells or the tissues of the animal, (e) detecting the nucleic acid barcode constructs in the cells or the tissues of the animal, and (f) identifying the desired nucleic acid nanostructure delivery composition for use as a delivery vehicle. In this embodiment, any of the nucleic acid nanostructure delivery compositions can be used and any of the nucleic acid barcode constructs described herein can be used.

In various embodiments, any suitable route for administration of the library of nucleic acid nanostructure delivery compositions associated with nucleic acid barcode constructs for the method of in vivo screening for the nucleic acid nanostructure delivery compositions associated with a nucleic acid barcode construct, or for the method of treatment described below can be used including parenteral administration. Suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques. In other embodiments, oral or pulmonary routes of administration can be used.

In one aspect, libraries of nucleic acid nanostructure delivery compositions can be pooled and concentrated before administration to the animal of the nucleic acid barcode constructs associated with the nucleic acid nanostructure delivery compositions. Methods for library preparation and for sequencing are described in Green and Sambrook, “Molecular Cloning: A Laboratory Manual”, 4th Edition, Cold Spring Harbor Laboratory Press, (2012), incorporated herein by reference.

In various embodiments, cell or tissue samples may be analyzed for the presence of the nucleic acid nanostructure delivery compositions associated with the nucleic acid barcode constructs described herein. The samples can be any tissue, cell, or fluid sample from an animal, for example, selected from the group consisting of urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, lymph fluid, whole blood, serum, plasma, or any tissue or cell sample from an animal. Exemplary tissue or cell samples include brain tissue or cells, muscle tissue or cells, skin tissue or cells, heart tissue or cells, kidney tissue or cells, stomach tissue or cells, liver tissue or cells, urinary tract tissue or cells, gastrointestinal tract tissue or cells, head or neck tissue or cells, lung tissue or cells, reproductive tract tissue or cells, pancreatic tissue or cells, or any other tissue or cell type from an animal.

In one illustrative aspect for removing cells or tissues from the animal and isolating the nucleic acid barcode constructs from the cells or tissues of the animal, the nucleic acid barcode constructs are removed from cells or tissues of the animal. In various embodiments, nucleic acid barcode constructs (e.g., DNA or RNA) obtained from the tissues or cells of the animal can be removed by rupturing the cells and isolating the nucleic acid barcode constructs from the lysate. Techniques for rupturing cells and for isolation of nucleic acids are well-known in the art, and removal techniques include homogenization, such as by using a bead-beating technique. In other embodiments, the nucleic acid barcode constructs may be isolated by rupturing cells using a detergent or a solvent, such as phenol-chloroform. In another aspect, the nucleic acid barcode constructs may be separated from the lysate by physical methods including, but not limited to, centrifugation, dialysis, diafiltration, filtration, size exclusion, pressure techniques, digestion of proteins with Proteinase K, or by using a substance with an affinity for nucleic acids such as, for example, beads that bind nucleic acids.

In one illustrative embodiment, the nucleic acid barcode constructs are removed from cells or tissues by treating with a mixture of an organic phase (e.g., phenol chloroform) and an aqueous phase (e.g., water). The organic phase (e.g., phenol chloroform) is isolated and the nucleic acid barcode construct can be precipitated by raising the pH, for example, to pH 7.4. The organic phase (e.g., phenol chloroform) can be evaporated and the nucleic acid barcode constructs can be suspended in water and diluted to appropriate concentrations for PCR and/or sequencing. In one embodiment, the isolated nucleic acid barcode constructs are suspended in either water or a buffer after sufficient washing.

In other embodiments, commercial kits are available for isolation of the nucleic acid barcode constructs, such as Qiagen™, Nuclisensm™, Wizard™ (Promega), QiaAmp 96 DNA Extraction Kit™ and a Qiacube HT™ instrument, and Promegam™. Methods for preparing nucleic acids for PCR and/or sequencing are also described in Green and Sambrook, “Molecular Cloning: A Laboratory Manual”, 4th Edition, Cold Spring Harbor Laboratory Press, (2012), incorporated herein by reference.

The nucleic acid barcode constructs can be detected by using, for example, the polymerase chain reaction (PCR), isothermic amplification, sequencing, and/or imaging. The polymerase chain reaction (PCR) has been developed to analyze nucleic acids in a laboratory. PCR evolved over the last decade into a new generation of devices and methods known as Next Generation Sequencing (NGS). NGS provides faster detection and amplification of nucleic acids at a cheaper price. The NGS devices and methods allow for rapid sequencing as the nucleic acids are amplified in massively parallel, high-throughput platforms.

In one illustrative aspect, the nucleic acid barcode constructs can be sequenced, to detect the polynucleotide barcodes using any suitable sequencing method including Next Generation Sequencing (e.g., using Illumina, ThermoFisher, or PacBio or Oxford Nanopore Technologies sequencing platforms), sequencing by synthesis, pyrosequencing, nanopore sequencing, or modifications or combinations thereof can be used. In one embodiment, the sequencing can be amplicon sequencing. In another embodiment, the sequencing can be whole genome sequencing. In another embodiment, the sequencing can be exome/targeted hybridization sequencing. Methods for sequencing nucleic acids are also well-known in the art and are described in Sambrook et al., “Molecular Cloning: A Laboratory Manual”, Cold Spring Harbor Laboratory Press, incorporated herein by reference.

In another embodiment, a method of treating a patient with a disease is provided. The method comprises administering to the patient the nucleic acid nanostructure delivery composition identified in the in vivo screening method described herein, or administering to the patient any of the nucleic acid nanostructure delivery compositions described herein, wherein the nucleic acid nanostructure delivery compositions comprises a payload, and treating the disease in the patient.

Illustrative payloads for the nucleic acid nanostructure delivery compositions described herein can include any one or a combination of compositions selected from the group comprising: nucleic acids (e.g., DNA or RNA), pDNA, oligodeoxyribonucleic acids (ODNs), dsDNA, ssDNA, antisense oligonucleotides, antisense RNA, siRNA, messenger RNA, guide RNA (e.g., small guide RNA), ribonucleoproteins, donor DNA strands used in the CRISPR/Cas9 system, and enzymes, such as CRISPR-associated enzymes, e.g., Cas9, enzymes used in other gene editing systems, such as ZFNs, custom designed homing endonucleases, TALENS systems, other gene editing endonucleases, and reverse transcriptase.

Other illustrative payloads include DNA constructs such as chimeric antigen receptor (CAR) constructs. CAR-T cells are T cells expressing chimeric antigen receptors (CARs). The CAR is a genetically engineered receptor that is designed to target a specific antigen, for example, a tumor antigen. This targeting can result in cytotoxicity against the tumor, for example, such that CAR-T cells expressing CARs can target and kill tumors via the specific tumor antigens. CARs can comprise a recognition region, e.g., a single chain fragment variable (scFv) region derived from an antibody for recognition and binding to the antigen expressed by the tumor, an activation signaling domain, e.g., the CD3ζ chain of T cells can serve as a T cell activation signal in CARs, and a co-stimulation domain (e.g., CD137, CD28 or CD134) to achieve prolonged activation of T cells in vivo. In some aspects, CARs are large DNA constructs.

In another embodiment, the payload can be a nucleic acid (e.g., DNA or RNA) with a size selected from the group consisting of 3 kB or more, 3.1 kB or more, 3.2 kB or more, 3.3 kB or more, 3.4 kB or more, 3.5 kB or more, 3.6 kB or more, 3.7 kB or more, 3.8 kB or more, 3.9 kB or more, 4 kB or more, 4.1 kB or more, 4.2 kB or more, 4.3 kB or more, 4.4 kB or more, 4.5 kB or more, 4.6 kB or more, 4.7 kB or more, 4.8 kB or more, 4.9 kB or more, 5 kB or more, 5.1 kB or more, 5.2 kB or more, 5.3 kB or more, 5.4 kB or more, 5.5 kB or more, 5.6 kB or more, 5.7 kB or more, 5.8 kB or more, 5.9 kB or more, 6 kB or more, 6.1 kB or more, 6.2 kB or more, 6.3 kB or more, 6.4 kB or more, 6.5 kB or more, 6.6 kB or more, 6.7 kB or more, 6.8 kB or more, 6.9 kB or more, 7 kB or more, 7.1 kB or more, 7.2 kB or more, 7.3 kB or more, 7.4 kB or more, 7.5 kB or more, 7.6 kB or more, 7.7 kB or more, 7.8 kB or more, 7.9 kB or more, 8 kB or more, 8.1 kB or more, 8.2 kB or more, 8.3 kB or more, 8.4 kB or more, and 8.5 kB or more.

In various embodiments, the payload can be any one or more of the components of the CRISPR RNP system including a CRISPR-associated enzyme (e.g., Cas9), a short guide RNA (sgRNA), and a donor DNA strand. In an embodiment where the payload comprises Cas9, Cas9 can be fused to a deaminase. In yet another embodiment, the payload can comprise an sgRNA used for targeting an enzyme to a specific genomic sequence. In another aspect, the targeted enzyme can be a CRISPR-associated enzyme. In another illustrative aspect, the payload can comprise one molecule each of CRISPR/Cas9, an sgRNA, and a donor DNA strand in the nucleic acid nanostructure delivery compositions described herein. In another embodiment, the payloads can be nucleic acids used for homology directed repair or as transposable elements. In yet another embodiment, the payloads can be any of the payloads described herein in the form of a plasmid construct.

In one aspect, the nucleic acid nanostructure delivery composition described herein can encapsulate a payload that is used for gene editing. In one aspect, the CRISPR/Cas9 system can be the payload and can be used for gene editing. In another embodiment, another gene editing system can be the payload, such as ZFNs, custom designed homing endonucleases, and TALENS systems. In the embodiment where the CRISPR/Cas9 system is the payload, the Cas9 endonuclease is capable of introducing a double strand break into a DNA target sequence. In this aspect, the Cas9 endonuclease is guided by the guide polynucleotide (e.g., sgRNA) to recognize and optionally introduce a double strand break at a specific target site into the genome of a cell. In this illustrative embodiment, the Cas9 endonuclease can unwind the DNA duplex in close proximity to the genomic target site and can cleave both target DNA strands upon recognition of a target sequence by a guide polynucleotide (e.g., sgRNA), but only if the correct protospacer-adjacent motif (PAM) is approximately oriented at the 3′ end of the target. In this embodiment, the donor DNA strand can then be incorporated into the genomic target site. The CRISPR/Cas9 system for gene editing is well-known in the art.

In another illustrative embodiment, the payload may include DNA segments that serve as nuclear localization signals, enhancing nuclear delivery of the nucleic acid nanostructure delivery compositions upon endosomal escape. In another aspect, the payload may include a nucleotide sequence designed to bind as an aptamer to endosomal receptors, enhancing intracellular trafficking of the nucleic acid nanostructure delivery compositions.

In one illustrative aspect, a nucleic acid nanostructure delivery composition (e.g., DNA origami) is provided to package the Cas9 protein, the sgRNA and the single stranded donor DNA strand together in one nanostructure to ensure co-delivery of all the components to a particular location at the same time. In this embodiment, the single stranded nature of the sgRNA and the donor DNA strand can be used to convert these components into constitutive parts of the nucleic acid nanostructure delivery composition (e.g., the DNA origami structure) such that they get delivered together and dissociate at the same time from the DNA nanostructure delivery composition upon reaching the target site (e.g., a target cell). In this embodiment, the DNA nanostructure delivery composition can deliver either a plasmid or the ribonucleoprotein (RNP) form of CRISPR/Cas 9.

In one embodiment, a method for gene therapy is provided. In one aspect, the method comprises administering to a patient a nucleic acid nanostructure delivery composition described herein.

In one embodiment, the nucleic acid nanostructure delivery compositions described herein may be formulated as pharmaceutical compositions for parenteral or topical administration. Such pharmaceutical compositions and processes for making the same are known in the art for both humans and non-human mammals. See, e.g., REMINGTON: THE SCIENCE AND PRACTICE OF PHARMACY, (1995) A. Gennaro, et al., eds., 19th ed., Mack Publishing Co. Additional active ingredients may be included in the compositions.

In one aspect, the nucleic acid nanostructure delivery composition may be administered, for example, directly into the blood stream of a patient, into muscle, into an internal organ, or can be administered in a topical formulation. In various embodiments, suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but they may be more suitably formulated as a sterile non-aqueous solution or as a dried form to be used in conjunction with a suitable vehicle such as sterile, pyrogen-free water or sterile saline. The preparation under sterile conditions, by lyophilization to produce a sterile lyophilized powder for a parenteral formulation, may readily be accomplished using standard pharmaceutical techniques well-known to those skilled in the art. In one embodiment, the solubility of the composition used in the preparation of a parenteral formulation may be increased by the use of appropriate formulation techniques, such as the incorporation of solubility-enhancing agents.

In one illustrative embodiment, pharmaceutical compositions for parenteral administration comprise: a) a pharmaceutically active amount of the nucleic acid nanostructure delivery composition; b) a pharmaceutically acceptable pH buffering agent to provide a pH in the range of about pH 4.5 to about pH 9; c) an ionic strength modifying agent in the concentration range of about 0 to about 300 millimolar; and d) water soluble viscosity modifying agent in the concentration range of about 0.25% to about 10% total formula weight or any combinations of a), b), c) and d) are provided.

In various illustrative embodiments, the pH buffering agents for use in the compositions and methods described herein are those agents known to the skilled artisan and include, for example, acetate, borate, carbonate, citrate, and phosphate buffers, as well as hydrochloric acid, sodium hydroxide, magnesium oxide, monopotassium phosphate, bicarbonate, ammonia, carbonic acid, hydrochloric acid, sodium citrate, citric acid, acetic acid, disodium hydrogen phosphate, borax, boric acid, sodium hydroxide, diethyl barbituric acid, and proteins, as well as various biological buffers, for example, TAPS, Bicine, Tris, Tricine, HEPES, TES, MOPS, PIPES, cacodylate, or MES.

In another illustrative embodiment, the ionic strength modulating agents include those agents known in the art, for example, glycerin, propylene glycol, mannitol, glucose, dextrose, sorbitol, sodium chloride, potassium chloride, and other electrolytes.

Useful viscosity modulating agents include but are not limited to, ionic and nonionic water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; gums such as tragacanth and xanthan gum; sodium alginate; gelatin, hyaluronic acid and salts thereof, chitosans, gellans or any combination thereof. Typically, non-acidic viscosity enhancing agents, such as a neutral or a basic agent are employed in order to facilitate achieving the desired pH of the formulation.

In one embodiment, the solubility of the compositions described herein used in the preparation of a parenteral formulation may be increased by the use of appropriate formulation techniques, such as the incorporation of solubility-enhancing agents.

In other embodiments, the compositions described herein may be administered topically. A variety of dose forms and bases can be applied to the topical preparations, such as an ointment, cream, gel, gel ointment, plaster (e.g. cataplasm, poultice), solution, powders, and the like. These preparations may be prepared by any conventional method with conventional pharmaceutically acceptable carriers or diluents as described below.

For example, vaseline, higher alcohols, beeswax, vegetable oils, polyethylene glycol, etc. can be used. In the preparation of a cream formulation, fats and oils, waxes, higher fatty acids, higher alcohols, fatty acid esters, purified water, emulsifying agents etc. can be used. In the preparation of gel formulations, conventional gelling materials such as polyacrylates (e.g. sodium polyacrylate), hydroxypropyl cellulose, hydroxypropyl methyl cellulose, polyvinyl alcohol, polyvinylpyrrolidone, purified water, lower alcohols, polyhydric alcohols, polyethylene glycol, and the like are used. In the preparation of a gel ointment, an emulsifying agent (preferably nonionic surfactants), an oily substance (e.g. liquid paraffin, triglycerides, and the like), etc. are used in addition to the gelling materials as mentioned above. A plaster such as cataplasm or poultice can be prepared by spreading a gel preparation as mentioned above onto a support (e.g. fabrics, non-woven fabrics). In addition to the above-mentioned ingredients, paraffins, squalane, lanolin, cholesterol esters, higher fatty acid esters, and the like may optionally be used. Moreover, antioxidants such as BHA, BHT, propyl gallate, pyrogallol, tocopherol, etc. may also be incorporated. In addition to the above-mentioned preparations and components, there may optionally be used any other conventional formulations for incorporation with any other additives.

In various embodiments, the dosage of the nucleic acid nanostructure delivery composition can vary significantly depending on the patient condition, or the disease state being treated, the route of administration and tissue distribution, and the possibility of co-usage of other therapeutic treatments. The effective amount to be administered to a patient is based on body surface area, patient weight or mass, and physician assessment of patient condition. In various embodiments, the nucleic acid nanostructure delivery composition can be administered to a patient with a disease or a disorder selected from the group consisting of cancer, a muscular disorder, a pulmonary disorder, a skin disorder, a neurological disease, neurofibromatosis 1 (NF1), and a hemoglobinopathy. In one embodiment, the cancer is selected from the group consisting of lung cancer, bone cancer, pancreatic cancer, skin cancer, uterine cancer, ovarian cancer, endometrial cancer, rectal cancer, stomach cancer, colon cancer, breast cancer, cancer of the esophagus, cancer of the endocrine system, prostate cancer, leukemia, lymphoma, mesothelioma, cancer of the bladder, cancer of the kidney, neoplasms of the central nervous system, brain cancer, and adenocarcinoma. In another embodiment, the skin disorder is a Staphylococcus aureus infection. In yet another embodiment, the muscular disorder is muscular dystrophy (e.g., Duchenne Muscular Dystrophy). In still another embodiment, the nucleic acid nanostructure delivery composition are not cytotoxic to the cells of the patient. In another embodiment, the gene therapy may result in the inactivation of a pathogen (i.e., a microorganism) rather than altering the genome of the patient.

In another embodiment, a method is provided comprising synthesizing a diverse set of non-viral gene delivery compositions, wherein each non-viral gene delivery composition differs from each other non-viral gene delivery composition of the diverse set with respect to at least one of a set of composition characteristics, simultaneously testing one or more quality attributes of each of the non-viral gene delivery composition of the diverse set, and creating from results of the testing, a predictive model that correlates the composition characteristics with the quality attributes. In this embodiment, the composition characteristics can comprise one or more of molecular weight, degree of branching, number of ionizable groups, core-to-corona molecular weight ratio, hydrophilicity, hydrophobicity, propensity for aggregation, size, pKa, logP, and surface charge. In this embodiment, the quality attributes can comprise one or more of cytotoxicity, immunogenicity, transfection efficiency, zeta potential, size, pKa, logP, and loading efficiency. In this embodiment, the diverse set can comprises hundreds or thousands of non-viral gene delivery compositions. In this embodiment, each of the non-viral gene delivery compositions of the diverse set can be a nucleic acid nanostructure delivery composition according to any one of clauses described above. In one embodiment of this method, high-throughput testing, and machine learning data analysis can accelerate the design-build-test-learn (DBTL) cycle for development of CRISPR-based therapeutics.

In some embodiments, the nucleic acid nanostructure delivery composition may be labelled to enhance downstream separation. For example, this may include covalently bonding the nucleic acid nanostructure delivery composition to a magnetic nanoparticle (e.g., superparamagnetic iron oxide), to polyhistidine tags for metal ion chromatography, and/or to fluorescent labels for fluorescent assisted separation (such as with FACS). The labels may be used to track the nucleic acid delivery composition in vivo. Possible “endpoints” include, but are not limited to, quantitative presence in various physiological tissue, post administration, measured via, for example, fluorescence.

In the embodiment where labels are used, the labels allow for rapid in vivo screening of many non-viral delivery vehicle variants with parallel determination of quantitative bio-distribution, and rapid in vitro screening of many variants for stability, cytotoxicity, immunogenicity, and efficacy. This embodiment allows for the construction of a large library of non-viral delivery vehicles that can be drawn from for use as delivery vehicles for genetic medicines, including gene therapies, genetic vaccines, gene editing, gene regulators, and small molecule therapeutics.

In some illustrative embodiments, a large library of similar, but unique nucleic acid nanostructure delivery compositions may be constructed for use in a high-throughput screening process to identify targeting components that bind to specific targets. This rapid screening platform can quickly determine an effective targeting molecule that can be used for targeted delivery to a specific cell or tissue, or for use as a neutralizing molecule for a pathogen.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

While certain illustrative embodiments have been described in detail in the drawings and the foregoing description, such an illustration and description is to be considered as exemplary and not restrictive in character, it being understood that only illustrative embodiments have been shown and described and that all changes and modifications that come within the spirit of the disclosure are desired to be protected. There exist a plurality of advantages of the present disclosure arising from the various features of the apparatus, systems, and methods described herein. It will be noted that alternative embodiments of the apparatus, systems, and methods of the present disclosure may not include all of the features described, yet still benefit from at least some of the advantages of such features. Those of ordinary skill in the art may readily devise their own implementations of the apparatus, systems, and methods that incorporate one or more of the features of the present disclosure.

While the concepts of the present disclosure are susceptible to various modifications and alternative forms, specific embodiments thereof have been shown by way of example in the appended drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure.

### Example 1

### Nucleic Acid Nanostructure Delivery Compositions

The materials and methods below describe an example in which a DNA origami (DNAO) nanostructure is a cuboid structure and the nucleic acid barcode constructs are attached to the DNAO via complementary base pairing of the barcodes with one of the oligonucleotide staples within the DNAO.

### Barcode Design

The barcodes used in this example comprise a unique portion comprising 8 to 10 nucleotides in the center of the polynucleotide, the unique portion further characterized by a hamming distance of at least 3 bases from any other barcodes to be pooled. Directly on the 3′ end of the barcode, 7 to 10 random bases are included for bioinformatic removal of PCR duplicates. This central sequence is flanked by universal primer annealing sites containing overhangs for the addition of index adapters during sequencing library preparation. The polynucleotide barcodes in this example were designed with a biotin functional group on the 5′ end.

(SEQ ID NO: 1004)
/5BiosG/A*G*A*CGTGTGCTCTTCCGATCTGAGGGTACTTNNNNNNN
NNNAGATCGGAAGAGCGTCG*T*G*T.

### Materials for Nucleic Acid Nanostructure Delivery Composition Synthesis (DNAO)

The DNA origami scaffold is a single stranded DNA (ssDNA) isolated from the M13 bacteriophage. The oligonucleotide staples are short single stranded DNA with sequences described in Table 2. The barcode is a single stranded DNA segment as described under barcode design above.

### Preparation of the DNA Barcoded DNAO Structures

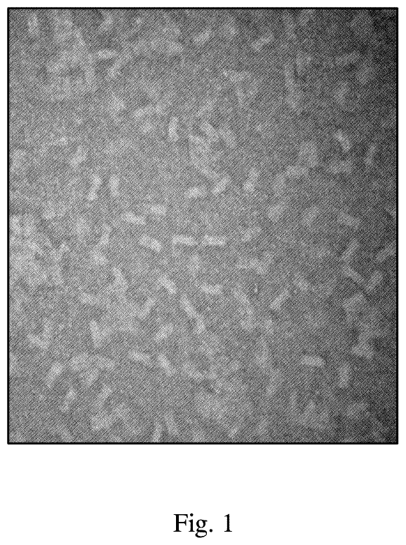

A reaction mixture was prepared comprising the DNA scaffold, the oligonucleotide staples and magnesium together in TE buffer in a reaction vessel in the following amounts: 160 uL all oligonucleotide staples (described in Table 2) pooled at a total concentration of 500 nM, 80 uL scaffold (100 nM), 80 uL water, 40 uL of TE buffer (1.46 g EDTA, 3.03 g Tris Base, 1.46 g NaCl, 500 mL water), 40 uL of 200 mM MgCl₂. The reaction vessel was placed in a thermocycler with thermal ramp starting at ˜65° C. and descending to 24° C. over the course of ˜67 hours. The product was purified using a precipitation with a PEG purification protocol using a PEG solution made with the following recipe: 75 g PEG8000, 50 mL of the TE buffer described above, and 62.5 mL of 4 M NaCl, brought up to 500 mL with water, yielding a DNAO nanostructure in water. The concentration was measured via nanodrop and then the barcodes were added to the product at a molar ratio of 4:1 (polynucleotide barcodes to DNAO nanostructure). This mixture was incubated at 37° C. for 2 to 3 hours. The product was purified with another PEG purification process as described above, yielding a final product of DNA barcoded DNAO in water. Transmission electron microscopy images were captured on formvar/carbon coated nickel grids with a negative stain with 1% phosphotungstic acid (PTA) using an FEI Tecnai G2 Bio Twin TEM (see FIG. 1).

### Barcode Amplification

Dilutions of the DNA barcoded DNAO nanostructure described above were prepared at the following concentrations: 13.5, 1.35, 0.135 and 0.0135 nM. A Master Mix was created with: Kapa HiFi 2x Master mix, Reverse barcode primer, forward barcode primer, DMSO, and nuclease free water. Master Mix (15 μL) was loaded into each well. Each of the dilutions of DNA barcoded DNAO was loaded (5 μL) into each well of a 96 well plate. Nuclease free water (5 μL) was loaded into the designated NTC wells. Positive control (5 μL) was loaded into the designated positive control wells where the positive control comprised a solution of polymer nanoparticles in phosphate buffered saline consisting of dimethylaminoethylmethacrylate, polyacylate, and butyl methacrylate, labelled with the same barcode as that used to label the DNAO, as described in U.S. patent application Ser. No. 17/715,784. Each well was covered, either with a strip cap or adhesive seal, and centrifuged for approx. 1 min at 1,000×g. Amplification of the barcodes was conducted by incubating in a thermocycler under typical PCR conditions.

### Analysis of PCR Amplification Via Gel Electrophoresis

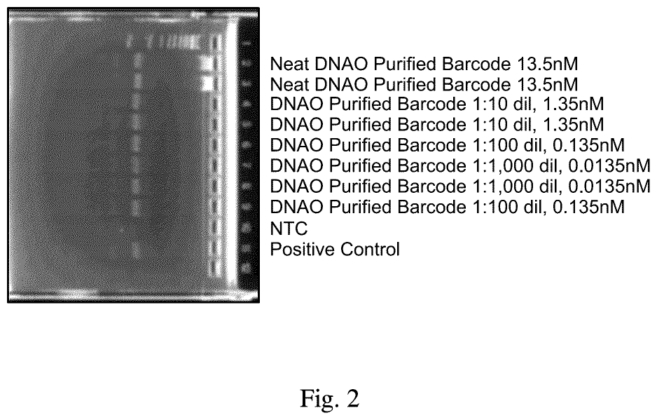

The gel electrophoresis was done on a 4% 12-well Ethidium Bromide gel, using 15 μL of 1 kb E-gel Ladder in the first well. DNA barcoded DNAO dilutions (10 μL) from the multiwell plate above were added to each well of the 12-well gel. E-gel buffer (10 μL) was added to each well. Nuclease free water (15 μL) was added to any remaining empty wells as the no test control (NTC). The gel doc was powered on to run current through the gel for about 20 to 25 mins, or until the sample buffer line reached the end of the gel. The gel was removed from the base and analyzes in a Gel Imager (see FIG. 2).

### Results

FIG. 1 shows a transmission electron micrograph of the DNA barcoded DNAO nanostructures. The image shows evidence that the nanostructures were successfully folded into cuboid nanostructures, showing that the DNA origami folding process was successful. The TEM does not offer the resolution to discern the polynucleotide barcodes on the structure, therefore, PCR amplification was used prove their presence as shown in FIG. 2.

TABLE 2

| | | |
|---|---|---|
| Cuboid Body-1 | ACTGAGTGACTTCACATGGAGCGGTATGGTT | (SEQ ID |
| | | NO: 1005) |
| Cuboid Body-2 | TACGGCCTCTAAACGACAATCGGCACTCCAA | (SEQ ID |
| | | NO: 1006) |
| Cuboid Body-3 | GACCATACCGGAAGCAAACTATAATACTGATATTC | (SEQ ID |
| | | NO: 1007) |
| Cuboid Body-4 | CCGTGACCAGGTCATTACCATTGAACGAGAAT | (SEQ ID |
| | | NO: 1008) |
| Cuboid Body-5 | ACACCTACGAAAAAACAAAATTAAT | (SEQ ID |
| | | NO: 1009) |
| Cuboid Body-6 | ATTCAGAGCATAAATAGCTAT | (SEQ ID |
| | | NO: 1010) |
| Cuboid Body-7 | TAACCACAGAAACTTTCATCAGTATCTTTTC | (SEQ ID |
| | | NO: 1011) |
| Cuboid Body-8 | GACGGACTCCTATAACCCACAAGAAACAAAGTCCTAATT | (SEQ ID |
| | | NO: 1012) |
| Cuboid Body-9 | AGCGAAAAATGCCATCATCTTTGATTGTAAAAGTT | (SEQ ID |
| | | NO: 1013) |
| Cuboid Body-10 | GAACCCTAAGGATATTTAATTCCTGACTATAGCGT | (SEQ ID |
| | | NO: 1014) |
| Cuboid Body-11 | CCGCGATTAGGACGCAGTTTGACGG | (SEQ ID |
| | | NO: 1015) |
| Cuboid Body-12 | TAAGGTTTAGTCATAGTTCATGTAGATACCGAAGGCTTG | (SEQ ID |
| | | NO: 1016) |
| Cuboid Body-13 | AATATTTGACCCTGAATCATACAGAAAATCCCCCT | (SEQ ID |
| | | NO: 1017) |
| Cuboid Body-14 | CCAATACGCAGACGACGATAAACCAACTAGAAACCGATTATC | (SEQ ID |
| | | NO: 1018) |
| Cuboid Body-15 | CAGGCTGAAAGTACAAATTCGTGAAAGC | (SEQ ID |
| | | NO: 1019) |
| Cuboid Body-16 | TACATCCAAGACGCGCCCCCTCAGC | (SEQ ID |
| | | NO: 1020) |
| Cuboid Body-17 | AATACAGTAGGGCAAGCCGAAACATGGCAACAAAAAGGG | (SEQ ID |
| | | NO: 1021) |
| Cuboid Body-18 | ATTAGCGACCAGAGCCACCCT | (SEQ ID |
| | | NO: 1022) |
| Cuboid Body-19 | AAGAAATTTTAATTTATCGCGCATT | (SEQ ID |
| | | NO: 1023) |
| Cuboid Body-20 | CAGGGAGGAGAAACACAGTAACCTACCAGACAACT | (SEQ ID |
| | | NO: 1024) |
| Cuboid Body-21 | CCCACCTCCTTACCGAGATTTTTT | (SEQ ID |
| | | NO: 1025) |
| Cuboid Body-22 | CACCAGAGCCGCAGCAGAGGG | (SEQ ID |
| | | NO: 1026) |
| Cuboid Body-23 | GGCCAAATCCCAGCGATCGGGTAAAACAATT | (SEQ ID |
| | | NO: 1027) |
| Cuboid Body-24 | TGCGGGAACCTAAAACGAATTGATGATG | (SEQ ID |
| | | NO: 1028) |
| Cuboid Body-25 | ACCCTGAGTTTCACAAACAAAAGAAAGCCCA | (SEQ ID |
| | | NO: 1029) |
| Cuboid Body-26 | GCATTGACAGGACAAATTGTA | (SEQ ID |
| | | NO: 1030) |
| Cuboid Body-27 | GAGCGGGATCCTGAAAAGTGT | (SEQ ID |
| | | NO: 1031) |
| Cuboid Body-28 | CATTAGTTGATGCCAGCTGTAATGATTGCCCTAGAATCA | (SEQ ID |
| | | NO: 1032) |
| Cuboid Body-29 | ATATCGCATAGTAATTTTTTGGATT | (SEQ ID |
| | | NO: 1033) |
| Cuboid Body-30 | GCGTAAAATTTAAGGGTGAGAAAGG | (SEQ ID |
| | | NO: 1034) |
| Cuboid Body-31 | TAATGTTAGCACTAGAAGGTTCATCACCCAACAGT | (SEQ ID |
| | | NO: 1035) |
| Cuboid Body-32 | AGAGCCATTCGGATGTTTTTGCCCAATCAAG | (SEQ ID |
| | | NO: 1036) |
| Cuboid Body-33 | GCTTTGAAAGAGTCTTATCCG | (SEQ ID |
| | | NO: 1037) |
| Cuboid Body-34 | CAGTCGCGTCGCTTGTTGGGGTGCAACGGTA | (SEQ ID |
| | | NO: 1038) |
| Cuboid Body-35 | GTTATACAAAACATAACGCCAAAAG | (SEQ ID |
| | | NO: 1039) |
| Cuboid Body-36 | GAACTGGCTCAACAAAAGGTAAAGTAA | (SEQ ID |
| | | NO: 1040) |
| Cuboid Body-37 | AAGCGGGCGATCACTCCATCCCAATGTATAAGGCTATTA | (SEQ ID |
| | | NO: 1041) |
| Cuboid Body-38 | AGATGATAGCGCGATGTAAATTCGAGAAAGGAACCTTA | (SEQ ID |
| | | NO: 1042) |
| Cuboid Body-39 | CGTCAAATCATTAAGAAACCATCGATTTATTAGCGGCC | (SEQ ID |
| | | NO: 1043) |
| Cuboid Body-40 | CAGAGGCTTTGAGGACCTTTAGATTAGCGAACTGCTATTAAACGA | (SEQ ID |
| | TGAATAACA | NO: 1044) |
| Cuboid Body-41 | TTACCTCGAGATTTCGCTCGCTGAATTCAGAAAGCGGTGCCGTA | (SEQ ID |
| | | NO: 1045) |
| Cuboid Body-42 | AAAAGAAACATCGGTTAAAGGTCTTAAAAGCCTTTGTTTCAGGCC | (SEQ ID |
| | | NO: 1046) |
| Cuboid Body-43 | ATACTTCATACACTAGAAAACTTATCATAACTACATCA | (SEQ ID |
| | | NO: 1047) |
| Cuboid Body-44 | CGACATTGGGTCGAGAGGTCCACGCTGGCTTCTAATTA | (SEQ ID |
| | | NO: 1048) |
| Cuboid Body-45 | TAAGTTGCACCGAAAGCGTGTACTGGTAATATCAAGAGT | (SEQ ID |
| | | NO: 1049) |
| Cuboid Body-46 | AAAAAAAGGAAAGAGCCACCACCACCGGAACCGTCATAGTTA | (SEQ ID |
| | | NO: 1050) |
| Cuboid Body-47 | TACATTTAATACGTGACAGCAGTAGGAATATAGAAAACGCTATAC | (SEQ ID |
| | AAAAACTGAACA | NO: 1051) |
| Cuboid Body-48 | AAATTATGGGCGAAGATGGTGCAGCTGAGTAAACATAAAGACCC | (SEQ ID |
| | ATCAAAA | NO: 1052) |
| Cuboid Body-49 | TGCCAGTACGAGCGTTGATATTCGTCACAGAGCCATTT | (SEQ ID |
| | | NO: 1053) |
| Cuboid Body-50 | TACGAGCCGCCCCGGGGGTTGGTTTCCGGCGCAACTGAAC | (SEQ ID |
| | | NO: 1054) |
| Cuboid Body-51 | GTCTTTACGGTTGTAATTAGCTGAGAGCCAGCATAAACAGTTCAG | (SEQ ID |
| | GCAATAG | NO: 1055) |
| Cuboid Body-52 | TTTAAAGGTGGAGCAATAAAGCCTACCGCCTGTTGCTG | (SEQ ID |
| | | NO: 1056) |
| Cuboid Body-53 | CCCTGATTGAGTTACTGGCACAAACGTCTCCTCACCAGGGTAGGCT | (SEQ ID |
| | GCACC | NO: 1057) |
| Cuboid Body-54 | CCGGTATTTTTTATGCACTCAGCTGATGGTTAAATAGGACAGCAAA | (SEQ ID |
| | GCTTGCGGAAAATC | NO: 1058) |
| Cuboid Body-55 | AGTTCTGGTGCAACTCTGTGAGAGAGTATAACGTGCTTTATCAGTG | (SEQ ID |
| | CAACA | NO: 1059) |
| Cuboid Body-56 | AATAAGAGGCACTGACCTAATATATTTAGAGGCAAAAGATGAATA | (SEQ ID |
| | ATGCCCGAACG | NO: 1060) |
| Cuboid Body-57 | AAAGCCTACCTCGAGGGCTTAAAGATCGCGGTGCGGTT | (SEQ ID |
| | | NO: 1061) |
| Cuboid Body-58 | CTGGACCTACATTTTGACGCTCATTATAGCTGTTTCCCCGAC | (SEQ ID |
| | GCATTCT | NO: 1062) |
| Cuboid Body-59 | GCGTCCATTACGAGAGGGCCAGGGTAAAGGGGCAG | (SEQ ID |
| | | NO: 1063) |
| Cuboid Body-60 | CCGGAGATCAACGCTCTGACCCAATCGTCTGAAATTTAAATATGCT | (SEQ ID |
| | CCTCCCTCGT | NO: 1064) |
| Cuboid Body-61 | CGCCAGAAGCTAAACGGGCAAGTTCCGAGGCCAGTGGGTITTAAC | (SEQ ID |
| | CGCCTAG | NO: 1065) |
| Cuboid Body-62 | AATAACAATAAAGAGCCGCAACAGTATATCAAGGT | (SEQ ID |
| | | NO: 1066) |
| Cuboid Body-63 | GTAAGAAGAGAAGCAACCAGAAATCAAATCGTCAT | (SEQ ID |
| | | NO: 1067) |
| Cuboid Body-64 | ATTCTATCAAAATTATTTCGTCAGAGTTTATCTTACGAGAGC | (SEQ ID |
| | | NO: 1068) |
| Cuboid Body-65 | TTCGAGTCTTTCCTTTTTCAAAATTTAGACCTTCATCAAATAATCCTT | (SEQ ID |
| | TGGAAGGG | NO: 1069) |
| Cuboid Body-66 | GTACGCGCTAGGAAGGGAATGCGCATTATTCTCAAAGTITTAACG | (SEQ ID |
| | GAAG | NO: 1070) |
| Cuboid Body-67 | ACCATCAAAATCACCGGATTTGCCAGCGACAGGAAACGTACCAGG | (SEQ ID |
| | CCGTTGTATCG | NO: 1071) |
| Cuboid Body-68 | AGGGATTAACCAACTATCGGTGTGTGAAATTGTGTCCATCACGCA | (SEQ ID |
| | ACGC | NO: 1072) |
| Cuboid Body-69 | AAATAGAGGTCTAGCTCAAGTGCGGGCCTTGAGCAAGTGGCGCC | (SEQ ID |
| | GCTAC | NO: 1073) |
| Cuboid Body-70 | AGAGCATTTTCCGTAACAAAGTACCTTTCATCTCGGAACGA | (SEQ ID |
| | | NO: 1074) |
| Cuboid Body-71 | CTACACGCGAGGCGTTTTAAGACGCTGAGAAGCTA | (SEQ ID |
| | | NO: 1075) |
| Cuboid Body-72 | AACCTCAATGAAAGGCTTTAATAGTCAGGGCTGGCGTTTCCCAATC | (SEQ ID |
| | AAAAGTC | NO: 1076) |
| Cuboid Body-73 | CGTATTACTTTAGGTCATCTTTTTTCGACCCTCAGGCTCAGTCACCA | (SEQ ID |
| | ATAGGTGAA | NO: 1077) |
| Cuboid Body-74 | TGAGTAAGTGTACAGTGATAACGCCATACCACCCTGCTGAGAAGA | (SEQ ID |
| | AAATTTGAGG | NO: 1078) |
| Cuboid Body-75 | CACTATTAAAGCGTCACCAAGGCCGAATCAAGGGATTTTAGAATA | (SEQ ID |
| | GAAGGCTCCAA | NO: 1079) |
| Cuboid Body-76 | AAATTAATATCGAACTCAGTTGTAGCAATACTGCTTAATTAGCGGT | (SEQ ID |
| | CCGGCGATTA | NO: 1080) |
| Cuboid Body-77 | CCTGTAGTCAGAAATTTGACCAGCTGAAATTTCAAAATTGAGACA | (SEQ ID |
| | ACTAACA | NO: 1081) |
| Cuboid Body-78 | ATAATATCAGAGAGTAAGCAGCACCGTAAAGGTTGAGGCAGGAC | (SEQ ID |
| | GCCTGACC | NO: 1082) |
| Cuboid Body-79 | AACCAATTTAAATTATATAACAACATCCGAAACACCGG | (SEQ ID |
| | | NO: 1083) |
| Cuboid Body-80 | AAACAAACATCAAGGGCACCATCGTCACAACTTGCT | (SEQ ID |
| | | NO: 1084) |
| Cuboid Body-81 | TCATTTGAATTACCAAGTTTCGCAACGGCAAGCCGTCT | (SEQ ID |
| | | NO: 1085) |
| Cuboid Body-82 | GAGACAGGAGGCCGATTAAAGGGCTTTTTTCTCTTATAAGTCAC | (SEQ ID |
| | GAGGA | NO: 1086) |
| Cuboid Body-83 | GATAGCTTAATATCAGTTATTCAGTAGAGTACCAAAGCGCTTTAT | (SEQ ID |
| | TCAGTCAAATCACCA | NO: 1087) |
| Cuboid Body-84 | TAAAAACAATAGCAGGAAACGAAACGCGAACCTACGACTTATT | (SEQ ID |
| | CTAATTAC | NO: 1088) |
| Cuboid Body-85 | CTATGTATCGGGATTTAGAGCCCCAAACTGATAGCCCTA | (SEQ ID |
| | | NO: 1089) |
| Cuboid Body-86 | ATAATGAAGATTTCTGCGATCTACTAAATTGGGATGAGTAATCT | (SEQ ID |
| | TGATTGCAAATGAA | NO: 1090) |
| Cuboid Body-87 | TTATCACAACGTGGAAAATCCTTTCACCTGGACCGTAATGGGGA | (SEQ ID |
| | CGACATAC | NO: 1091) |
| Cuboid Body-88 | AAACACGTTGATAACCAGCTTTGGGAACAACTCACTCGT | (SEQ ID |
| | | NO: 1092) |
| Cuboid Body-89 | AAGCACAAAGACTATAAAACGAGGAACAGAATGCAG | (SEQ ID |
| | | NO: 1093) |
| Cuboid Body-90 | GGGTACATTAAATATACCAGGCAATTATTITGCGGAACAATTG | (SEQ ID |
| | AAAGAAG | NO: 1094) |
| Cuboid Body-91 | GTGTTITTATACCTCGTTTCACCGCCGAAAATGGA | (SEQ ID |
| | | NO: 1095) |
| Cuboid Body-92 | AACATTATTACCTGACGAAATCCAACAGGTCAGGA | (SEQ ID |
| | | NO: 1096) |
| Cuboid Body-93 | CAGCGCCTAAATCGAGAAAGGGCGCTGATCGGCTTTG | (SEQ ID |
| | | NO: 1097) |
| Cuboid Body-94 | AGACGGGGAGCAAGAATAACGTAAAGGTGAAAGTAGTGGTTG | (SEQ ID |
| | CCAAAGCGC | NO: 1098) |
| Cuboid Body-95 | CAAATGCCAAGAACCAGAACGCTAACGGTTGGGAATTTCTCACC | (SEQ ID |
| | TGTTTAAACCGTCT | NO: 1099) |
| Cuboid Body-96 | ATCAAGTAAATAATGTTAGTGATTAAATTGGCCTCTTTCCGCAA | (SEQ ID |
| | GCAAAT | NO: 1100) |
| Cuboid Body-97 | CAATAGTGTTATATGAATCATCGAACTGAAACCAAAGACTGGAT | (SEQ ID |
| | TATAGTCAGAAGTTC | NO: 1101) |
| Cuboid Body-98 | ATAATAAAGAATTATAAACAGCCATATTTCCTGAAATCCTCACA | (SEQ ID |
| | TGTACAGG | NO: 1102) |
| Cuboid Body-99 | TTAAATTTGTTAGGTGTCTGGATTAGGAATAAGAACGTAAATGA | (SEQ ID |
| | ACGCATTATCCATC | NO: 1103) |
| Cuboid Body-100 | CAGAGCCCGGAGTGGCTAAACCAGCCCTACCGCCAGCCAGTAG | (SEQ ID |
| | AAGAAATGG | NO: 1104) |
| Cuboid Body-101 | GCCACGCAAAATTACATCAATACGAGTACCTCAGGAGCTACGAGT | (SEQ ID |
| | | NO: 1105) |
| Cuboid Body-102 | AAGCACGCAATAAACAATGAAATAGCAGGGAACCAATCCAAA | (SEQ ID |
| | | NO: 1106) |
| Cuboid Body-103 | CTCATTTAGGCAAACGAAAGTAGTAATAGTAGGGCAAAGACCAA | (SEQ ID |
| | AAATAAATTAATGCCG | NO: 1107) |
| Cuboid Body-104 | CAGTATTAACAAAAATACGGGCGCGATTAGATAGGGGACATAGG | (SEQ ID |
| | TCAGGGGGGCA | NO: 1108) |
| Cuboid Body-105 | CGAGTAACGAGCACGTTGCAGCAAGCAAAAATCATCATGGT | (SEQ ID |
| | CAAATTTTTG | NO: 1109) |
| Cuboid Body-106 | AAATATTCCAAATCGCTTGCCAGGT | (SEQ ID |
| | | NO: 1110) |
| Cuboid Body-107 | TTATGCTGGCTATGGTTTCATGTAAGAA | (SEQ ID |
| | | NO: 1111) |
| Cuboid Body-108 | GAGGGAAGTCACCCACAGCAGGCTGG | (SEQ ID |
| | | NO: 1112) |
| Cuboid Body-109 | AATATAATCCTGACCCCAATCGCATTAA | (SEQ ID |
| | | NO: 1113) |
| Cuboid Body-110 | GCTTTCATTCCGTAAACGCACAGAC | (SEQ ID |
| | | NO: 1114) |
| Cuboid Body-111 | CCAACCACATTATCATAATTTGATAATC | (SEQ ID |
| | | NO: 1115) |
| Cuboid Body-112 | AACAACGCCAAGAAATACGAACGCGAAA | (SEQ ID |
| | | NO: 1116) |
| Cuboid Body-113 | GAGAGGGGCTAAATATGCGCGAAAA | (SEQ ID |
| | | NO: 1117) |
| Cuboid Body-114 | TCAATATTTTGCGGTACGTGGTTAA | (SEQ ID |
| | | NO: 1118) |
| Cuboid Body-115 | CATTCAAGATTCTGAGAATATAA | (SEQ ID |
| | | NO: 1119) |
| Cuboid Body-116 | GTAACTGTATGTTTGCCTCCCCCTT | (SEQ ID |
| | | NO: 1120) |
| Cuboid Body-117 | TTAGAACAGTACCTTTAGTTTCAA | (SEQ ID |
| | | NO: 1121) |
| Cuboid Body-118 | AAATCTAAAGATCTAAACTTGAGAAATC | (SEQ ID |
| | | NO: 1122) |
| Cuboid Body-119 | CGACACATGTTGGATTATAGAGATA | (SEQ ID |
| | | NO: 1123) |
| Cuboid Body-120 | GCCAACGTTGGATAAGAGGTTCAGC | (SEQ ID |
| | | NO: 1124) |
| Cuboid Body-121 | TCGGTTTATCAGTAAGCGCCA | (SEQ ID |
| | | NO: 1125) |
| Cuboid Body-122 | CAGATCATTACACGGGTAAGACAAACGA | (SEQ ID |
| | | NO: 1126) |
| Cuboid Body-123 | CATTTCGGCTGGTGAATTCCGCTTT | (SEQ ID |
| | | NO: 1127) |
| Cuboid Body-124 | AAGGCAGCTTGAAACCAAAACAGAA | (SEQ ID |
| | | NO: 1128) |
| Cuboid Body-125 | GAAGAGAGAAGCACCCTCCAG | (SEQ ID |
| | | NO: 1129) |
| Cuboid Body-126 | TAATTGAGCGCTAAGAACCAC | (SEQ ID |
| | | NO: 1130) |
| Cuboid Body-127 | TTTTGAGAGATCTAATTCGCC | (SEQ ID |
| | | NO: 1131) |
| Cuboid Body-128 | GCCAGATTTTAGACAGGCTAATGA | (SEQ ID |
| | | NO: 1132) |
| Cuboid Body-129 | AACCGTTCTAGCTGACATTATTTGAATGCAA | (SEQ ID |
| | | NO: 1133) |
| Cuboid Body-130 | ATATAGGAACGGGAGGATGAAGCATGAA | (SEQ ID |
| | | NO: 1134) |
| Cuboid Body-131 | AGGTCTTTACCGAGCTTCTTTAATTGCA | (SEQ ID |
| | | NO: 1135) |
| Cuboid Body-132 | CTCACAATCGTAATTTCTCCGCGGAAAC | (SEQ ID |
| | | NO: 1136) |
| Cuboid Body-133 | TTTTTTGCAACCGAACATACAGAAT | (SEQ ID |
| | | NO: 1137) |
| Right End-PolyT 5-1 | TTTTTGTAACCGTGCATCTGCGATGTGCTGCAAGGCGTTTTT | (SEQ ID |
| | | NO: 1138) |
| Right End-PolyT 5-2 | TTTTTCCCACGCATAACCGATATATTCGCTG | (SEQ ID |
| | | NO: 1139) |
| Right End-PolyT 5-3 | TTTTTAGGCGGTTTGCGTATTCCGAGATAGGGTTGAGTTTTT | (SEQ ID |
| | | NO: 1140) |
| Right End-PolyT 5-4 | TTTTTTGTTGTTCCAGTTTGGGGGAATTAGAGCCAGCTTTTT | (SEQ ID |
| | | NO: 1141) |
| Right End-PolyT 5-5 | TTTTTTTCTGTCCAGACGACGGATAAGTCCTGAACAATTTTT | (SEQ ID |
| | | NO: 1142) |
| Right End-PolyT 5-6 | TTTTTCATGTCAATCATATGTGCAAATGGTCAATAACTTTTT | (SEQ ID |
| | | NO: 1143) |
| Right End-PolyT 5-7 | GAGACAAAGGCTATCAGGTCATTGCCTTTTTT | (SEQ ID |
| | | NO: 1144) |
| Right End-PolyT 5-8 | TTTTTCTGTTTAGCTATATTTAATTACCTTATGCGATTTTTT | (SEQ ID |
| | | NO: 1145) |
| Right End-PolyT 5-9 | GCCATTTAACAAGAGAATAGCGGGCGCCACGT | (SEQ ID |
| | | NO: 1146) |
| Right End-PolyT 5- | TTTTTACAAAATCGCGCAGAGGCGAATT | (SEQ ID |
| 10 | | NO: 1147) |
| Right End-PolyT 5- | TGGTTCTCCGTCATCAACATTAAATGTGAGCGATTTTT | (SEQ ID |
| 11 | | NO: 1148) |
| Right End-PolyT 5- | TTTTTATTGCGTAGATTTTCACTTTGAATACCAAGTTTTTTT | (SEQ ID |
| 12 | | NO: 1149) |
| Right End-PolyT 5- | TTTTTCCAGACGTTAGTAAATAGGAATTGCGAATAATTTTTT | (SEQ ID |
| 13 | | NO: 1150) |
| Right End-PolyT 5- | TTTTTTTTAAGAACTGGCTCAACAAAAGGTAAAGTAATTTTT | (SEQ ID |
| 14 | | NO: 1151) |
| Right End-PolyT 5- | TTTTTTTCATCGGCATTTTCGGCCTCCCTCAGAGCCGTTTTT | (SEQ ID |
| 15 | | NO: 1152) |
| Right End-PolyT 5- | TTTGACATTTCACCCCGGATGAACGGTAATCGTAAAACTAGTTTTT | (SEQ ID |
| 16 | | NO: 1153) |
| Right End-PolyT 5- | TACCCATTGTGTCATTTGCGAACGACAA | (SEQ ID |
| 17 | | NO: 1154) |
| Right End-PolyT 5- | TTTTTGTAACAACCCGTCGGATGTAGATGGGCGCATCTTTTT | (SEQ ID |
| 18 | | NO: 1155) |
| Right End-PolyT 5- | TTTTTGAAAAATAATATCCCAATGACAACAACCATCGTTTTT | (SEQ ID |
| 19 | | NO: 1156) |
| Right End-PolyT 5- | TTTTTATTAAGTTGGGTAACGTTGATATAAGTATAGCTTTTT | (SEQ ID |
| 20 | | NO: 1157) |
| Right End-PolyT 5- | TTAATGAATCGAAACCTGATTAATTGCGTTGCGCTCACTGCTTTTT | (SEQ ID |
| 21 | | NO: 1158) |
| Right End-PolyT 5- | TTTTTCCGGAATAGGTGTATCAAGTTTTGTCGTCTTTTTTTT | (SEQ ID |
| 22 | | NO: 1159) |
| Right End-PolyT 5- | ATTGGGTTTAAGCACGTAATTAGAC | (SEQ ID |
| 23 | | NO: 1160) |
| Right End-PolyT 5- | TTTTTCCGCTTTCCAGTCGGGGCCAACGCGCGGGGAGTTTTT | (SEQ ID |
| 24 | | NO: 1161) |
| Right End-PolyT 5- | TTTTTAAAATCACCAGTAGCACAGACTGTAGCGCGTTTTTTT | (SEQ ID |
| 25 | | NO: 1162) |
| Right End-PolyT 5- | TTTTTTGGTCAGTTGGCAAATTCAATAGATAATACATTTTTT | (SEQ ID |
| 26 | | NO: 1163) |
| Right End-PolyT 5- | TTTTTTTGAGGATTTAGAAGTAAACAGAAATAAAGAATTTTT | (SEQ ID |
| 27 | | NO: 1164) |
| Right End-PolyT 5- | ATTCATTGATTCGCGTCGCTGTAGTTGCCAA | (SEQ ID |
| 28 | | NO: 1165) |
| Right End-PolyT 5- | TTTTTAATTTTTTCACGTTGAAAATCTCGCCGACATCCTAATAAC | (SEQ ID |
| 29 | | NO: 1166) |
| Right End-PolyT 5- | TTTTTGAGAGTCTGGAGCAAAACCACCAGCAGAAGATTTTTT | (SEQ ID |
| 30 | | NO: 1167) |
| Right End-PolyT 5- | TTTTCGATCTAACCGTACAGTACCGTTA | (SEQ ID |
| 31 | | NO: 1168) |
| Right End-PolyT 5- | TTTTTAAAACAGAGGTGAGGCACCCTCAATCAATATCTTTTT | (SEQ ID |
| 32 | | NO: 1169) |
| Right End-PolyT 5- | TTTTTCCACCCTCAGAACCGCCACCCTCCAACTAAGAA | (SEQ ID |
| 33 | | NO: 1170) |
| Left End-PolyT 5-1 | TTTTTTTAATTGCTGAATATTCACCAGTCACACGATTTTT | (SEQ ID |
| | | NO: 1171) |
| Left End-PolyT 5-2 | TTTTTTATGCGTTATACAAAACATAACGCCAAAAGTTTTT | (SEQ ID |
| | | NO: 1172) |
| Left End-PolyT 5-3 | TTTTTGAAGCCTTAAATCAAACCGTTCCAGTAAGCTTTTT | (SEQ ID |
| | | NO: 1173) |
| Left End-PolyT 5-4 | TTTTTGTCGGTGGGCACGAAATATTACCGCCAGCCTTTTT | (SEQ ID |
| | | NO: 1174) |
| Left End-PolyT 5-5 | TTTTTTAACCTTGCTTCTGTCGCCTGATAAATTGTTTTTT | (SEQ ID |
| | | NO: 1175) |
| Left End-PolyT 5-6 | AGAGAGAGGCTAATCATAAACCATGTTACTTAGCCGGAACTTTTT | (SEQ ID |
| | | NO: 1176) |
| Left End-PolyT 5-7 | CCGAAGCACAGAGATTTTTGTTTAACGTCAAAAATGTTTTT | (SEQ ID |
| | | NO: 1177) |
| Left End-PolyT 5-8 | TTTGATTAGTCTCCCGACTTGCGGGAGGTTTTTTTTT | (SEQ ID |
| | | NO: 1178) |
| Left End-PolyT 5-9 | GAACGGCCAACTTACATTTGG | (SEQ ID |
| | | NO: 1179) |
| Left End-PolyT 5-10 | TTTTTGTCATACATGGCTTTGTAACAGTGCCCGTATTTTT | (SEQ ID |
| | | NO: 1180) |
| Left End-PolyT 5-11 | TAGCATTAATTAATTTTCCCTTAGAATTTTT | (SEQ ID |
| | | NO: 1181) |
| Left End-PolyT 5-12 | CTTGTATCATAAATCGTTAAATCAATATATGTGAGTGAATTTTT | (SEQ ID |
| | | NO: 1182) |
| Left End-PolyT 5-13 | TTTTTATTAAGAGGAAGCCCTATTTTAAATGCAATTTTTT | (SEQ ID |
| | | NO: 1183) |
| Left End-PolyT 5-14 | TTTTTGAGGCGCAGACGGTCTTTGCAAAAGAAGTTTTTTT | (SEQ ID |
| | | NO: 1184) |
| Left End-PolyT 5-15 | TTTTTAAAATAGCAGCCTTTCCTTITTAAGAAAAGTTTTT | (SEQ ID |
| | | NO: 1185) |
| Left End-PolyT 5-16 | TTTTTCTACCTTTTTAACCTGCCTGTTTAGTATCATTTTT | (SEQ ID |
| | | NO: 1186) |
| Left End-PolyT 5-17 | TTTTTTAAGCAGATAGCCGAATAAGTTTATTTTGTTTTTT | (SEQ ID |
| | | NO: 1187) |
| Left End-PolyT 5-18 | TTTAGGTTGGGAATTTAACAGGAGCAGTCTCTACCAGATATCTTA | (SEQ ID |
| | | NO: 1188) |
| Left End-PolyT 5-19 | TTTTTGAATTACGAGGCATACGGATGGCTTAGAGCTTTTT | (SEQ ID |
| | | NO: 1189) |
| Left End-PolyT 5-20 | TTTTTATTGCAACAGGAAAAACGCTCAGGCAGATAATGCTGATTTT | (SEQ ID |
| | TGGTA | NO: 1190) |
| Left End-PolyT 5-21 | GTGCCCCTGCCGCCTTGATGATGATTCAAAATCGA | (SEQ ID |
| | | NO: 1191) |
| Left End-PolyT 5-22 | TTTTTGCCTGAGTAATGTGTAGGTAAAGATTCAATTA | (SEQ ID |
| | | NO: 1192) |
| Left End-PolyT 5-23 | TGCCCAAAATATGCAGATTTCTTACAGAAAAACCGG | (SEQ ID |
| | | NO: 1193) |
| Left End-PolyT 5-24 | ACTCAACTAAACCCCGCAGTGTCCACGTGGCGGAACCC | (SEQ ID |
| | | NO: 1194) |
| Left End-PolyT 5-25 | TTTTTCCAGTAATAAAAGGGACATTCTCCTCATAGAAA | (SEQ ID |
| | | NO: 1195) |
| Left End-PolyT 5-26 | TAAAGGGATTCATACCACGGAACAAAGTTGAA | (SEQ ID |
| | | NO: 1196) |
| Left End-PolyT 5-27 | TTTTTCACCACACCCGCCGCTCTTTGATTAGTAATTTTTT | (SEQ ID |
| | | NO: 1197) |
| Left End-PolyT 5-28 | TTTTTAACATCACTTGCCTGAGTAGAACAGAACATATAGGGCCC | (SEQ ID |
| | | NO: 1198) |
| Left End-PolyT 5-29 | TTTTTTTGCCAGAGGGGGTAGATTGCATCAAAAAGTTTTT | (SEQ ID |
| | | NO: 1199) |
| Left End-PolyT 5-30 | TTTTTATGACAATGTCCCGCATCTGTAAGCAACTCTTTTT | (SEQ ID |
| | | NO: 1200) |
| Left End-PolyT 5-31 | TTTTTTAAACAGTTAATGCCTGAATTGTCAACCTTTTTTT | (SEQ ID |
| | | NO: 1201) |
| Left End-PolyT 5-32 | TTTTTCACAATCAATAGAAAAGCCCCCGATTTAGATTTTT | (SEQ ID |
| | | NO: 1202) |
| Left End-PolyT 5-33 | TTTTTTCCTTGAAAACATAGCATAGGTCTGAGAGATTTTT | (SEQ ID |
| | | NO: 1203) |
| Left End-PolyT 5-34 | GACCAAAGCGATAGTAAAATGTTTAATAGCGCAAC | (SEQ ID |
| | | NO: 1204) |
| Left End-PolyT 5-35 | TTTTTGCTTGACGGGGAAAGCACGCTGCGCGTAACTTTTT | (SEQ ID |
| | | NO: 1205) |
| Left End-PolyT 5-36 | TTTTTGTCGAAATCCGCGACCTGCTTTATCATCAGAGA | (SEQ ID |
| | | NO: 1206) |
| Barcode connection 1 | ACCCAAATAATCTTACCGGCTTATACACGACGCTCTTCCGATCT | (SEQ ID |
| | | NO: 1207) |
| Barcode connection 2 | TATTCAGCTCCTCGAATTTCCACAAGGCCACACACGACGCTCTTCC | (SEQ ID |
| | GATCT | NO: 1208) |
| Barcode connection 3 | CGCGCCTTGAATATAATAACGTCAATTACCTGAGCACACGACGCTC | (SEQ ID |
| | TTCCGATCT | NO: 1209) |
| Barcode connection 4 | GTGAGCTAAACGGCGGATTGTTGGCCTTACACGACGCTCTTCCGA | (SEQ ID |
| | TCT | NO: 1210) |

### Example 2

### Nucleic Acid Nanostructure Delivery Compositions

The materials and methods below describe an example in which a DNA origami (DNAO) nanostructure is a cuboid structure and the nucleic acid barcode constructs are attached to the DNAO via complementary base pairing of the barcodes with one of the oligonucleotide staples within the DNAO.

### Barcode Design

The barcodes used in this example comprise a unique portion comprising 8 to 10 nucleotides in the center of the polynucleotide, the unique portion further characterized by a hamming distance of at least 3 bases from any other barcodes to be pooled. Directly on the 3′ end of the barcode, 7 to 10 random bases are included for bioinformatic removal of PCR duplicates. This central sequence is flanked by universal primer annealing sites containing overhangs for the addition of index adapters during sequencing library preparation. The polynucleotide barcodes in this example were designed with a biotin functional group on the 5′ end.

(SEQ ID NO: 1004)
/5BiosG/A*G*A*CGTGTGCTCTTCCGATCTGAGGGTACTTNNNNNNN
NNNAGATCGGAAGAGCGTCG*T*G*T.

### Materials for Nucleic Acid Nanostructure Delivery Composition Synthesis (DNAO)

The DNA origami scaffold is a single stranded DNA (ssDNA) isolated from the M13 bacteriophage. The oligonucleotide staples are short single stranded DNA with sequences described in Table 2. The barcode is a single stranded DNA segment as described under barcode design above.

### Preparation of the DNA Barcoded DNAO Structures

A reaction mixture was prepared comprising the DNA scaffold, the oligonucleotide staples and magnesium together in TE buffer in a reaction vessel in the following amounts: 160 uL all oligonucleotide staples (described in Table 2) pooled at a total concentration of 500 nM, 80 uL scaffold (100 nM), 80 uL water, 40 uL of TE buffer (1.46 g EDTA, 3.03 g Tris Base, 1.46 g NaCl, 500 mL water), 40 uL of 200 mM MgCl₂. The reaction vessel was placed in a thermocycler with thermal ramp starting at ˜65° C. and descending to 24° C. over the course of ˜67 hours. The product was purified using a precipitation with a PEG purification protocol using a PEG solution made with the following recipe: 75 g PEG8000, 50 mL of the TE buffer described above, and 62.5 mL of 4 M NaCl, brought up to 500 mL with water, yielding a DNAO nanostructure in water. The concentration was measured via nanodrop and then the barcodes were added to the product at a molar ratio of 4:1 (polynucleotide barcodes to DNAO nanostructure). This mixture was incubated at 37° C. for 2 to 3 hours. The product was purified with another PEG purification process as described above, yielding a final product of DNA barcoded DNAO in water. Transmission electron microscopy images were captured on formvar/carbon coated nickel grids with a negative stain with 1% phosphotungstic acid (PTA) using an FEI Tecnai G2 Bio Twin TEM (see FIG. 1).

### In Vitro Transfection

HEK 293 cells were plated in a 48 well plate at 75,000 cells/well with 200 μL of complete growth media 24 hours prior to transfection. Complete growth media consisted of DMEM supplemented with 10% FBS and 1% Pen-Strep. Cells were dosed with DNAO at a final concentration of 10 nM, 5 nM and 2.5 nM DNAO with and without barcode in triplicate for a total of 16 wells. Three (3) wells were used for controls. After 16 hours the cells were trypsinized and replicate wells pooled together. DNA from the cells was extracted using a Qiagen DNA Extraction Kit. These cell extracts were used for PCR amplification.

### Barcode PCR Amplification

A Master Mix was created with: Kapa HiFi 2x Master mix, Reverse barcode primer, forward barcode primer, DMSO, and nuclease free water. Master Mix (15 μL) was loaded into each well. Each of the cell extracts was loaded (5 μL) in duplicate into each well of a 96 well plate. Nuclease free water (5 μL) was loaded into the designated NTC wells. Positive control (5 μL) was loaded into the designated positive control wells. The positive control comprised a solution of polymer nanoparticles in phosphate buffered saline consisting of dimethylaminoethylmethacrylate, polyacylate, and butyl methacrylate, chemically conjugated with the same barcode as that used to label the DNAO, as described here before. Each well was covered, either with a strip cap or adhesive seal, and centrifuged for approx. 1 min at 1,000×g. Amplification of the barcodes was conducted by incubating in a thermocycler under typical PCR conditions.

### Analysis of PCR Amplification Via Gel Electrophoresis

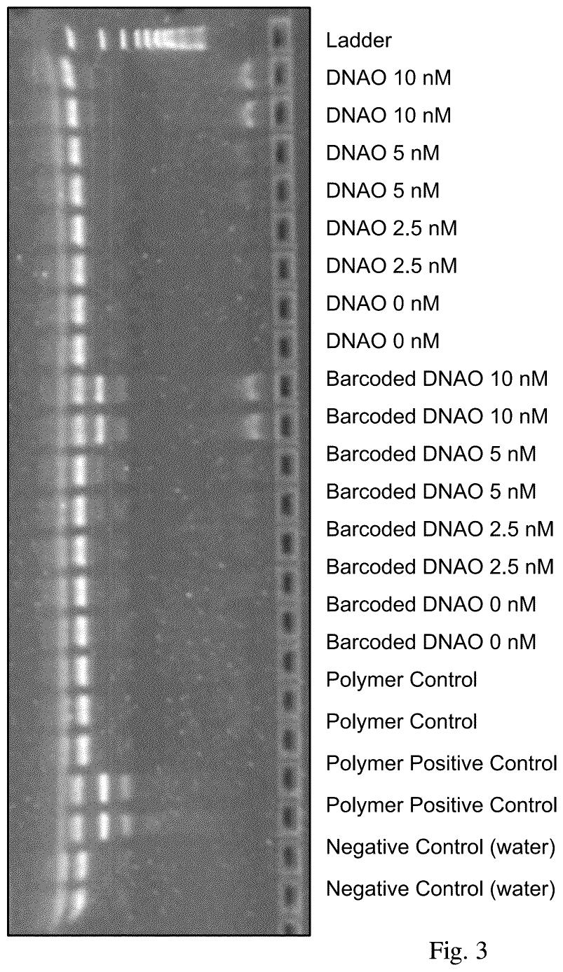

The gel electrophoresis was done on a 4% 48-well Ethidium Bromide gel, using 15 μL of 1 kb E-gel Ladder in the first well. DNAO and DNA barcoded DNAO cell extracts and controls (10 μL) were added to each well of the gel. E-gel buffer (5 μL) was added to each well. Negative controls from cells were extracts without any barcoded DNAO that underwent amplification. Negative controls in the last 2 wells were water. Nuclease free water (15 L) was added to any remaining empty wells. The gel doc was powered on to run current through the gel for about 20 to 25 mins, or until the sample buffer line reached the end of the gel. The gel was removed from the base and analyzed in a Gel Imager (see FIG. 3).

### Results

FIG. 3 shows PCR amplification of DNAO with and without barcodes at various transfection concentrations. Positive amplification is denoted by a distinct band above the primer bands (bright bands extending all the way across the gel towards the bottom of the gel) and a likeness to the positive control banding pattern. There is clear amplification of barcode from the cell extract of the 10 nM DNAO dose (two lanes above the primer bands) and some visible amplification of the 5 nM DNAO dose (two lanes above the primer bands). This confirms that the barcodes attached to the DNA origami structure were successfully delivered in the cells and can be read back at the right concentration. Note that the diffused bands near the wells at the top of the lane in the “DNAO 10 nM” and “Barcoded DNAO 10 nM” lanes are the actual DNA origami nanostructures.

1. A library of nucleic acid constructs comprising
a plurality of scaffold nucleic acids, each member of said plurality of scaffold nucleic acids being designed to fold into a different three-dimensional structure; and
a plurality of barcode sequences, said barcode sequences each comprising a first and second primer binding site that flank a detection sequence, wherein the detection sequence is different for each barcode sequence, further wherein each of the plurality of scaffold nucleic acids is linked to a different barcode sequence to form said nucleic acid constructs of the library.

2. The library of claim 1 further comprising a plurality of staple oligonucleotides that participate in folding the scaffold nucleic acids into said three-dimensional structure.

3. The library of claim 1 wherein the scaffold nucleic acid and the barcode sequence are comprised of DNA.

4. The library of claim 1 wherein each of the first binding sites are identical to one another, and each of the second binding sites are identical to one another, in each of said plurality of barcode sequences.

5. The library of claim 1 wherein each of the first binding sites are different from one another, and each of the second binding sites are different from one another, in each of said plurality of barcode sequences.

6. The library of claim 2, wherein the nucleic acid barcode constructs are linked to the scaffold nucleic acids or the staple oligonucleotides by a covalent bond.

7. The library of claim 2 wherein the nucleic acid barcode constructs are linked to the scaffold nucleic acids or the staple oligonucleotides via base-pair binding between complementary sequences of the respective scaffold nucleic acids or the staple oligonucleotides, and the nucleic acid barcode constructs.

8. The library of claim 1, wherein the nucleic acid barcode construct further comprises about 6 to about 12 random bases at the 3′ end of said barcode sequences.

9. The library of claim 1, further comprising a signaling molecule linked to said scaffold nucleic acid wherein the signaling molecule produces a signal detectable via imaging.

10. The library of claim 9 wherein the signaling molecule is a luminescent molecule.

11. A method of in vivo screening for a desired nucleic acid nanostructure delivery composition, the method comprising (a) preparing a library comprising two or more types of nucleic acid nanostructure delivery compositions, wherein each nucleic acid nanostructure delivery composition is associated with a nucleic acid barcode construct comprising a different barcode sequence, (b) administering the library to an animal, (c) removing cells or tissues from the animal, (d) isolating the nucleic acid barcode constructs from the cells or the tissues of the animal, (e) detecting the nucleic acid barcode constructs in the cells or the tissues of the animal, and (f) identifying the desired nucleic acid nanostructure delivery composition based on the detect location of the nucleic acid barcode constructs.

12. The method of claim 11, wherein the nucleic acid nanostructure delivery composition is loaded with a payload.

13. The method of claim 12, wherein the payload is a luminescent molecule.

14. The method of claim 13, wherein the luminescence is used to track the biodistribution or cell uptake of the nucleic acid nanostructure delivery composition via imaging.


SEQUENCE LISTING (1210 sequences)

SEQ ID NO: 1 - DNA, 10 bp, synthetic construct
  source 1..10: mol_type = other DNA
gctacataat

SEQ ID NO: 2 - DNA, 10 bp, synthetic construct
  source 1..10: mol_type = other DNA
atgttacaca

SEQ ID NO: 3 - DNA, 10 bp, synthetic construct
  source 1..10: mol_type = other DNA
tggggcccaa

SEQ ID NO: 4 - DNA, 10 bp, synthetic construct
  source 1..10: mol_type = other DNA
tagtttatcc

SEQ ID NO: 5 - DNA, 10 bp, synthetic construct
  source 1..10: mol_type = other DNA
accccgtctt

SEQ ID NO: 6 - DNA, 10 bp, synthetic construct
  source 1..10: mol_type = other DNA
ccggccatca

SEQ ID NO: 7 - DNA, 10 bp, synthetic construct
  source 1..10: mol_type = other DNA
gagcttgctc

SEQ ID NO: 8 - DNA, 10 bp, synthetic construct
  source 1..10: mol_type = other DNA
acgttctata

SEQ ID NO: 9 - DNA, 10 bp, synthetic construct
  source 1..10: mol_type = other DNA
tacagcaaaa

SEQ ID NO: 10 - DNA, 10 bp, synthetic construct
  source 1..10: mol_type = other DNA
gttaggtggt

SEQ ID NO: 11 - DNA, 10 bp, synthetic construct
  source 1..10: mol_type = other DNA
ggagaccgac

SEQ ID NO: 12 - DNA, 10 bp, synthetic construct
  source 1..10: mol_type = other DNA
tggccccttg

SEQ ID NO: 13 - DNA, 10 bp, synthetic construct
  source 1..10: mol_type = other DNA
tggccgtaag

SEQ ID NO: 14 - DNA, 10 bp, synthetic construct
  source 1..10: mol_type = other DNA
cgttcgtcaa

SEQ ID NO: 15 - DNA, 10 bp, synthetic construct
  source 1..10: mol_type = other DNA
cggacgtgga

SEQ ID NO: 16 - DNA, 10 bp, synthetic construct
  source 1..10: mol_type = other DNA
agagggggca

SEQ ID NO: 17 - DNA, 10 bp, synthetic construct
  source 1..10: mol_type = other DNA
gttcaggtcg

SEQ ID NO: 18 - DNA, 10 bp, synthetic construct
  source 1..10: mol_type = other DNA
ctcgcaagag

SEQ ID NO: 19 - DNA, 10 bp, synthetic construct
  source 1..10: mol_type = other DNA
gcaacgactt

SEQ ID NO: 20 - DNA, 10 bp, synthetic construct
  source 1..10: mol_type = other DNA
gccatccatc

SEQ ID NO: 21 - DNA, 10 bp, synthetic construct
  source 1..10: mol_type = other DNA
ttccgagcag

SEQ ID NO: 22 - DNA, 10 bp, synthetic construct
  source 1..10: mol_type = other DNA
cttctggaca

SEQ ID NO: 23 - DNA, 10 bp, synthetic construct
  source 1..10: mol_type = other DNA
aacattagac

SEQ ID NO: 24 - DNA, 10 bp, synthetic construct
  source 1..10: mol_type = other DNA
aagcaatagt

SEQ ID NO: 25 - DNA, 10 bp, synthetic construct
  source 1..10: mol_type = other DNA
agggtaagac

SEQ ID NO: 26 - DNA, 10 bp, synthetic construct
  source 1..10: mol_type = other DNA
cgttgtcttg

SEQ ID NO: 27 - DNA, 10 bp, synthetic construct
  source 1..10: mol_type = other DNA
tttccccgcc

SEQ ID NO: 28 - DNA, 10 bp, synthetic construct
  source 1..10: mol_type = other DNA
cgaatggatc

SEQ ID NO: 29 - DNA, 10 bp, synthetic construct
  source 1..10: mol_type = other DNA
catcacttgc

SEQ ID NO: 30 - DNA, 10 bp, synthetic construct
  source 1..10: mol_type = other DNA
ctctcgcact

SEQ ID NO: 31 - DNA, 10 bp, synthetic construct
  source 1..10: mol_type = other DNA
gttcacgtgc

SEQ ID NO: 32 - DNA, 10 bp, synthetic construct
  source 1..10: mol_type = other DNA
aataagcctg

SEQ ID NO: 33 - DNA, 10 bp, synthetic construct
  source 1..10: mol_type = other DNA
gttaacaatt

SEQ ID NO: 34 - DNA, 10 bp, synthetic construct
  source 1..10: mol_type = other DNA
attcagatcc

SEQ ID NO: 35 - DNA, 10 bp, synthetic construct
  source 1..10: mol_type = other DNA
cctgctgatt

SEQ ID NO: 36 - DNA, 10 bp, synthetic construct
  source 1..10: mol_type = other DNA
cttggtcata

SEQ ID NO: 37 - DNA, 10 bp, synthetic construct
  source 1..10: mol_type = other DNA
tcttcctgtt

SEQ ID NO: 38 - DNA, 10 bp, synthetic construct
  source 1..10: mol_type = other DNA
actgccatgg

SEQ ID NO: 39 - DNA, 10 bp, synthetic construct
  source 1..10: mol_type = other DNA
catgtatagt

SEQ ID NO: 40 - DNA, 10 bp, synthetic construct
  source 1..10: mol_type = other DNA
ggtagcggca

SEQ ID NO: 41 - DNA, 10 bp, synthetic construct
  source 1..10: mol_type = other DNA
tcactctaac

SEQ ID NO: 42 - DNA, 10 bp, synthetic construct
  source 1..10: mol_type = other DNA
aaggtgcacc

SEQ ID NO: 43 - DNA, 10 bp, synthetic construct
  source 1..10: mol_type = other DNA
aatgctcgtt

SEQ ID NO: 44 - DNA, 10 bp, synthetic construct
  source 1..10: mol_type = other DNA
tgtctagaaa

SEQ ID NO: 45 - DNA, 10 bp, synthetic construct
  source 1..10: mol_type = other DNA
ctgcctgcct

SEQ ID NO: 46 - DNA, 10 bp, synthetic construct
  source 1..10: mol_type = other DNA
actataaaag

SEQ ID NO: 47 - DNA, 10 bp, synthetic construct
  source 1..10: mol_type = other DNA
tagtatcgag

SEQ ID NO: 48 - DNA, 10 bp, synthetic construct
  source 1..10: mol_type = other DNA
atcgcagtcc

SEQ ID NO: 49 - DNA, 10 bp, synthetic construct
  source 1..10: mol_type = other DNA
tcatcagaac

SEQ ID NO: 50 - DNA, 10 bp, synthetic construct
  source 1..10: mol_type = other DNA
tcctagacgc

SEQ ID NO: 51 - DNA, 10 bp, synthetic construct
  source 1..10: mol_type = other DNA
gccgggcggg

SEQ ID NO: 52 - DNA, 10 bp, synthetic construct
  source 1..10: mol_type = other DNA
gcccagaaga

SEQ ID NO: 53 - DNA, 10 bp, synthetic construct
  source 1..10: mol_type = other DNA
cttagagctg

SEQ ID NO: 54 - DNA, 10 bp, synthetic construct
  source 1..10: mol_type = other DNA
gtctgcgctt

SEQ ID NO: 55 - DNA, 10 bp, synthetic construct
  source 1..10: mol_type = other DNA
cgccgtcctt

SEQ ID NO: 56 - DNA, 10 bp, synthetic construct
  source 1..10: mol_type = other DNA
tttatctgct

SEQ ID NO: 57 - DNA, 10 bp, synthetic construct
  source 1..10: mol_type = other DNA
tgcttcggag

SEQ ID NO: 58 - DNA, 10 bp, synthetic construct
  source 1..10: mol_type = other DNA
ggggagaatg

SEQ ID NO: 59 - DNA, 10 bp, synthetic construct
  source 1..10: mol_type = other DNA
gtggtaagtg

SEQ ID NO: 60 - DNA, 10 bp, synthetic construct
  source 1..10: mol_type = other DNA
gaaattagta

SEQ ID NO: 61 - DNA, 10 bp, synthetic construct
  source 1..10: mol_type = other DNA
gctatcctaa

SEQ ID NO: 62 - DNA, 10 bp, synthetic construct
  source 1..10: mol_type = other DNA
atctgtacga

SEQ ID NO: 63 - DNA, 10 bp, synthetic construct
  source 1..10: mol_type = other DNA
agttcggggc

SEQ ID NO: 64 - DNA, 10 bp, synthetic construct
  source 1..10: mol_type = other DNA
cgagtctgtc

SEQ ID NO: 65 - DNA, 10 bp, synthetic construct
  source 1..10: mol_type = other DNA
atcctacgca

SEQ ID NO: 66 - DNA, 10 bp, synthetic construct
  source 1..10: mol_type = other DNA
atggtggata

SEQ ID NO: 67 - DNA, 10 bp, synthetic construct
  source 1..10: mol_type = other DNA
cctctaacta

SEQ ID NO: 68 - DNA, 10 bp, synthetic construct
  source 1..10: mol_type = other DNA
atagctgcac

SEQ ID NO: 69 - DNA, 10 bp, synthetic construct
  source 1..10: mol_type = other DNA
gacagaattt

SEQ ID NO: 70 - DNA, 10 bp, synthetic construct
  source 1..10: mol_type = other DNA
caattggcat

SEQ ID NO: 71 - DNA, 10 bp, synthetic construct
  source 1..10: mol_type = other DNA
tctagtagac

SEQ ID NO: 72 - DNA, 10 bp, synthetic construct
  source 1..10: mol_type = other DNA
ttattcatgg

SEQ ID NO: 73 - DNA, 10 bp, synthetic construct
  source 1..10: mol_type = other DNA
ttggcaaccg

SEQ ID NO: 74 - DNA, 10 bp, synthetic construct
  source 1..10: mol_type = other DNA
cataatacat

SEQ ID NO: 75 - DNA, 10 bp, synthetic construct
  source 1..10: mol_type = other DNA
acagactcac

SEQ ID NO: 76 - DNA, 10 bp, synthetic construct
  source 1..10: mol_type = other DNA
gcgatgctgc

SEQ ID NO: 77 - DNA, 10 bp, synthetic construct
  source 1..10: mol_type = other DNA
catctttgcc

SEQ ID NO: 78 - DNA, 10 bp, synthetic construct
  source 1..10: mol_type = other DNA
gtgactccag

SEQ ID NO: 79 - DNA, 10 bp, synthetic construct
  source 1..10: mol_type = other DNA
ggacgagtct

SEQ ID NO: 80 - DNA, 10 bp, synthetic construct
  source 1..10: mol_type = other DNA
tagtggcgtg

SEQ ID NO: 81 - DNA, 10 bp, synthetic construct
  source 1..10: mol_type = other DNA
aacgcagctt

SEQ ID NO: 82 - DNA, 10 bp, synthetic construct
  source 1..10: mol_type = other DNA
agaacaggtg

SEQ ID NO: 83 - DNA, 10 bp, synthetic construct
  source 1..10: mol_type = other DNA
aggctatgtt

SEQ ID NO: 84 - DNA, 10 bp, synthetic construct
  source 1..10: mol_type = other DNA
cctggatctt

SEQ ID NO: 85 - DNA, 10 bp, synthetic construct
  source 1..10: mol_type = other DNA
ctagccggcc

SEQ ID NO: 86 - DNA, 10 bp, synthetic construct
  source 1..10: mol_type = other DNA
accagttatc

SEQ ID NO: 87 - DNA, 10 bp, synthetic construct
  source 1..10: mol_type = other DNA
acgttatagc

SEQ ID NO: 88 - DNA, 10 bp, synthetic construct
  source 1..10: mol_type = other DNA
tcgagtttga

SEQ ID NO: 89 - DNA, 10 bp, synthetic construct
  source 1..10: mol_type = other DNA
tgaagcgagc

SEQ ID NO: 90 - DNA, 10 bp, synthetic construct
  source 1..10: mol_type = other DNA
gactggcgaa

SEQ ID NO: 91 - DNA, 10 bp, synthetic construct
  source 1..10: mol_type = other DNA
gatggaccta

SEQ ID NO: 92 - DNA, 10 bp, synthetic construct
  source 1..10: mol_type = other DNA
gtccacaacg

SEQ ID NO: 93 - DNA, 10 bp, synthetic construct
  source 1..10: mol_type = other DNA
cctccccaga

SEQ ID NO: 94 - DNA, 10 bp, synthetic construct
  source 1..10: mol_type = other DNA
ttatgacgcc

SEQ ID NO: 95 - DNA, 10 bp, synthetic construct
  source 1..10: mol_type = other DNA
cttgatccgt

SEQ ID NO: 96 - DNA, 10 bp, synthetic construct
  source 1..10: mol_type = other DNA
aatgcgcaat

SEQ ID NO: 97 - DNA, 10 bp, synthetic construct
  source 1..10: mol_type = other DNA
gtacccctca

SEQ ID NO: 98 - DNA, 10 bp, synthetic construct
  source 1..10: mol_type = other DNA
cgacagctcg

SEQ ID NO: 99 - DNA, 10 bp, synthetic construct
  source 1..10: mol_type = other DNA
tgacctggct

SEQ ID NO: 100 - DNA, 10 bp, synthetic construct
  source 1..10: mol_type = other DNA
ttcatagccc

SEQ ID NO: 101 - DNA, 10 bp, synthetic construct
  source 1..10: mol_type = other DNA
cccaagagaa

SEQ ID NO: 102 - DNA, 10 bp, synthetic construct
  source 1..10: mol_type = other DNA
aaacgaagta

SEQ ID NO: 103 - DNA, 10 bp, synthetic construct
  source 1..10: mol_type = other DNA
gacgtttaca

SEQ ID NO: 104 - DNA, 10 bp, synthetic construct
  source 1..10: mol_type = other DNA
gatcgatttg

SEQ ID NO: 105 - DNA, 10 bp, synthetic construct
  source 1..10: mol_type = other DNA
cactgtcacc

SEQ ID NO: 106 - DNA, 10 bp, synthetic construct
  source 1..10: mol_type = other DNA
tgtgagagtt

SEQ ID NO: 107 - DNA, 10 bp, synthetic construct
  source 1..10: mol_type = other DNA
gacgtaacct

SEQ ID NO: 108 - DNA, 10 bp, synthetic construct
  source 1..10: mol_type = other DNA
cagactctgc

SEQ ID NO: 109 - DNA, 10 bp, synthetic construct
  source 1..10: mol_type = other DNA
tatgccaata

SEQ ID NO: 110 - DNA, 10 bp, synthetic construct
  source 1..10: mol_type = other DNA
acaggtgatg

SEQ ID NO: 111 - DNA, 10 bp, synthetic construct
  source 1..10: mol_type = other DNA
gtcatcgcgt

SEQ ID NO: 112 - DNA, 10 bp, synthetic construct
  source 1..10: mol_type = other DNA
tcttataaac

SEQ ID NO: 113 - DNA, 10 bp, synthetic construct
  source 1..10: mol_type = other DNA
gtgtagactg

SEQ ID NO: 114 - DNA, 10 bp, synthetic construct
  source 1..10: mol_type = other DNA
aaacaaccgg

SEQ ID NO: 115 - DNA, 10 bp, synthetic construct
  source 1..10: mol_type = other DNA
atcctgtacc

SEQ ID NO: 116 - DNA, 10 bp, synthetic construct
  source 1..10: mol_type = other DNA
ttataagaat

SEQ ID NO: 117 - DNA, 10 bp, synthetic construct
  source 1..10: mol_type = other DNA
ataagtaggc

SEQ ID NO: 118 - DNA, 10 bp, synthetic construct
  source 1..10: mol_type = other DNA
tctcgtaagg

SEQ ID NO: 119 - DNA, 10 bp, synthetic construct
  source 1..10: mol_type = other DNA
gatccgccgc

SEQ ID NO: 120 - DNA, 10 bp, synthetic construct
  source 1..10: mol_type = other DNA
tgtcaggttt

SEQ ID NO: 121 - DNA, 10 bp, synthetic construct
  source 1..10: mol_type = other DNA
tccgaagccc

SEQ ID NO: 122 - DNA, 10 bp, synthetic construct
  source 1..10: mol_type = other DNA
tccatgtcca

SEQ ID NO: 123 - DNA, 10 bp, synthetic construct
  source 1..10: mol_type = other DNA
gtgatggtac

SEQ ID NO: 124 - DNA, 10 bp, synthetic construct
  source 1..10: mol_type = other DNA
ctccacatac

SEQ ID NO: 125 - DNA, 10 bp, synthetic construct
  source 1..10: mol_type = other DNA
ttcggatgag

SEQ ID NO: 126 - DNA, 10 bp, synthetic construct
  source 1..10: mol_type = other DNA
acgacatcgc

SEQ ID NO: 127 - DNA, 10 bp, synthetic construct
  source 1..10: mol_type = other DNA
gagatgcaca

SEQ ID NO: 128 - DNA, 10 bp, synthetic construct
  source 1..10: mol_type = other DNA
tttgtatggc

SEQ ID NO: 129 - DNA, 10 bp, synthetic construct
  source 1..10: mol_type = other DNA
cttttctaga

SEQ ID NO: 130 - DNA, 10 bp, synthetic construct
  source 1..10: mol_type = other DNA
agtctaatca

SEQ ID NO: 131 - DNA, 10 bp, synthetic construct
  source 1..10: mol_type = other DNA
gacttagcca

SEQ ID NO: 132 - DNA, 10 bp, synthetic construct
  source 1..10: mol_type = other DNA
tatcacagta

SEQ ID NO: 133 - DNA, 10 bp, synthetic construct
  source 1..10: mol_type = other DNA
aagctcgagt

SEQ ID NO: 134 - DNA, 10 bp, synthetic construct
  source 1..10: mol_type = other DNA
tgttacgaca

SEQ ID NO: 135 - DNA, 10 bp, synthetic construct
  source 1..10: mol_type = other DNA
aaggatagtc

SEQ ID NO: 136 - DNA, 10 bp, synthetic construct
  source 1..10: mol_type = other DNA
gcacttagcc

SEQ ID NO: 137 - DNA, 10 bp, synthetic construct
  source 1..10: mol_type = other DNA
gagggatccg

SEQ ID NO: 138 - DNA, 10 bp, synthetic construct
  source 1..10: mol_type = other DNA
attctagaag

SEQ ID NO: 139 - DNA, 10 bp, synthetic construct
  source 1..10: mol_type = other DNA
gataactgat

SEQ ID NO: 140 - DNA, 10 bp, synthetic construct
  source 1..10: mol_type = other DNA
atctgactgt

SEQ ID NO: 141 - DNA, 10 bp, synthetic construct
  source 1..10: mol_type = other DNA
caaagcgaac

SEQ ID NO: 142 - DNA, 10 bp, synthetic construct
  source 1..10: mol_type = other DNA
gaaattgcga

SEQ ID NO: 143 - DNA, 10 bp, synthetic construct
  source 1..10: mol_type = other DNA
gggtccagtc

SEQ ID NO: 144 - DNA, 10 bp, synthetic construct
  source 1..10: mol_type = other DNA
atcaggtagc

SEQ ID NO: 145 - DNA, 10 bp, synthetic construct
  source 1..10: mol_type = other DNA
gaaaggtcct

SEQ ID NO: 146 - DNA, 10 bp, synthetic construct
  source 1..10: mol_type = other DNA
ggctaccaca

SEQ ID NO: 147 - DNA, 10 bp, synthetic construct
  source 1..10: mol_type = other DNA
ttattgctga

SEQ ID NO: 148 - DNA, 10 bp, synthetic construct
  source 1..10: mol_type = other DNA
cgccgcgttt

SEQ ID NO: 149 - DNA, 10 bp, synthetic construct
  source 1..10: mol_type = other DNA
ttttcaaaag

SEQ ID NO: 150 - DNA, 10 bp, synthetic construct
  source 1..10: mol_type = other DNA
ctgggctaaa

SEQ ID NO: 151 - DNA, 10 bp, synthetic construct
  source 1..10: mol_type = other DNA
cccgatgaga

SEQ ID NO: 152 - DNA, 10 bp, synthetic construct
  source 1..10: mol_type = other DNA
tgggaaatat

SEQ ID NO: 153 - DNA, 10 bp, synthetic construct
  source 1..10: mol_type = other DNA
gtacgagcgg

SEQ ID NO: 154 - DNA, 10 bp, synthetic construct
  source 1..10: mol_type = other DNA
gcgtgcagct

SEQ ID NO: 155 - DNA, 10 bp, synthetic construct
  source 1..10: mol_type = other DNA
agtctgcgga

SEQ ID NO: 156 - DNA, 10 bp, synthetic construct
  source 1..10: mol_type = other DNA
taactattta

SEQ ID NO: 157 - DNA, 10 bp, synthetic construct
  source 1..10: mol_type = other DNA
gagttgccgg

SEQ ID NO: 158 - DNA, 10 bp, synthetic construct
  source 1..10: mol_type = other DNA
cagcccggcg

SEQ ID NO: 159 - DNA, 10 bp, synthetic construct
  source 1..10: mol_type = other DNA
tcacctacat

SEQ ID NO: 160 - DNA, 10 bp, synthetic construct
  source 1..10: mol_type = other DNA
agtggctaac

SEQ ID NO: 161 - DNA, 10 bp, synthetic construct
  source 1..10: mol_type = other DNA
agaatgtgag

SEQ ID NO: 162 - DNA, 10 bp, synthetic construct
  source 1..10: mol_type = other DNA
tagtttcgca

SEQ ID NO: 163 - DNA, 10 bp, synthetic construct
  source 1..10: mol_type = other DNA
cttcatttct

SEQ ID NO: 164 - DNA, 10 bp, synthetic construct
  source 1..10: mol_type = other DNA
gccatgatat

SEQ ID NO: 165 - DNA, 10 bp, synthetic construct
  source 1..10: mol_type = other DNA
acggcaaatc

SEQ ID NO: 166 - DNA, 10 bp, synthetic construct
  source 1..10: mol_type = other DNA
atcgatagta

SEQ ID NO: 167 - DNA, 10 bp, synthetic construct
  source 1..10: mol_type = other DNA
cctaaaggca

SEQ ID NO: 168 - DNA, 10 bp, synthetic construct
  source 1..10: mol_type = other DNA
tacgagcggt

SEQ ID NO: 169 - DNA, 10 bp, synthetic construct
  source 1..10: mol_type = other DNA
tttgtcgtcg

SEQ ID NO: 170 - DNA, 10 bp, synthetic construct
  source 1..10: mol_type = other DNA
tacaagcttg

SEQ ID NO: 171 - DNA, 10 bp, synthetic construct
  source 1..10: mol_type = other DNA
gaccaacacg

SEQ ID NO: 172 - DNA, 10 bp, synthetic construct
  source 1..10: mol_type = other DNA
gaacgacgaa

SEQ ID NO: 173 - DNA, 10 bp, synthetic construct
  source 1..10: mol_type = other DNA
tcggaacgca

SEQ ID NO: 174 - DNA, 10 bp, synthetic construct
  source 1..10: mol_type = other DNA
atccggtggt

SEQ ID NO: 175 - DNA, 10 bp, synthetic construct
  source 1..10: mol_type = other DNA
taaaacgtag

SEQ ID NO: 176 - DNA, 10 bp, synthetic construct
  source 1..10: mol_type = other DNA
tatgtgagcc

SEQ ID NO: 177 - DNA, 10 bp, synthetic construct
  source 1..10: mol_type = other DNA
gaggcatcga

SEQ ID NO: 178 - DNA, 10 bp, synthetic construct
  source 1..10: mol_type = other DNA
gaatgggtgg

SEQ ID NO: 179 - DNA, 10 bp, synthetic construct
  source 1..10: mol_type = other DNA
aacgacacaa

SEQ ID NO: 180 - DNA, 10 bp, synthetic construct
  source 1..10: mol_type = other DNA
gtacgatgca

SEQ ID NO: 181 - DNA, 10 bp, synthetic construct
  source 1..10: mol_type = other DNA
agaaggcgcc

SEQ ID NO: 182 - DNA, 10 bp, synthetic construct
  source 1..10: mol_type = other DNA
ccgcaatgga

SEQ ID NO: 183 - DNA, 10 bp, synthetic construct
  source 1..10: mol_type = other DNA
tacggatttt

SEQ ID NO: 184 - DNA, 10 bp, synthetic construct
  source 1..10: mol_type = other DNA
gtcgttagct

SEQ ID NO: 185 - DNA, 10 bp, synthetic construct
  source 1..10: mol_type = other DNA
ggactagggc

SEQ ID NO: 186 - DNA, 10 bp, synthetic construct
  source 1..10: mol_type = other DNA
attggtattc

SEQ ID NO: 187 - DNA, 10 bp, synthetic construct
  source 1..10: mol_type = other DNA
atcccagaga

SEQ ID NO: 188 - DNA, 10 bp, synthetic construct
  source 1..10: mol_type = other DNA
gtcccagctc

SEQ ID NO: 189 - DNA, 10 bp, synthetic construct
  source 1..10: mol_type = other DNA
cacgaggaat

SEQ ID NO: 190 - DNA, 10 bp, synthetic construct
  source 1..10: mol_type = other DNA
tacaattgca

SEQ ID NO: 191 - DNA, 10 bp, synthetic construct
  source 1..10: mol_type = other DNA
attcctgaat

SEQ ID NO: 192 - DNA, 10 bp, synthetic construct
  source 1..10: mol_type = other DNA
tagcgaggcg

SEQ ID NO: 193 - DNA, 10 bp, synthetic construct
  source 1..10: mol_type = other DNA
ctggatgggc

SEQ ID NO: 194 - DNA, 10 bp, synthetic construct
  source 1..10: mol_type = other DNA
gcgacggcca

SEQ ID NO: 195 - DNA, 10 bp, synthetic construct
  source 1..10: mol_type = other DNA
acctgcacaa

SEQ ID NO: 196 - DNA, 10 bp, synthetic construct
  source 1..10: mol_type = other DNA
catgacagac

SEQ ID NO: 197 - DNA, 10 bp, synthetic construct
  source 1..10: mol_type = other DNA
ttaccaacgt

SEQ ID NO: 198 - DNA, 10 bp, synthetic construct
  source 1..10: mol_type = other DNA
caggtgtgtg

SEQ ID NO: 199 - DNA, 10 bp, synthetic construct
  source 1..10: mol_type = other DNA
cgagggacgg

SEQ ID NO: 200 - DNA, 10 bp, synthetic construct
  source 1..10: mol_type = other DNA
cgtctcggta

SEQ ID NO: 201 - DNA, 10 bp, synthetic construct
  source 1..10: mol_type = other DNA
taagctatct

SEQ ID NO: 202 - DNA, 10 bp, synthetic construct
  source 1..10: mol_type = other DNA
tactccccta

SEQ ID NO: 203 - DNA, 10 bp, synthetic construct
  source 1..10: mol_type = other DNA
ttatattcat

SEQ ID NO: 204 - DNA, 10 bp, synthetic construct
  source 1..10: mol_type = other DNA
agcgatctgc

SEQ ID NO: 205 - DNA, 10 bp, synthetic construct
  source 1..10: mol_type = other DNA
tcttctgatc

SEQ ID NO: 206 - DNA, 10 bp, synthetic construct
  source 1..10: mol_type = other DNA
atagttccca

SEQ ID NO: 207 - DNA, 10 bp, synthetic construct
  source 1..10: mol_type = other DNA
tttacgggtg

SEQ ID NO: 208 - DNA, 10 bp, synthetic construct
  source 1..10: mol_type = other DNA
gtgtcccctg

SEQ ID NO: 209 - DNA, 10 bp, synthetic construct
  source 1..10: mol_type = other DNA
gcgggggtcg

SEQ ID NO: 210 - DNA, 10 bp, synthetic construct
  source 1..10: mol_type = other DNA
cattgatcta

SEQ ID NO: 211 - DNA, 10 bp, synthetic construct
  source 1..10: mol_type = other DNA
agggacggtg

SEQ ID NO: 212 - DNA, 10 bp, synthetic construct
  source 1..10: mol_type = other DNA
cagttacttt

SEQ ID NO: 213 - DNA, 10 bp, synthetic construct
  source 1..10: mol_type = other DNA
ccatacttcc

SEQ ID NO: 214 - DNA, 10 bp, synthetic construct
  source 1..10: mol_type = other DNA
atcagaatta

SEQ ID NO: 215 - DNA, 10 bp, synthetic construct
  source 1..10: mol_type = other DNA
aaactaggca

SEQ ID NO: 216 - DNA, 10 bp, synthetic construct
  source 1..10: mol_type = other DNA
aatgtcgttg

SEQ ID NO: 217 - DNA, 10 bp, synthetic construct
  source 1..10: mol_type = other DNA
cacatgggtc

SEQ ID NO: 218 - DNA, 10 bp, synthetic construct
  source 1..10: mol_type = other DNA
ggtcgctggt

SEQ ID NO: 219 - DNA, 10 bp, synthetic construct
  source 1..10: mol_type = other DNA
actgtattac

SEQ ID NO: 220 - DNA, 10 bp, synthetic construct
  source 1..10: mol_type = other DNA
ccgagacgcg

SEQ ID NO: 221 - DNA, 10 bp, synthetic construct
  source 1..10: mol_type = other DNA
actccaaccc

SEQ ID NO: 222 - DNA, 10 bp, synthetic construct
  source 1..10: mol_type = other DNA
atattacaag

SEQ ID NO: 223 - DNA, 10 bp, synthetic construct
  source 1..10: mol_type = other DNA
ccatggatag

SEQ ID NO: 224 - DNA, 10 bp, synthetic construct
  source 1..10: mol_type = other DNA
ccgtctcaat

SEQ ID NO: 225 - DNA, 10 bp, synthetic construct
  source 1..10: mol_type = other DNA
gatcgtcggg

SEQ ID NO: 226 - DNA, 10 bp, synthetic construct
  source 1..10: mol_type = other DNA
tcttgttttg

SEQ ID NO: 227 - DNA, 10 bp, synthetic construct
  source 1..10: mol_type = other DNA
aatattgctc

SEQ ID NO: 228 - DNA, 10 bp, synthetic construct
  source 1..10: mol_type = other DNA
aacgtcgtct

SEQ ID NO: 229 - DNA, 10 bp, synthetic construct
  source 1..10: mol_type = other DNA
aatatttttg

SEQ ID NO: 230 - DNA, 10 bp, synthetic construct
  source 1..10: mol_type = other DNA
cgtaacgtgc

SEQ ID NO: 231 - DNA, 10 bp, synthetic construct
  source 1..10: mol_type = other DNA
gcgtggttat

SEQ ID NO: 232 - DNA, 10 bp, synthetic construct
  source 1..10: mol_type = other DNA
caaaacatta

SEQ ID NO: 233 - DNA, 10 bp, synthetic construct
  source 1..10: mol_type = other DNA
cgtatcctga

SEQ ID NO: 234 - DNA, 10 bp, synthetic construct
  source 1..10: mol_type = other DNA
tcgcttacaa

SEQ ID NO: 235 - DNA, 10 bp, synthetic construct
  source 1..10: mol_type = other DNA
tccattgtgt

SEQ ID NO: 236 - DNA, 10 bp, synthetic construct
  source 1..10: mol_type = other DNA
gcccccattc

SEQ ID NO: 237 - DNA, 10 bp, synthetic construct
  source 1..10: mol_type = other DNA
tgacgtctat

SEQ ID NO: 238 - DNA, 10 bp, synthetic construct
  source 1..10: mol_type = other DNA
tgggccgagg

SEQ ID NO: 239 - DNA, 10 bp, synthetic construct
  source 1..10: mol_type = other DNA
aagtgtcaag

SEQ ID NO: 240 - DNA, 10 bp, synthetic construct
  source 1..10: mol_type = other DNA
gacagtagag

SEQ ID NO: 241 - DNA, 10 bp, synthetic construct
  source 1..10: mol_type = other DNA
cgcagccatc

SEQ ID NO: 242 - DNA, 10 bp, synthetic construct
  source 1..10: mol_type = other DNA
gaggcagaac

SEQ ID NO: 243 - DNA, 10 bp, synthetic construct
  source 1..10: mol_type = other DNA
gttgaaattg

SEQ ID NO: 244 - DNA, 10 bp, synthetic construct
  source 1..10: mol_type = other DNA
atctgataaa

SEQ ID NO: 245 - DNA, 10 bp, synthetic construct
  source 1..10: mol_type = other DNA
agctgtctct

SEQ ID NO: 246 - DNA, 10 bp, synthetic construct
  source 1..10: mol_type = other DNA
ttttaggtta

SEQ ID NO: 247 - DNA, 10 bp, synthetic construct
  source 1..10: mol_type = other DNA
tatctgtccg

SEQ ID NO: 248 - DNA, 10 bp, synthetic construct
  source 1..10: mol_type = other DNA
aaaacatatg

SEQ ID NO: 249 - DNA, 10 bp, synthetic construct
  source 1..10: mol_type = other DNA
gtaaagaaga

SEQ ID NO: 250 - DNA, 10 bp, synthetic construct
  source 1..10: mol_type = other DNA
tcgacgtgca

SEQ ID NO: 251 - DNA, 10 bp, synthetic construct
  source 1..10: mol_type = other DNA
tagatcttaa

SEQ ID NO: 252 - DNA, 10 bp, synthetic construct
  source 1..10: mol_type = other DNA
cactggtcac

SEQ ID NO: 253 - DNA, 10 bp, synthetic construct
  source 1..10: mol_type = other DNA
attctgatgt

SEQ ID NO: 254 - DNA, 10 bp, synthetic construct
  source 1..10: mol_type = other DNA
atggccctga

SEQ ID NO: 255 - DNA, 10 bp, synthetic construct
  source 1..10: mol_type = other DNA
ggtgatgaga

SEQ ID NO: 256 - DNA, 10 bp, synthetic construct
  source 1..10: mol_type = other DNA
caccgtgggg

SEQ ID NO: 257 - DNA, 10 bp, synthetic construct
  source 1..10: mol_type = other DNA
gcttgctcgg

SEQ ID NO: 258 - DNA, 10 bp, synthetic construct
  source 1..10: mol_type = other DNA
ccagttgaac

SEQ ID NO: 259 - DNA, 10 bp, synthetic construct
  source 1..10: mol_type = other DNA
cgtctgtacc

SEQ ID NO: 260 - DNA, 10 bp, synthetic construct
  source 1..10: mol_type = other DNA
ccaacgcggc

SEQ ID NO: 261 - DNA, 10 bp, synthetic construct
  source 1..10: mol_type = other DNA
acgtgatcga

SEQ ID NO: 262 - DNA, 10 bp, synthetic construct
  source 1..10: mol_type = other DNA
ccatcgaatc

SEQ ID NO: 263 - DNA, 10 bp, synthetic construct
  source 1..10: mol_type = other DNA
cggtgtctgc

SEQ ID NO: 264 - DNA, 10 bp, synthetic construct
  source 1..10: mol_type = other DNA
aaaccacctc

SEQ ID NO: 265 - DNA, 10 bp, synthetic construct
  source 1..10: mol_type = other DNA
tcaatgttcc

SEQ ID NO: 266 - DNA, 10 bp, synthetic construct
  source 1..10: mol_type = other DNA
ttcgacatgt

SEQ ID NO: 267 - DNA, 10 bp, synthetic construct
  source 1..10: mol_type = other DNA
aggcacgata

SEQ ID NO: 268 - DNA, 10 bp, synthetic construct
  source 1..10: mol_type = other DNA
cacgagatca

SEQ ID NO: 269 - DNA, 10 bp, synthetic construct
  source 1..10: mol_type = other DNA
catgctgggg

SEQ ID NO: 270 - DNA, 10 bp, synthetic construct
  source 1..10: mol_type = other DNA
taccatggtt

SEQ ID NO: 271 - DNA, 10 bp, synthetic construct
  source 1..10: mol_type = other DNA
ttgcccatat

SEQ ID NO: 272 - DNA, 10 bp, synthetic construct
  source 1..10: mol_type = other DNA
tgcacattcg

SEQ ID NO: 273 - DNA, 10 bp, synthetic construct
  source 1..10: mol_type = other DNA
gttatgttgg

SEQ ID NO: 274 - DNA, 10 bp, synthetic construct
  source 1..10: mol_type = other DNA
tgagttatga

SEQ ID NO: 275 - DNA, 10 bp, synthetic construct
  source 1..10: mol_type = other DNA
gatggccccc

SEQ ID NO: 276 - DNA, 10 bp, synthetic construct
  source 1..10: mol_type = other DNA
gatgggttac

SEQ ID NO: 277 - DNA, 10 bp, synthetic construct
  source 1..10: mol_type = other DNA
agctacgttg

SEQ ID NO: 278 - DNA, 10 bp, synthetic construct
  source 1..10: mol_type = other DNA
accccatgca

SEQ ID NO: 279 - DNA, 10 bp, synthetic construct
  source 1..10: mol_type = other DNA
tactaccgtt

SEQ ID NO: 280 - DNA, 10 bp, synthetic construct
  source 1..10: mol_type = other DNA
tcgcttctac

SEQ ID NO: 281 - DNA, 10 bp, synthetic construct
  source 1..10: mol_type = other DNA
ctggcagtgc

SEQ ID NO: 282 - DNA, 10 bp, synthetic construct
  source 1..10: mol_type = other DNA
tctatatata

SEQ ID NO: 283 - DNA, 10 bp, synthetic construct
  source 1..10: mol_type = other DNA
ggattagttc

SEQ ID NO: 284 - DNA, 10 bp, synthetic construct
  source 1..10: mol_type = other DNA
gtgttacgct

SEQ ID NO: 285 - DNA, 10 bp, synthetic construct
  source 1..10: mol_type = other DNA
tcgactccgt

SEQ ID NO: 286 - DNA, 10 bp, synthetic construct
  source 1..10: mol_type = other DNA
ggtagcaggc

SEQ ID NO: 287 - DNA, 10 bp, synthetic construct
  source 1..10: mol_type = other DNA
tattggattc

SEQ ID NO: 288 - DNA, 10 bp, synthetic construct
  source 1..10: mol_type = other DNA
gttcgatcga

SEQ ID NO: 289 - DNA, 10 bp, synthetic construct
  source 1..10: mol_type = other DNA
atattaatat

SEQ ID NO: 290 - DNA, 10 bp, synthetic construct
  source 1..10: mol_type = other DNA
agaacgattg

SEQ ID NO: 291 - DNA, 10 bp, synthetic construct
  source 1..10: mol_type = other DNA
gtaaagtgta

SEQ ID NO: 292 - DNA, 10 bp, synthetic construct
  source 1..10: mol_type = other DNA
cccatgtgcc

SEQ ID NO: 293 - DNA, 10 bp, synthetic construct
  source 1..10: mol_type = other DNA
gtggcctcgc

SEQ ID NO: 294 - DNA, 10 bp, synthetic construct
  source 1..10: mol_type = other DNA
gacactagga

SEQ ID NO: 295 - DNA, 10 bp, synthetic construct
  source 1..10: mol_type = other DNA
atattctgac

SEQ ID NO: 296 - DNA, 10 bp, synthetic construct
  source 1..10: mol_type = other DNA
taagtagacg

SEQ ID NO: 297 - DNA, 10 bp, synthetic construct
  source 1..10: mol_type = other DNA
taacggtcta

SEQ ID NO: 298 - DNA, 10 bp, synthetic construct
  source 1..10: mol_type = other DNA
tagtttcatt

SEQ ID NO: 299 - DNA, 10 bp, synthetic construct
  source 1..10: mol_type = other DNA
ttggatccga

SEQ ID NO: 300 - DNA, 10 bp, synthetic construct
  source 1..10: mol_type = other DNA
cgtgacaacc

SEQ ID NO: 301 - DNA, 10 bp, synthetic construct
  source 1..10: mol_type = other DNA
cgcgctcaga

SEQ ID NO: 302 - DNA, 10 bp, synthetic construct
  source 1..10: mol_type = other DNA
cgttcttaat

SEQ ID NO: 303 - DNA, 10 bp, synthetic construct
  source 1..10: mol_type = other DNA
acaagagttt

SEQ ID NO: 304 - DNA, 10 bp, synthetic construct
  source 1..10: mol_type = other DNA
agggttatag

SEQ ID NO: 305 - DNA, 10 bp, synthetic construct
  source 1..10: mol_type = other DNA
accacgactc

SEQ ID NO: 306 - DNA, 10 bp, synthetic construct
  source 1..10: mol_type = other DNA
gtactcgggg

SEQ ID NO: 307 - DNA, 10 bp, synthetic construct
  source 1..10: mol_type = other DNA
acaaatatct

SEQ ID NO: 308 - DNA, 10 bp, synthetic construct
  source 1..10: mol_type = other DNA
gatcggggtg

SEQ ID NO: 309 - DNA, 10 bp, synthetic construct
  source 1..10: mol_type = other DNA
atgtaactcc

SEQ ID NO: 310 - DNA, 10 bp, synthetic construct
  source 1..10: mol_type = other DNA
atgaagaagc

SEQ ID NO: 311 - DNA, 10 bp, synthetic construct
  source 1..10: mol_type = other DNA
atgtattgtc

SEQ ID NO: 312 - DNA, 10 bp, synthetic construct
  source 1..10: mol_type = other DNA
tgcattggaa

SEQ ID NO: 313 - DNA, 10 bp, synthetic construct
  source 1..10: mol_type = other DNA
gcggacgatc

SEQ ID NO: 314 - DNA, 10 bp, synthetic construct
  source 1..10: mol_type = other DNA
ccgtacttga

SEQ ID NO: 315 - DNA, 10 bp, synthetic construct
  source 1..10: mol_type = other DNA
tttgcccccg

SEQ ID NO: 316 - DNA, 10 bp, synthetic construct
  source 1..10: mol_type = other DNA
acctcacgcg

SEQ ID NO: 317 - DNA, 10 bp, synthetic construct
  source 1..10: mol_type = other DNA
attaaggggc

SEQ ID NO: 318 - DNA, 10 bp, synthetic construct
  source 1..10: mol_type = other DNA
cgtggacatg

SEQ ID NO: 319 - DNA, 10 bp, synthetic construct
  source 1..10: mol_type = other DNA
ttagcccttc

SEQ ID NO: 320 - DNA, 10 bp, synthetic construct
  source 1..10: mol_type = other DNA
cgagagtttg

SEQ ID NO: 321 - DNA, 10 bp, synthetic construct
  source 1..10: mol_type = other DNA
tgcatcctct

SEQ ID NO: 322 - DNA, 10 bp, synthetic construct
  source 1..10: mol_type = other DNA
tgcgattccg

SEQ ID NO: 323 - DNA, 10 bp, synthetic construct
  source 1..10: mol_type = other DNA
ttattacgtt

SEQ ID NO: 324 - DNA, 10 bp, synthetic construct
  source 1..10: mol_type = other DNA
tgatgtggtt

SEQ ID NO: 325 - DNA, 10 bp, synthetic construct
  source 1..10: mol_type = other DNA
gggcgtcaat

SEQ ID NO: 326 - DNA, 10 bp, synthetic construct
  source 1..10: mol_type = other DNA
cccttgaaat

SEQ ID NO: 327 - DNA, 10 bp, synthetic construct
  source 1..10: mol_type = other DNA
tctttggggc

SEQ ID NO: 328 - DNA, 10 bp, synthetic construct
  source 1..10: mol_type = other DNA
accggcaggc

SEQ ID NO: 329 - DNA, 10 bp, synthetic construct
  source 1..10: mol_type = other DNA
gctaaaatct

SEQ ID NO: 330 - DNA, 10 bp, synthetic construct
  source 1..10: mol_type = other DNA
gccgttgacg

SEQ ID NO: 331 - DNA, 10 bp, synthetic construct
  source 1..10: mol_type = other DNA
ggagttgttg

SEQ ID NO: 332 - DNA, 10 bp, synthetic construct
  source 1..10: mol_type = other DNA
tacttgagaa

SEQ ID NO: 333 - DNA, 10 bp, synthetic construct
  source 1..10: mol_type = other DNA
cgggtgcgct

SEQ ID NO: 334 - DNA, 10 bp, synthetic construct
  source 1..10: mol_type = other DNA
aaaagcgtct

SEQ ID NO: 335 - DNA, 10 bp, synthetic construct
  source 1..10: mol_type = other DNA
gtaaagatag

SEQ ID NO: 336 - DNA, 10 bp, synthetic construct
  source 1..10: mol_type = other DNA
gcctggtcag

SEQ ID NO: 337 - DNA, 10 bp, synthetic construct
  source 1..10: mol_type = other DNA
ggcaaaaagg

SEQ ID NO: 338 - DNA, 10 bp, synthetic construct
  source 1..10: mol_type = other DNA
acccttctct

SEQ ID NO: 339 - DNA, 10 bp, synthetic construct
  source 1..10: mol_type = other DNA
tcacatagtg

SEQ ID NO: 340 - DNA, 10 bp, synthetic construct
  source 1..10: mol_type = other DNA
tcgtctgtgc

SEQ ID NO: 341 - DNA, 10 bp, synthetic construct
  source 1..10: mol_type = other DNA
tgctcggatc

SEQ ID NO: 342 - DNA, 10 bp, synthetic construct
  source 1..10: mol_type = other DNA
agcagtcccg

SEQ ID NO: 343 - DNA, 10 bp, synthetic construct
  source 1..10: mol_type = other DNA
tttgggctgt

SEQ ID NO: 344 - DNA, 10 bp, synthetic construct
  source 1..10: mol_type = other DNA
ctcacgatct

SEQ ID NO: 345 - DNA, 10 bp, synthetic construct
  source 1..10: mol_type = other DNA
tggcgcatac

SEQ ID NO: 346 - DNA, 10 bp, synthetic construct
  source 1..10: mol_type = other DNA
gcaattgaaa

SEQ ID NO: 347 - DNA, 10 bp, synthetic construct
  source 1..10: mol_type = other DNA
tcgggagacg

SEQ ID NO: 348 - DNA, 10 bp, synthetic construct
  source 1..10: mol_type = other DNA
cccggcgaaa

SEQ ID NO: 349 - DNA, 10 bp, synthetic construct
  source 1..10: mol_type = other DNA
tgatgcggaa

SEQ ID NO: 350 - DNA, 10 bp, synthetic construct
  source 1..10: mol_type = other DNA
aactgaggcg

SEQ ID NO: 351 - DNA, 10 bp, synthetic construct
  source 1..10: mol_type = other DNA
catattattt

SEQ ID NO: 352 - DNA, 10 bp, synthetic construct
  source 1..10: mol_type = other DNA
aaaagtcatt

SEQ ID NO: 353 - DNA, 10 bp, synthetic construct
  source 1..10: mol_type = other DNA
aagcggtgag

SEQ ID NO: 354 - DNA, 10 bp, synthetic construct
  source 1..10: mol_type = other DNA
aaggtaatca

SEQ ID NO: 355 - DNA, 10 bp, synthetic construct
  source 1..10: mol_type = other DNA
ctgacactta

SEQ ID NO: 356 - DNA, 10 bp, synthetic construct
  source 1..10: mol_type = other DNA
ctgttttcta

SEQ ID NO: 357 - DNA, 10 bp, synthetic construct
  source 1..10: mol_type = other DNA
cacatggcag

SEQ ID NO: 358 - DNA, 10 bp, synthetic construct
  source 1..10: mol_type = other DNA
ttcaatccgg

SEQ ID NO: 359 - DNA, 10 bp, synthetic construct
  source 1..10: mol_type = other DNA
tgtccggcat

SEQ ID NO: 360 - DNA, 10 bp, synthetic construct
  source 1..10: mol_type = other DNA
tggtaccgtg

SEQ ID NO: 361 - DNA, 10 bp, synthetic construct
  source 1..10: mol_type = other DNA
aagagatatt

SEQ ID NO: 362 - DNA, 10 bp, synthetic construct
  source 1..10: mol_type = other DNA
gatgtactac

SEQ ID NO: 363 - DNA, 10 bp, synthetic construct
  source 1..10: mol_type = other DNA
gaaatggaat

SEQ ID NO: 364 - DNA, 10 bp, synthetic construct
  source 1..10: mol_type = other DNA
ttaaaatact

SEQ ID NO: 365 - DNA, 10 bp, synthetic construct
  source 1..10: mol_type = other DNA
tgaccggaac

SEQ ID NO: 366 - DNA, 10 bp, synthetic construct
  source 1..10: mol_type = other DNA
gtcgccgcaa

SEQ ID NO: 367 - DNA, 10 bp, synthetic construct
  source 1..10: mol_type = other DNA
taggataccg

SEQ ID NO: 368 - DNA, 10 bp, synthetic construct
  source 1..10: mol_type = other DNA
agtccaattg

SEQ ID NO: 369 - DNA, 10 bp, synthetic construct
  source 1..10: mol_type = other DNA
gggggctata

SEQ ID NO: 370 - DNA, 10 bp, synthetic construct
  source 1..10: mol_type = other DNA
accttcagtt

SEQ ID NO: 371 - DNA, 10 bp, synthetic construct
  source 1..10: mol_type = other DNA
atggcaagta

SEQ ID NO: 372 - DNA, 10 bp, synthetic construct
  source 1..10: mol_type = other DNA
agaatgtttt

SEQ ID NO: 373 - DNA, 10 bp, synthetic construct
  source 1..10: mol_type = other DNA
agttcgtttg

SEQ ID NO: 374 - DNA, 10 bp, synthetic construct
  source 1..10: mol_type = other DNA
cactactgac

SEQ ID NO: 375 - DNA, 10 bp, synthetic construct
  source 1..10: mol_type = other DNA
gatcaagagc

SEQ ID NO: 376 - DNA, 10 bp, synthetic construct
  source 1..10: mol_type = other DNA
atttatcgag

SEQ ID NO: 377 - DNA, 10 bp, synthetic construct
  source 1..10: mol_type = other DNA
cctttttcca

SEQ ID NO: 378 - DNA, 10 bp, synthetic construct
  source 1..10: mol_type = other DNA
gcacagaggt

SEQ ID NO: 379 - DNA, 10 bp, synthetic construct
  source 1..10: mol_type = other DNA
tgatctgaat

SEQ ID NO: 380 - DNA, 10 bp, synthetic construct
  source 1..10: mol_type = other DNA
gttggaggga

SEQ ID NO: 381 - DNA, 10 bp, synthetic construct
  source 1..10: mol_type = other DNA
ttttgaaggt

SEQ ID NO: 382 - DNA, 10 bp, synthetic construct
  source 1..10: mol_type = other DNA
taagtcctaa

SEQ ID NO: 383 - DNA, 10 bp, synthetic construct
  source 1..10: mol_type = other DNA
ggtgttaggg

SEQ ID NO: 384 - DNA, 10 bp, synthetic construct
  source 1..10: mol_type = other DNA
tgtatgcacc

SEQ ID NO: 385 - DNA, 10 bp, synthetic construct
  source 1..10: mol_type = other DNA
ccgtgccatt

SEQ ID NO: 386 - DNA, 10 bp, synthetic construct
  source 1..10: mol_type = other DNA
gaaatcaccc

SEQ ID NO: 387 - DNA, 10 bp, synthetic construct
  source 1..10: mol_type = other DNA
tttgcacgtg

SEQ ID NO: 388 - DNA, 10 bp, synthetic construct
  source 1..10: mol_type = other DNA
cgtctgtttt

SEQ ID NO: 389 - DNA, 10 bp, synthetic construct
  source 1..10: mol_type = other DNA
ctacaccaca

SEQ ID NO: 390 - DNA, 10 bp, synthetic construct
  source 1..10: mol_type = other DNA
tgctacaggg

SEQ ID NO: 391 - DNA, 10 bp, synthetic construct
  source 1..10: mol_type = other DNA
gggaatatat

SEQ ID NO: 392 - DNA, 10 bp, synthetic construct
  source 1..10: mol_type = other DNA
tcatgtattt

SEQ ID NO: 393 - DNA, 10 bp, synthetic construct
  source 1..10: mol_type = other DNA
tctccgttta

SEQ ID NO: 394 - DNA, 10 bp, synthetic construct
  source 1..10: mol_type = other DNA
tacctctcgc

SEQ ID NO: 395 - DNA, 10 bp, synthetic construct
  source 1..10: mol_type = other DNA
gcttcaaccg

SEQ ID NO: 396 - DNA, 10 bp, synthetic construct
  source 1..10: mol_type = other DNA
atgaagctac

SEQ ID NO: 397 - DNA, 10 bp, synthetic construct
  source 1..10: mol_type = other DNA
cggtacaact

SEQ ID NO: 398 - DNA, 10 bp, synthetic construct
  source 1..10: mol_type = other DNA
gtgtggtcgt

SEQ ID NO: 399 - DNA, 10 bp, synthetic construct
  source 1..10: mol_type = other DNA
ggggtcatgt

SEQ ID NO: 400 - DNA, 10 bp, synthetic construct
  source 1..10: mol_type = other DNA
aggcagccca

SEQ ID NO: 401 - DNA, 10 bp, synthetic construct
  source 1..10: mol_type = other DNA
caagcacgat

SEQ ID NO: 402 - DNA, 10 bp, synthetic construct
  source 1..10: mol_type = other DNA
tcaaatggat

SEQ ID NO: 403 - DNA, 10 bp, synthetic construct
  source 1..10: mol_type = other DNA
ggactgaata

SEQ ID NO: 404 - DNA, 10 bp, synthetic construct
  source 1..10: mol_type = other DNA
ccgtagacgt

SEQ ID NO: 405 - DNA, 10 bp, synthetic construct
  source 1..10: mol_type = other DNA
cggcgtaccg

SEQ ID NO: 406 - DNA, 10 bp, synthetic construct
  source 1..10: mol_type = other DNA
ggcggcgccc

SEQ ID NO: 407 - DNA, 10 bp, synthetic construct
  source 1..10: mol_type = other DNA
agacttgatc

SEQ ID NO: 408 - DNA, 10 bp, synthetic construct
  source 1..10: mol_type = other DNA
accttgcaca

SEQ ID NO: 409 - DNA, 10 bp, synthetic construct
  source 1..10: mol_type = other DNA
taaggtgagt

SEQ ID NO: 410 - DNA, 10 bp, synthetic construct
  source 1..10: mol_type = other DNA
ttgttgtttc

SEQ ID NO: 411 - DNA, 10 bp, synthetic construct
  source 1..10: mol_type = other DNA
gagggaatac

SEQ ID NO: 412 - DNA, 10 bp, synthetic construct
  source 1..10: mol_type = other DNA
ctcgtacgcg

SEQ ID NO: 413 - DNA, 10 bp, synthetic construct
  source 1..10: mol_type = other DNA
ccgcggttta

SEQ ID NO: 414 - DNA, 10 bp, synthetic construct
  source 1..10: mol_type = other DNA
ttaaagttaa

SEQ ID NO: 415 - DNA, 10 bp, synthetic construct
  source 1..10: mol_type = other DNA
gcatatgggt

SEQ ID NO: 416 - DNA, 10 bp, synthetic construct
  source 1..10: mol_type = other DNA
agtctgagcc

SEQ ID NO: 417 - DNA, 10 bp, synthetic construct
  source 1..10: mol_type = other DNA
tgtcggttcg

SEQ ID NO: 418 - DNA, 10 bp, synthetic construct
  source 1..10: mol_type = other DNA
ggtctcaacc

SEQ ID NO: 419 - DNA, 10 bp, synthetic construct
  source 1..10: mol_type = other DNA
gtaacggcat

SEQ ID NO: 420 - DNA, 10 bp, synthetic construct
  source 1..10: mol_type = other DNA
acactgagaa

SEQ ID NO: 421 - DNA, 10 bp, synthetic construct
  source 1..10: mol_type = other DNA
cccaacgtcg

SEQ ID NO: 422 - DNA, 10 bp, synthetic construct
  source 1..10: mol_type = other DNA
aagaaactgc

SEQ ID NO: 423 - DNA, 10 bp, synthetic construct
  source 1..10: mol_type = other DNA
accagcccac

SEQ ID NO: 424 - DNA, 10 bp, synthetic construct
  source 1..10: mol_type = other DNA
tgtagttact

SEQ ID NO: 425 - DNA, 10 bp, synthetic construct
  source 1..10: mol_type = other DNA
ggctagaggc

SEQ ID NO: 426 - DNA, 10 bp, synthetic construct
  source 1..10: mol_type = other DNA
gttcggcaga

SEQ ID NO: 427 - DNA, 10 bp, synthetic construct
  source 1..10: mol_type = other DNA
ccaaaataga

SEQ ID NO: 428 - DNA, 10 bp, synthetic construct
  source 1..10: mol_type = other DNA
cccatataac

SEQ ID NO: 429 - DNA, 10 bp, synthetic construct
  source 1..10: mol_type = other DNA
gtcactaccg

SEQ ID NO: 430 - DNA, 10 bp, synthetic construct
  source 1..10: mol_type = other DNA
gtagtgtggc

SEQ ID NO: 431 - DNA, 10 bp, synthetic construct
  source 1..10: mol_type = other DNA
caatctcata

SEQ ID NO: 432 - DNA, 10 bp, synthetic construct
  source 1..10: mol_type = other DNA
ccatgttata

SEQ ID NO: 433 - DNA, 10 bp, synthetic construct
  source 1..10: mol_type = other DNA
taagcagtgg

SEQ ID NO: 434 - DNA, 10 bp, synthetic construct
  source 1..10: mol_type = other DNA
tcggcggcta

SEQ ID NO: 435 - DNA, 10 bp, synthetic construct
  source 1..10: mol_type = other DNA
tattaaatgc

SEQ ID NO: 436 - DNA, 10 bp, synthetic construct
  source 1..10: mol_type = other DNA
gtcgccatta

SEQ ID NO: 437 - DNA, 10 bp, synthetic construct
  source 1..10: mol_type = other DNA
ggcgtcgttc

SEQ ID NO: 438 - DNA, 10 bp, synthetic construct
  source 1..10: mol_type = other DNA
ctagtagata

SEQ ID NO: 439 - DNA, 10 bp, synthetic construct
  source 1..10: mol_type = other DNA
tcgtcagtat

SEQ ID NO: 440 - DNA, 10 bp, synthetic construct
  source 1..10: mol_type = other DNA
ggggtatcgg

SEQ ID NO: 441 - DNA, 10 bp, synthetic construct
  source 1..10: mol_type = other DNA
tgctctgcca

SEQ ID NO: 442 - DNA, 10 bp, synthetic construct
  source 1..10: mol_type = other DNA
tgccgtaact

SEQ ID NO: 443 - DNA, 10 bp, synthetic construct
  source 1..10: mol_type = other DNA
cggtacaggc

SEQ ID NO: 444 - DNA, 10 bp, synthetic construct
  source 1..10: mol_type = other DNA
tcctaatttg

SEQ ID NO: 445 - DNA, 10 bp, synthetic construct
  source 1..10: mol_type = other DNA
tctttctgga

SEQ ID NO: 446 - DNA, 10 bp, synthetic construct
  source 1..10: mol_type = other DNA
ccgcgacttg

SEQ ID NO: 447 - DNA, 10 bp, synthetic construct
  source 1..10: mol_type = other DNA
acctatagcg

SEQ ID NO: 448 - DNA, 10 bp, synthetic construct
  source 1..10: mol_type = other DNA
gccggcacct

SEQ ID NO: 449 - DNA, 10 bp, synthetic construct
  source 1..10: mol_type = other DNA
tttgataggc

SEQ ID NO: 450 - DNA, 10 bp, synthetic construct
  source 1..10: mol_type = other DNA
actgtgagct

SEQ ID NO: 451 - DNA, 10 bp, synthetic construct
  source 1..10: mol_type = other DNA
ttatcgttca

SEQ ID NO: 452 - DNA, 10 bp, synthetic construct
  source 1..10: mol_type = other DNA
actagtggcc

SEQ ID NO: 453 - DNA, 10 bp, synthetic construct
  source 1..10: mol_type = other DNA
cctccgtggt

SEQ ID NO: 454 - DNA, 10 bp, synthetic construct
  source 1..10: mol_type = other DNA
ttagggtatg

SEQ ID NO: 455 - DNA, 10 bp, synthetic construct
  source 1..10: mol_type = other DNA
gaatcaggcg

SEQ ID NO: 456 - DNA, 10 bp, synthetic construct
  source 1..10: mol_type = other DNA
ggctgaccaa

SEQ ID NO: 457 - DNA, 10 bp, synthetic construct
  source 1..10: mol_type = other DNA
tgccagaccg

SEQ ID NO: 458 - DNA, 10 bp, synthetic construct
  source 1..10: mol_type = other DNA
tccctacgcg

SEQ ID NO: 459 - DNA, 10 bp, synthetic construct
  source 1..10: mol_type = other DNA
tccgctggag

SEQ ID NO: 460 - DNA, 10 bp, synthetic construct
  source 1..10: mol_type = other DNA
ggatcaaaac

SEQ ID NO: 461 - DNA, 10 bp, synthetic construct
  source 1..10: mol_type = other DNA
ttcacctcac

SEQ ID NO: 462 - DNA, 10 bp, synthetic construct
  source 1..10: mol_type = other DNA
gacacacggc

SEQ ID NO: 463 - DNA, 10 bp, synthetic construct
  source 1..10: mol_type = other DNA
tgggcgatta

SEQ ID NO: 464 - DNA, 10 bp, synthetic construct
  source 1..10: mol_type = other DNA
taagatcttc

SEQ ID NO: 465 - DNA, 10 bp, synthetic construct
  source 1..10: mol_type = other DNA
ctccgactac

SEQ ID NO: 466 - DNA, 10 bp, synthetic construct
  source 1..10: mol_type = other DNA
gggccatcat

SEQ ID NO: 467 - DNA, 10 bp, synthetic construct
  source 1..10: mol_type = other DNA
tcaggccaga

SEQ ID NO: 468 - DNA, 10 bp, synthetic construct
  source 1..10: mol_type = other DNA
cttgtggggc

SEQ ID NO: 469 - DNA, 10 bp, synthetic construct
  source 1..10: mol_type = other DNA
agatagtctg

SEQ ID NO: 470 - DNA, 10 bp, synthetic construct
  source 1..10: mol_type = other DNA
gcgtcaaagt

SEQ ID NO: 471 - DNA, 10 bp, synthetic construct
  source 1..10: mol_type = other DNA
acgaaaattt

SEQ ID NO: 472 - DNA, 10 bp, synthetic construct
  source 1..10: mol_type = other DNA
gagtctggtg

SEQ ID NO: 473 - DNA, 10 bp, synthetic construct
  source 1..10: mol_type = other DNA
atcgagcgac

SEQ ID NO: 474 - DNA, 10 bp, synthetic construct
  source 1..10: mol_type = other DNA
ggtcctcaga

SEQ ID NO: 475 - DNA, 10 bp, synthetic construct
  source 1..10: mol_type = other DNA
tgattttgtc

SEQ ID NO: 476 - DNA, 10 bp, synthetic construct
  source 1..10: mol_type = other DNA
gcatttctca

SEQ ID NO: 477 - DNA, 10 bp, synthetic construct
  source 1..10: mol_type = other DNA
gcatgccagt

SEQ ID NO: 478 - DNA, 10 bp, synthetic construct
  source 1..10: mol_type = other DNA
attagacgac

SEQ ID NO: 479 - DNA, 10 bp, synthetic construct
  source 1..10: mol_type = other DNA
aaagcccata

SEQ ID NO: 480 - DNA, 10 bp, synthetic construct
  source 1..10: mol_type = other DNA
cactacattc

SEQ ID NO: 481 - DNA, 10 bp, synthetic construct
  source 1..10: mol_type = other DNA
cacggtttct

SEQ ID NO: 482 - DNA, 10 bp, synthetic construct
  source 1..10: mol_type = other DNA
cccaccagtg

SEQ ID NO: 483 - DNA, 10 bp, synthetic construct
  source 1..10: mol_type = other DNA
ctcacttgtc

SEQ ID NO: 484 - DNA, 10 bp, synthetic construct
  source 1..10: mol_type = other DNA
gatagactct

SEQ ID NO: 485 - DNA, 10 bp, synthetic construct
  source 1..10: mol_type = other DNA
atttccattt

SEQ ID NO: 486 - DNA, 10 bp, synthetic construct
  source 1..10: mol_type = other DNA
atatgtggcc

SEQ ID NO: 487 - DNA, 10 bp, synthetic construct
  source 1..10: mol_type = other DNA
cgggacgaac

SEQ ID NO: 488 - DNA, 10 bp, synthetic construct
  source 1..10: mol_type = other DNA
agaaccgtga

SEQ ID NO: 489 - DNA, 10 bp, synthetic construct
  source 1..10: mol_type = other DNA
tagtgtactg

SEQ ID NO: 490 - DNA, 10 bp, synthetic construct
  source 1..10: mol_type = other DNA
aactaatcga

SEQ ID NO: 491 - DNA, 10 bp, synthetic construct
  source 1..10: mol_type = other DNA
cgaagtgacg

SEQ ID NO: 492 - DNA, 10 bp, synthetic construct
  source 1..10: mol_type = other DNA
cggagcctcg

SEQ ID NO: 493 - DNA, 10 bp, synthetic construct
  source 1..10: mol_type = other DNA
atcacacgag

SEQ ID NO: 494 - DNA, 10 bp, synthetic construct
  source 1..10: mol_type = other DNA
cgacgagttc

SEQ ID NO: 495 - DNA, 10 bp, synthetic construct
  source 1..10: mol_type = other DNA
gcttcccgtg

SEQ ID NO: 496 - DNA, 10 bp, synthetic construct
  source 1..10: mol_type = other DNA
gattcatacc

SEQ ID NO: 497 - DNA, 10 bp, synthetic construct
  source 1..10: mol_type = other DNA
gagagaagcg

SEQ ID NO: 498 - DNA, 10 bp, synthetic construct
  source 1..10: mol_type = other DNA
gaagtggcct

SEQ ID NO: 499 - DNA, 10 bp, synthetic construct
  source 1..10: mol_type = other DNA
ggacgacgcc

SEQ ID NO: 500 - DNA, 10 bp, synthetic construct
  source 1..10: mol_type = other DNA
tagggtctca

SEQ ID NO: 501 - DNA, 10 bp, synthetic construct
  source 1..10: mol_type = other DNA
aactacaggt

SEQ ID NO: 502 - DNA, 10 bp, synthetic construct
  source 1..10: mol_type = other DNA
gtggcctgtg

SEQ ID NO: 503 - DNA, 10 bp, synthetic construct
  source 1..10: mol_type = other DNA
ctttaccagc

SEQ ID NO: 504 - DNA, 10 bp, synthetic construct
  source 1..10: mol_type = other DNA
cgcgttactg

SEQ ID NO: 505 - DNA, 10 bp, synthetic construct
  source 1..10: mol_type = other DNA
ttgctcccgt

SEQ ID NO: 506 - DNA, 10 bp, synthetic construct
  source 1..10: mol_type = other DNA
catcaaacaa

SEQ ID NO: 507 - DNA, 10 bp, synthetic construct
  source 1..10: mol_type = other DNA
gctttatgat

SEQ ID NO: 508 - DNA, 10 bp, synthetic construct
  source 1..10: mol_type = other DNA
ctgcatactg

SEQ ID NO: 509 - DNA, 10 bp, synthetic construct
  source 1..10: mol_type = other DNA
ggtggctcag

SEQ ID NO: 510 - DNA, 10 bp, synthetic construct
  source 1..10: mol_type = other DNA
ggacgatcaa

SEQ ID NO: 511 - DNA, 10 bp, synthetic construct
  source 1..10: mol_type = other DNA
ccgactggtg

SEQ ID NO: 512 - DNA, 10 bp, synthetic construct
  source 1..10: mol_type = other DNA
ggaacaaccg

SEQ ID NO: 513 - DNA, 10 bp, synthetic construct
  source 1..10: mol_type = other DNA
gaacgagacc

SEQ ID NO: 514 - DNA, 10 bp, synthetic construct
  source 1..10: mol_type = other DNA
caccaagaaa

SEQ ID NO: 515 - DNA, 10 bp, synthetic construct
  source 1..10: mol_type = other DNA
atgcattacc

SEQ ID NO: 516 - DNA, 10 bp, synthetic construct
  source 1..10: mol_type = other DNA
gtatcatgcc

SEQ ID NO: 517 - DNA, 10 bp, synthetic construct
  source 1..10: mol_type = other DNA
agtagatgtt

SEQ ID NO: 518 - DNA, 10 bp, synthetic construct
  source 1..10: mol_type = other DNA
ctctagatgt

SEQ ID NO: 519 - DNA, 10 bp, synthetic construct
  source 1..10: mol_type = other DNA
gctacttgtg

SEQ ID NO: 520 - DNA, 10 bp, synthetic construct
  source 1..10: mol_type = other DNA
tatgaaacgt

SEQ ID NO: 521 - DNA, 10 bp, synthetic construct
  source 1..10: mol_type = other DNA
cctcgttgat

SEQ ID NO: 522 - DNA, 10 bp, synthetic construct
  source 1..10: mol_type = other DNA
ctagagccat

SEQ ID NO: 523 - DNA, 10 bp, synthetic construct
  source 1..10: mol_type = other DNA
tagagttata

SEQ ID NO: 524 - DNA, 10 bp, synthetic construct
  source 1..10: mol_type = other DNA
aacgagaggc

SEQ ID NO: 525 - DNA, 10 bp, synthetic construct
  source 1..10: mol_type = other DNA
ggtctaccgt

SEQ ID NO: 526 - DNA, 10 bp, synthetic construct
  source 1..10: mol_type = other DNA
gccccctcac

SEQ ID NO: 527 - DNA, 10 bp, synthetic construct
  source 1..10: mol_type = other DNA
cataggaatt

SEQ ID NO: 528 - DNA, 10 bp, synthetic construct
  source 1..10: mol_type = other DNA
tccggctcgt

SEQ ID NO: 529 - DNA, 10 bp, synthetic construct
  source 1..10: mol_type = other DNA
tgagagtcgg

SEQ ID NO: 530 - DNA, 10 bp, synthetic construct
  source 1..10: mol_type = other DNA
cgtagaaata

SEQ ID NO: 531 - DNA, 10 bp, synthetic construct
  source 1..10: mol_type = other DNA
ctttacatga

SEQ ID NO: 532 - DNA, 10 bp, synthetic construct
  source 1..10: mol_type = other DNA
gagcgccgtc

SEQ ID NO: 533 - DNA, 10 bp, synthetic construct
  source 1..10: mol_type = other DNA
ggctctcggc

SEQ ID NO: 534 - DNA, 10 bp, synthetic construct
  source 1..10: mol_type = other DNA
agagcttgtt

SEQ ID NO: 535 - DNA, 10 bp, synthetic construct
  source 1..10: mol_type = other DNA
aatcagccac

SEQ ID NO: 536 - DNA, 10 bp, synthetic construct
  source 1..10: mol_type = other DNA
agaagagcca

SEQ ID NO: 537 - DNA, 10 bp, synthetic construct
  source 1..10: mol_type = other DNA
tcgtatgagt

SEQ ID NO: 538 - DNA, 10 bp, synthetic construct
  source 1..10: mol_type = other DNA
ttcttcctcg

SEQ ID NO: 539 - DNA, 10 bp, synthetic construct
  source 1..10: mol_type = other DNA
acacaaaagc

SEQ ID NO: 540 - DNA, 10 bp, synthetic construct
  source 1..10: mol_type = other DNA
cgcgggaccc

SEQ ID NO: 541 - DNA, 10 bp, synthetic construct
  source 1..10: mol_type = other DNA
gtcgcgacac

SEQ ID NO: 542 - DNA, 10 bp, synthetic construct
  source 1..10: mol_type = other DNA
ccggaggaaa

SEQ ID NO: 543 - DNA, 10 bp, synthetic construct
  source 1..10: mol_type = other DNA
cggcgtatga

SEQ ID NO: 544 - DNA, 10 bp, synthetic construct
  source 1..10: mol_type = other DNA
taggcattct

SEQ ID NO: 545 - DNA, 10 bp, synthetic construct
  source 1..10: mol_type = other DNA
aaaggaggga

SEQ ID NO: 546 - DNA, 10 bp, synthetic construct
  source 1..10: mol_type = other DNA
acctttacgg

SEQ ID NO: 547 - DNA, 10 bp, synthetic construct
  source 1..10: mol_type = other DNA
ctaccgttaa

SEQ ID NO: 548 - DNA, 10 bp, synthetic construct
  source 1..10: mol_type = other DNA
gagcttcgcc

SEQ ID NO: 549 - DNA, 10 bp, synthetic construct
  source 1..10: mol_type = other DNA
gccatagaag

SEQ ID NO: 550 - DNA, 10 bp, synthetic construct
  source 1..10: mol_type = other DNA
tttagcgtat

SEQ ID NO: 551 - DNA, 10 bp, synthetic construct
  source 1..10: mol_type = other DNA
gcaaacagat

SEQ ID NO: 552 - DNA, 10 bp, synthetic construct
  source 1..10: mol_type = other DNA
taggtcatgg

SEQ ID NO: 553 - DNA, 10 bp, synthetic construct
  source 1..10: mol_type = other DNA
ctctaacaga

SEQ ID NO: 554 - DNA, 10 bp, synthetic construct
  source 1..10: mol_type = other DNA
ggctcatgaa

SEQ ID NO: 555 - DNA, 10 bp, synthetic construct
  source 1..10: mol_type = other DNA
caatgtctca

SEQ ID NO: 556 - DNA, 10 bp, synthetic construct
  source 1..10: mol_type = other DNA
tgatcgtatt

SEQ ID NO: 557 - DNA, 10 bp, synthetic construct
  source 1..10: mol_type = other DNA
gcgcttttca

SEQ ID NO: 558 - DNA, 10 bp, synthetic construct
  source 1..10: mol_type = other DNA
aagattatat

SEQ ID NO: 559 - DNA, 10 bp, synthetic construct
  source 1..10: mol_type = other DNA
actagctgac

SEQ ID NO: 560 - DNA, 10 bp, synthetic construct
  source 1..10: mol_type = other DNA
ggtgagctca

SEQ ID NO: 561 - DNA, 10 bp, synthetic construct
  source 1..10: mol_type = other DNA
cgctttcgct

SEQ ID NO: 562 - DNA, 10 bp, synthetic construct
  source 1..10: mol_type = other DNA
tgattcaaaa

SEQ ID NO: 563 - DNA, 10 bp, synthetic construct
  source 1..10: mol_type = other DNA
actgaacagg

SEQ ID NO: 564 - DNA, 10 bp, synthetic construct
  source 1..10: mol_type = other DNA
attcgagcta

SEQ ID NO: 565 - DNA, 10 bp, synthetic construct
  source 1..10: mol_type = other DNA
tgtaggctaa

SEQ ID NO: 566 - DNA, 10 bp, synthetic construct
  source 1..10: mol_type = other DNA
acaaagcttt

SEQ ID NO: 567 - DNA, 10 bp, synthetic construct
  source 1..10: mol_type = other DNA
gcccgaggga

SEQ ID NO: 568 - DNA, 10 bp, synthetic construct
  source 1..10: mol_type = other DNA
gcccgctggg

SEQ ID NO: 569 - DNA, 10 bp, synthetic construct
  source 1..10: mol_type = other DNA
accccgctga

SEQ ID NO: 570 - DNA, 10 bp, synthetic construct
  source 1..10: mol_type = other DNA
cttatgccct

SEQ ID NO: 571 - DNA, 10 bp, synthetic construct
  source 1..10: mol_type = other DNA
ccgccatagc

SEQ ID NO: 572 - DNA, 10 bp, synthetic construct
  source 1..10: mol_type = other DNA
cttaatgatt

SEQ ID NO: 573 - DNA, 10 bp, synthetic construct
  source 1..10: mol_type = other DNA
cagtccacaa

SEQ ID NO: 574 - DNA, 10 bp, synthetic construct
  source 1..10: mol_type = other DNA
atggacggac

SEQ ID NO: 575 - DNA, 10 bp, synthetic construct
  source 1..10: mol_type = other DNA
cggcctctcg

SEQ ID NO: 576 - DNA, 10 bp, synthetic construct
  source 1..10: mol_type = other DNA
tagtcgccat

SEQ ID NO: 577 - DNA, 10 bp, synthetic construct
  source 1..10: mol_type = other DNA
gttgatcttc

SEQ ID NO: 578 - DNA, 10 bp, synthetic construct
  source 1..10: mol_type = other DNA
acttgccaag

SEQ ID NO: 579 - DNA, 10 bp, synthetic construct
  source 1..10: mol_type = other DNA
atgactggtt

SEQ ID NO: 580 - DNA, 10 bp, synthetic construct
  source 1..10: mol_type = other DNA
tgtcgtagga

SEQ ID NO: 581 - DNA, 10 bp, synthetic construct
  source 1..10: mol_type = other DNA
agcaaacacg

SEQ ID NO: 582 - DNA, 10 bp, synthetic construct
  source 1..10: mol_type = other DNA
tactgatgaa

SEQ ID NO: 583 - DNA, 10 bp, synthetic construct
  source 1..10: mol_type = other DNA
gtatcccata

SEQ ID NO: 584 - DNA, 10 bp, synthetic construct
  source 1..10: mol_type = other DNA
tagccaggtt

SEQ ID NO: 585 - DNA, 10 bp, synthetic construct
  source 1..10: mol_type = other DNA
cgtgtggcga

SEQ ID NO: 586 - DNA, 10 bp, synthetic construct
  source 1..10: mol_type = other DNA
atcgaattgc

SEQ ID NO: 587 - DNA, 10 bp, synthetic construct
  source 1..10: mol_type = other DNA
ccccaatatt

SEQ ID NO: 588 - DNA, 10 bp, synthetic construct
  source 1..10: mol_type = other DNA
cccgtttctc

SEQ ID NO: 589 - DNA, 10 bp, synthetic construct
  source 1..10: mol_type = other DNA
tccgcatcta

SEQ ID NO: 590 - DNA, 10 bp, synthetic construct
  source 1..10: mol_type = other DNA
caagcctcat

SEQ ID NO: 591 - DNA, 10 bp, synthetic construct
  source 1..10: mol_type = other DNA
tttcaatccc

SEQ ID NO: 592 - DNA, 10 bp, synthetic construct
  source 1..10: mol_type = other DNA
ccttcccatc

SEQ ID NO: 593 - DNA, 10 bp, synthetic construct
  source 1..10: mol_type = other DNA
aggtacaaga

SEQ ID NO: 594 - DNA, 10 bp, synthetic construct
  source 1..10: mol_type = other DNA
gtgtaatgga

SEQ ID NO: 595 - DNA, 10 bp, synthetic construct
  source 1..10: mol_type = other DNA
aaactgagct

SEQ ID NO: 596 - DNA, 10 bp, synthetic construct
  source 1..10: mol_type = other DNA
atctctgccc

SEQ ID NO: 597 - DNA, 10 bp, synthetic construct
  source 1..10: mol_type = other DNA
cgacatttgc

SEQ ID NO: 598 - DNA, 10 bp, synthetic construct
  source 1..10: mol_type = other DNA
tgtgaacccg

SEQ ID NO: 599 - DNA, 10 bp, synthetic construct
  source 1..10: mol_type = other DNA
tgacacccca

SEQ ID NO: 600 - DNA, 10 bp, synthetic construct
  source 1..10: mol_type = other DNA
taggccaaag

SEQ ID NO: 601 - DNA, 10 bp, synthetic construct
  source 1..10: mol_type = other DNA
gaaattgtag

SEQ ID NO: 602 - DNA, 10 bp, synthetic construct
  source 1..10: mol_type = other DNA
gcgtctgatt

SEQ ID NO: 603 - DNA, 10 bp, synthetic construct
  source 1..10: mol_type = other DNA
tctcattgtt

SEQ ID NO: 604 - DNA, 10 bp, synthetic construct
  source 1..10: mol_type = other DNA
ctgacatctc

SEQ ID NO: 605 - DNA, 10 bp, synthetic construct
  source 1..10: mol_type = other DNA
gtatccagtg

SEQ ID NO: 606 - DNA, 10 bp, synthetic construct
  source 1..10: mol_type = other DNA
gatggccgtt

SEQ ID NO: 607 - DNA, 10 bp, synthetic construct
  source 1..10: mol_type = other DNA
tcaccctctc

SEQ ID NO: 608 - DNA, 10 bp, synthetic construct
  source 1..10: mol_type = other DNA
ggcactattc

SEQ ID NO: 609 - DNA, 10 bp, synthetic construct
  source 1..10: mol_type = other DNA
aaataactgt

SEQ ID NO: 610 - DNA, 10 bp, synthetic construct
  source 1..10: mol_type = other DNA
cagctccatt

SEQ ID NO: 611 - DNA, 10 bp, synthetic construct
  source 1..10: mol_type = other DNA
ctcttgactc

SEQ ID NO: 612 - DNA, 10 bp, synthetic construct
  source 1..10: mol_type = other DNA
tttcctatac

SEQ ID NO: 613 - DNA, 10 bp, synthetic construct
  source 1..10: mol_type = other DNA
ccatacccga

SEQ ID NO: 614 - DNA, 10 bp, synthetic construct
  source 1..10: mol_type = other DNA
tcgccgagcg

SEQ ID NO: 615 - DNA, 10 bp, synthetic construct
  source 1..10: mol_type = other DNA
cgctgaagcc

SEQ ID NO: 616 - DNA, 10 bp, synthetic construct
  source 1..10: mol_type = other DNA
tctggcccca

SEQ ID NO: 617 - DNA, 10 bp, synthetic construct
  source 1..10: mol_type = other DNA
gctacattga

SEQ ID NO: 618 - DNA, 10 bp, synthetic construct
  source 1..10: mol_type = other DNA
cgcatcataa

SEQ ID NO: 619 - DNA, 10 bp, synthetic construct
  source 1..10: mol_type = other DNA
gcaaagggcc

SEQ ID NO: 620 - DNA, 10 bp, synthetic construct
  source 1..10: mol_type = other DNA
aacggcgcag

SEQ ID NO: 621 - DNA, 10 bp, synthetic construct
  source 1..10: mol_type = other DNA
cgactgacat

SEQ ID NO: 622 - DNA, 10 bp, synthetic construct
  source 1..10: mol_type = other DNA
atgacagggc

SEQ ID NO: 623 - DNA, 10 bp, synthetic construct
  source 1..10: mol_type = other DNA
caagttctcc

SEQ ID NO: 624 - DNA, 10 bp, synthetic construct
  source 1..10: mol_type = other DNA
tcgccgcttt

SEQ ID NO: 625 - DNA, 10 bp, synthetic construct
  source 1..10: mol_type = other DNA
atgccggaaa

SEQ ID NO: 626 - DNA, 10 bp, synthetic construct
  source 1..10: mol_type = other DNA
gcggttacta

SEQ ID NO: 627 - DNA, 10 bp, synthetic construct
  source 1..10: mol_type = other DNA
gacattacaa

SEQ ID NO: 628 - DNA, 10 bp, synthetic construct
  source 1..10: mol_type = other DNA
cagagagggc

SEQ ID NO: 629 - DNA, 10 bp, synthetic construct
  source 1..10: mol_type = other DNA
gcaccgcctc

SEQ ID NO: 630 - DNA, 10 bp, synthetic construct
  source 1..10: mol_type = other DNA
cggtccgagc

SEQ ID NO: 631 - DNA, 10 bp, synthetic construct
  source 1..10: mol_type = other DNA
tgtccggtgc

SEQ ID NO: 632 - DNA, 10 bp, synthetic construct
  source 1..10: mol_type = other DNA
ggtcggttgc

SEQ ID NO: 633 - DNA, 10 bp, synthetic construct
  source 1..10: mol_type = other DNA
gctcagctaa

SEQ ID NO: 634 - DNA, 10 bp, synthetic construct
  source 1..10: mol_type = other DNA
agcagttcgt

SEQ ID NO: 635 - DNA, 10 bp, synthetic construct
  source 1..10: mol_type = other DNA
aaatcgatga

SEQ ID NO: 636 - DNA, 10 bp, synthetic construct
  source 1..10: mol_type = other DNA
gctcggtatg

SEQ ID NO: 637 - DNA, 10 bp, synthetic construct
  source 1..10: mol_type = other DNA
cccgccgcgg

SEQ ID NO: 638 - DNA, 10 bp, synthetic construct
  source 1..10: mol_type = other DNA
gtgtgatagg

SEQ ID NO: 639 - DNA, 10 bp, synthetic construct
  source 1..10: mol_type = other DNA
ttggactcca

SEQ ID NO: 640 - DNA, 10 bp, synthetic construct
  source 1..10: mol_type = other DNA
tgcttatcta

SEQ ID NO: 641 - DNA, 10 bp, synthetic construct
  source 1..10: mol_type = other DNA
caaaaggcgt

SEQ ID NO: 642 - DNA, 10 bp, synthetic construct
  source 1..10: mol_type = other DNA
tagggggcct

SEQ ID NO: 643 - DNA, 10 bp, synthetic construct
  source 1..10: mol_type = other DNA
aagtattaat

SEQ ID NO: 644 - DNA, 10 bp, synthetic construct
  source 1..10: mol_type = other DNA
gtttagcccg

SEQ ID NO: 645 - DNA, 10 bp, synthetic construct
  source 1..10: mol_type = other DNA
cgctaatatg

SEQ ID NO: 646 - DNA, 10 bp, synthetic construct
  source 1..10: mol_type = other DNA
acaacacgtt

SEQ ID NO: 647 - DNA, 10 bp, synthetic construct
  source 1..10: mol_type = other DNA
agagatgctc

SEQ ID NO: 648 - DNA, 10 bp, synthetic construct
  source 1..10: mol_type = other DNA
tgcctgatat

SEQ ID NO: 649 - DNA, 10 bp, synthetic construct
  source 1..10: mol_type = other DNA
cttgtaagta

SEQ ID NO: 650 - DNA, 10 bp, synthetic construct
  source 1..10: mol_type = other DNA
catattgccg

SEQ ID NO: 651 - DNA, 10 bp, synthetic construct
  source 1..10: mol_type = other DNA
cttagaaagt

SEQ ID NO: 652 - DNA, 10 bp, synthetic construct
  source 1..10: mol_type = other DNA
atgttgtatt

SEQ ID NO: 653 - DNA, 10 bp, synthetic construct
  source 1..10: mol_type = other DNA
cgcattgaag

SEQ ID NO: 654 - DNA, 10 bp, synthetic construct
  source 1..10: mol_type = other DNA
ttatgttggt

SEQ ID NO: 655 - DNA, 10 bp, synthetic construct
  source 1..10: mol_type = other DNA
tcgcctcaga

SEQ ID NO: 656 - DNA, 10 bp, synthetic construct
  source 1..10: mol_type = other DNA
ttcgttgagg

SEQ ID NO: 657 - DNA, 10 bp, synthetic construct
  source 1..10: mol_type = other DNA
ggtgccgggc

SEQ ID NO: 658 - DNA, 10 bp, synthetic construct
  source 1..10: mol_type = other DNA
accattgtaa

SEQ ID NO: 659 - DNA, 10 bp, synthetic construct
  source 1..10: mol_type = other DNA
ttgattgtca

SEQ ID NO: 660 - DNA, 10 bp, synthetic construct
  source 1..10: mol_type = other DNA
cggctcacct

SEQ ID NO: 661 - DNA, 10 bp, synthetic construct
  source 1..10: mol_type = other DNA
ctatcacatg

SEQ ID NO: 662 - DNA, 10 bp, synthetic construct
  source 1..10: mol_type = other DNA
gtagacagaa

SEQ ID NO: 663 - DNA, 10 bp, synthetic construct
  source 1..10: mol_type = other DNA
cctttaccaa

SEQ ID NO: 664 - DNA, 10 bp, synthetic construct
  source 1..10: mol_type = other DNA
gcacatcgac

SEQ ID NO: 665 - DNA, 10 bp, synthetic construct
  source 1..10: mol_type = other DNA
tctcactttc

SEQ ID NO: 666 - DNA, 10 bp, synthetic construct
  source 1..10: mol_type = other DNA
ttcgagtact

SEQ ID NO: 667 - DNA, 10 bp, synthetic construct
  source 1..10: mol_type = other DNA
tagaagagca

SEQ ID NO: 668 - DNA, 10 bp, synthetic construct
  source 1..10: mol_type = other DNA
aaccccacca

SEQ ID NO: 669 - DNA, 10 bp, synthetic construct
  source 1..10: mol_type = other DNA
ctgtatcagt

SEQ ID NO: 670 - DNA, 10 bp, synthetic construct
  source 1..10: mol_type = other DNA
acataatgag

SEQ ID NO: 671 - DNA, 10 bp, synthetic construct
  source 1..10: mol_type = other DNA
agccttccgc

SEQ ID NO: 672 - DNA, 10 bp, synthetic construct
  source 1..10: mol_type = other DNA
cagtgctttt

SEQ ID NO: 673 - DNA, 10 bp, synthetic construct
  source 1..10: mol_type = other DNA
tagtccgtgt

SEQ ID NO: 674 - DNA, 10 bp, synthetic construct
  source 1..10: mol_type = other DNA
cggaatcggt

SEQ ID NO: 675 - DNA, 10 bp, synthetic construct
  source 1..10: mol_type = other DNA
cttgcggaga

SEQ ID NO: 676 - DNA, 10 bp, synthetic construct
  source 1..10: mol_type = other DNA
aaaaatttgg

SEQ ID NO: 677 - DNA, 10 bp, synthetic construct
  source 1..10: mol_type = other DNA
tgttttccgc

SEQ ID NO: 678 - DNA, 10 bp, synthetic construct
  source 1..10: mol_type = other DNA
atgctaggcg

SEQ ID NO: 679 - DNA, 10 bp, synthetic construct
  source 1..10: mol_type = other DNA
gactaatttc

SEQ ID NO: 680 - DNA, 10 bp, synthetic construct
  source 1..10: mol_type = other DNA
ctgtagtaac

SEQ ID NO: 681 - DNA, 10 bp, synthetic construct
  source 1..10: mol_type = other DNA
cggatgactt

SEQ ID NO: 682 - DNA, 10 bp, synthetic construct
  source 1..10: mol_type = other DNA
tcagagtgga

SEQ ID NO: 683 - DNA, 10 bp, synthetic construct
  source 1..10: mol_type = other DNA
caaaatagcg

SEQ ID NO: 684 - DNA, 10 bp, synthetic construct
  source 1..10: mol_type = other DNA
gaagaagaag

SEQ ID NO: 685 - DNA, 10 bp, synthetic construct
  source 1..10: mol_type = other DNA
cacccgcacg

SEQ ID NO: 686 - DNA, 10 bp, synthetic construct
  source 1..10: mol_type = other DNA
acgatgcccg

SEQ ID NO: 687 - DNA, 10 bp, synthetic construct
  source 1..10: mol_type = other DNA
cctactacac

SEQ ID NO: 688 - DNA, 10 bp, synthetic construct
  source 1..10: mol_type = other DNA
attgaaacaa

SEQ ID NO: 689 - DNA, 10 bp, synthetic construct
  source 1..10: mol_type = other DNA
gaccgaagat

SEQ ID NO: 690 - DNA, 10 bp, synthetic construct
  source 1..10: mol_type = other DNA
acggcctgaa

SEQ ID NO: 691 - DNA, 10 bp, synthetic construct
  source 1..10: mol_type = other DNA
aggggaggtc

SEQ ID NO: 692 - DNA, 10 bp, synthetic construct
  source 1..10: mol_type = other DNA
caatcaactt

SEQ ID NO: 693 - DNA, 10 bp, synthetic construct
  source 1..10: mol_type = other DNA
ggacaaccga

SEQ ID NO: 694 - DNA, 10 bp, synthetic construct
  source 1..10: mol_type = other DNA
tccctaaggc

SEQ ID NO: 695 - DNA, 10 bp, synthetic construct
  source 1..10: mol_type = other DNA
gttctacacg

SEQ ID NO: 696 - DNA, 10 bp, synthetic construct
  source 1..10: mol_type = other DNA
actaaccagt

SEQ ID NO: 697 - DNA, 10 bp, synthetic construct
  source 1..10: mol_type = other DNA
gaagctggat

SEQ ID NO: 698 - DNA, 10 bp, synthetic construct
  source 1..10: mol_type = other DNA
ggaaccatgg

SEQ ID NO: 699 - DNA, 10 bp, synthetic construct
  source 1..10: mol_type = other DNA
ctctacctgg

SEQ ID NO: 700 - DNA, 10 bp, synthetic construct
  source 1..10: mol_type = other DNA
taatgcctgc

SEQ ID NO: 701 - DNA, 10 bp, synthetic construct
  source 1..10: mol_type = other DNA
taaaggcaat

SEQ ID NO: 702 - DNA, 10 bp, synthetic construct
  source 1..10: mol_type = other DNA
cgcctgggaa

SEQ ID NO: 703 - DNA, 10 bp, synthetic construct
  source 1..10: mol_type = other DNA
tcttggggaa

SEQ ID NO: 704 - DNA, 10 bp, synthetic construct
  source 1..10: mol_type = other DNA
agagagagag

SEQ ID NO: 705 - DNA, 10 bp, synthetic construct
  source 1..10: mol_type = other DNA
gcgttggcgc

SEQ ID NO: 706 - DNA, 10 bp, synthetic construct
  source 1..10: mol_type = other DNA
ttacgacaga

SEQ ID NO: 707 - DNA, 10 bp, synthetic construct
  source 1..10: mol_type = other DNA
ggaactctta

SEQ ID NO: 708 - DNA, 10 bp, synthetic construct
  source 1..10: mol_type = other DNA
gattgtggag

SEQ ID NO: 709 - DNA, 10 bp, synthetic construct
  source 1..10: mol_type = other DNA
gggcactgat

SEQ ID NO: 710 - DNA, 10 bp, synthetic construct
  source 1..10: mol_type = other DNA
agacgcacca

SEQ ID NO: 711 - DNA, 10 bp, synthetic construct
  source 1..10: mol_type = other DNA
ccaattataa

SEQ ID NO: 712 - DNA, 10 bp, synthetic construct
  source 1..10: mol_type = other DNA
tagagacgca

SEQ ID NO: 713 - DNA, 10 bp, synthetic construct
  source 1..10: mol_type = other DNA
cctcttgtcg

SEQ ID NO: 714 - DNA, 10 bp, synthetic construct
  source 1..10: mol_type = other DNA
gaggaagctc

SEQ ID NO: 715 - DNA, 10 bp, synthetic construct
  source 1..10: mol_type = other DNA
agtcccgagt

SEQ ID NO: 716 - DNA, 10 bp, synthetic construct
  source 1..10: mol_type = other DNA
tgcttgcagt

SEQ ID NO: 717 - DNA, 10 bp, synthetic construct
  source 1..10: mol_type = other DNA
cccacttccc

SEQ ID NO: 718 - DNA, 10 bp, synthetic construct
  source 1..10: mol_type = other DNA
cgttgccgcg

SEQ ID NO: 719 - DNA, 10 bp, synthetic construct
  source 1..10: mol_type = other DNA
cccctggttc

SEQ ID NO: 720 - DNA, 10 bp, synthetic construct
  source 1..10: mol_type = other DNA
acgaccaata

SEQ ID NO: 721 - DNA, 10 bp, synthetic construct
  source 1..10: mol_type = other DNA
cttagggttc

SEQ ID NO: 722 - DNA, 10 bp, synthetic construct
  source 1..10: mol_type = other DNA
aaacatatca

SEQ ID NO: 723 - DNA, 10 bp, synthetic construct
  source 1..10: mol_type = other DNA
gggtcgtaga

SEQ ID NO: 724 - DNA, 10 bp, synthetic construct
  source 1..10: mol_type = other DNA
ctccgtagcg

SEQ ID NO: 725 - DNA, 10 bp, synthetic construct
  source 1..10: mol_type = other DNA
ctggtcataa

SEQ ID NO: 726 - DNA, 10 bp, synthetic construct
  source 1..10: mol_type = other DNA
ttgacagatc

SEQ ID NO: 727 - DNA, 10 bp, synthetic construct
  source 1..10: mol_type = other DNA
gagtaaagtc

SEQ ID NO: 728 - DNA, 10 bp, synthetic construct
  source 1..10: mol_type = other DNA
atatgggctt

SEQ ID NO: 729 - DNA, 10 bp, synthetic construct
  source 1..10: mol_type = other DNA
tacaactact

SEQ ID NO: 730 - DNA, 10 bp, synthetic construct
  source 1..10: mol_type = other DNA
aattcagccg

SEQ ID NO: 731 - DNA, 10 bp, synthetic construct
  source 1..10: mol_type = other DNA
gattgtacta

SEQ ID NO: 732 - DNA, 10 bp, synthetic construct
  source 1..10: mol_type = other DNA
tcgtaatgcg

SEQ ID NO: 733 - DNA, 10 bp, synthetic construct
  source 1..10: mol_type = other DNA
cgataactgc

SEQ ID NO: 734 - DNA, 10 bp, synthetic construct
  source 1..10: mol_type = other DNA
aacttggcgg

SEQ ID NO: 735 - DNA, 10 bp, synthetic construct
  source 1..10: mol_type = other DNA
cgtggatgta

SEQ ID NO: 736 - DNA, 10 bp, synthetic construct
  source 1..10: mol_type = other DNA
ccttcccgaa

SEQ ID NO: 737 - DNA, 10 bp, synthetic construct
  source 1..10: mol_type = other DNA
ctaaacccgt

SEQ ID NO: 738 - DNA, 10 bp, synthetic construct
  source 1..10: mol_type = other DNA
caacattccc

SEQ ID NO: 739 - DNA, 10 bp, synthetic construct
  source 1..10: mol_type = other DNA
cttaccctct

SEQ ID NO: 740 - DNA, 10 bp, synthetic construct
  source 1..10: mol_type = other DNA
ggaaagttct

SEQ ID NO: 741 - DNA, 10 bp, synthetic construct
  source 1..10: mol_type = other DNA
cggattggct

SEQ ID NO: 742 - DNA, 10 bp, synthetic construct
  source 1..10: mol_type = other DNA
aatgtagggc

SEQ ID NO: 743 - DNA, 10 bp, synthetic construct
  source 1..10: mol_type = other DNA
aatgaatcgc

SEQ ID NO: 744 - DNA, 10 bp, synthetic construct
  source 1..10: mol_type = other DNA
atcatacacc

SEQ ID NO: 745 - DNA, 10 bp, synthetic construct
  source 1..10: mol_type = other DNA
agttgggcag

SEQ ID NO: 746 - DNA, 10 bp, synthetic construct
  source 1..10: mol_type = other DNA
agaagaaggg

SEQ ID NO: 747 - DNA, 10 bp, synthetic construct
  source 1..10: mol_type = other DNA
gcgtgcgcta

SEQ ID NO: 748 - DNA, 10 bp, synthetic construct
  source 1..10: mol_type = other DNA
ccccgataaa

SEQ ID NO: 749 - DNA, 10 bp, synthetic construct
  source 1..10: mol_type = other DNA
taccaagtgc

SEQ ID NO: 750 - DNA, 10 bp, synthetic construct
  source 1..10: mol_type = other DNA
tgtgttttcg

SEQ ID NO: 751 - DNA, 10 bp, synthetic construct
  source 1..10: mol_type = other DNA
cccagatgtc

SEQ ID NO: 752 - DNA, 10 bp, synthetic construct
  source 1..10: mol_type = other DNA
gcgagcttcc

SEQ ID NO: 753 - DNA, 10 bp, synthetic construct
  source 1..10: mol_type = other DNA
gtgtcacgta

SEQ ID NO: 754 - DNA, 10 bp, synthetic construct
  source 1..10: mol_type = other DNA
ataggccgag

SEQ ID NO: 755 - DNA, 10 bp, synthetic construct
  source 1..10: mol_type = other DNA
gagctaccag

SEQ ID NO: 756 - DNA, 10 bp, synthetic construct
  source 1..10: mol_type = other DNA
cgcggcggag

SEQ ID NO: 757 - DNA, 10 bp, synthetic construct
  source 1..10: mol_type = other DNA
tcttgcacga

SEQ ID NO: 758 - DNA, 10 bp, synthetic construct
  source 1..10: mol_type = other DNA
tgccctaaag

SEQ ID NO: 759 - DNA, 10 bp, synthetic construct
  source 1..10: mol_type = other DNA
ttgcgctttg

SEQ ID NO: 760 - DNA, 10 bp, synthetic construct
  source 1..10: mol_type = other DNA
catataaagg

SEQ ID NO: 761 - DNA, 10 bp, synthetic construct
  source 1..10: mol_type = other DNA
aatagcgaat

SEQ ID NO: 762 - DNA, 10 bp, synthetic construct
  source 1..10: mol_type = other DNA
tacgctaagg

SEQ ID NO: 763 - DNA, 10 bp, synthetic construct
  source 1..10: mol_type = other DNA
acttagttcg

SEQ ID NO: 764 - DNA, 10 bp, synthetic construct
  source 1..10: mol_type = other DNA
cgtgcggaac

SEQ ID NO: 765 - DNA, 10 bp, synthetic construct
  source 1..10: mol_type = other DNA
acccgattcg

SEQ ID NO: 766 - DNA, 10 bp, synthetic construct
  source 1..10: mol_type = other DNA
tgcagagttt

SEQ ID NO: 767 - DNA, 10 bp, synthetic construct
  source 1..10: mol_type = other DNA
gaatcattag

SEQ ID NO: 768 - DNA, 10 bp, synthetic construct
  source 1..10: mol_type = other DNA
agtacactgg

SEQ ID NO: 769 - DNA, 10 bp, synthetic construct
  source 1..10: mol_type = other DNA
ttgtgcggtt

SEQ ID NO: 770 - DNA, 10 bp, synthetic construct
  source 1..10: mol_type = other DNA
atgacatgca

SEQ ID NO: 771 - DNA, 10 bp, synthetic construct
  source 1..10: mol_type = other DNA
ttctcggacg

SEQ ID NO: 772 - DNA, 10 bp, synthetic construct
  source 1..10: mol_type = other DNA
agattgaaga

SEQ ID NO: 773 - DNA, 10 bp, synthetic construct
  source 1..10: mol_type = other DNA
ggcggactgt

SEQ ID NO: 774 - DNA, 10 bp, synthetic construct
  source 1..10: mol_type = other DNA
tttatggtaa

SEQ ID NO: 775 - DNA, 10 bp, synthetic construct
  source 1..10: mol_type = other DNA
cagtagggtg

SEQ ID NO: 776 - DNA, 10 bp, synthetic construct
  source 1..10: mol_type = other DNA
gacaggcaag

SEQ ID NO: 777 - DNA, 10 bp, synthetic construct
  source 1..10: mol_type = other DNA
gatgtgtcgt

SEQ ID NO: 778 - DNA, 10 bp, synthetic construct
  source 1..10: mol_type = other DNA
acttgacgga

SEQ ID NO: 779 - DNA, 10 bp, synthetic construct
  source 1..10: mol_type = other DNA
aagtccgaaa

SEQ ID NO: 780 - DNA, 10 bp, synthetic construct
  source 1..10: mol_type = other DNA
tgggtgtagg

SEQ ID NO: 781 - DNA, 10 bp, synthetic construct
  source 1..10: mol_type = other DNA
acttaccgcg

SEQ ID NO: 782 - DNA, 10 bp, synthetic construct
  source 1..10: mol_type = other DNA
ctgtgcaccc

SEQ ID NO: 783 - DNA, 10 bp, synthetic construct
  source 1..10: mol_type = other DNA
attgctctct

SEQ ID NO: 784 - DNA, 10 bp, synthetic construct
  source 1..10: mol_type = other DNA
cagaagacaa

SEQ ID NO: 785 - DNA, 10 bp, synthetic construct
  source 1..10: mol_type = other DNA
ttacgctata

SEQ ID NO: 786 - DNA, 10 bp, synthetic construct
  source 1..10: mol_type = other DNA
acgtggaaat

SEQ ID NO: 787 - DNA, 10 bp, synthetic construct
  source 1..10: mol_type = other DNA
tgaggctggt

SEQ ID NO: 788 - DNA, 10 bp, synthetic construct
  source 1..10: mol_type = other DNA
attatgagat

SEQ ID NO: 789 - DNA, 10 bp, synthetic construct
  source 1..10: mol_type = other DNA
gacttgtagt

SEQ ID NO: 790 - DNA, 10 bp, synthetic construct
  source 1..10: mol_type = other DNA
tcgctgagga

SEQ ID NO: 791 - DNA, 10 bp, synthetic construct
  source 1..10: mol_type = other DNA
cccaactcta

SEQ ID NO: 792 - DNA, 10 bp, synthetic construct
  source 1..10: mol_type = other DNA
gatagggagg

SEQ ID NO: 793 - DNA, 10 bp, synthetic construct
  source 1..10: mol_type = other DNA
tagaaatcag

SEQ ID NO: 794 - DNA, 10 bp, synthetic construct
  source 1..10: mol_type = other DNA
gtcgctagaa

SEQ ID NO: 795 - DNA, 10 bp, synthetic construct
  source 1..10: mol_type = other DNA
aaaatagaaa

SEQ ID NO: 796 - DNA, 10 bp, synthetic construct
  source 1..10: mol_type = other DNA
gctcctgggt

SEQ ID NO: 797 - DNA, 10 bp, synthetic construct
  source 1..10: mol_type = other DNA
cgcgctcgcg

SEQ ID NO: 798 - DNA, 10 bp, synthetic construct
  source 1..10: mol_type = other DNA
ggcaaacgca

SEQ ID NO: 799 - DNA, 10 bp, synthetic construct
  source 1..10: mol_type = other DNA
tttactacct

SEQ ID NO: 800 - DNA, 10 bp, synthetic construct
  source 1..10: mol_type = other DNA
atcctaaact

SEQ ID NO: 801 - DNA, 10 bp, synthetic construct
  source 1..10: mol_type = other DNA
ctccgtatgt

SEQ ID NO: 802 - DNA, 10 bp, synthetic construct
  source 1..10: mol_type = other DNA
tatcgtccag

SEQ ID NO: 803 - DNA, 10 bp, synthetic construct
  source 1..10: mol_type = other DNA
gccggcggta

SEQ ID NO: 804 - DNA, 10 bp, synthetic construct
  source 1..10: mol_type = other DNA
tgctccattt

SEQ ID NO: 805 - DNA, 10 bp, synthetic construct
  source 1..10: mol_type = other DNA
tggctgttgt

SEQ ID NO: 806 - DNA, 10 bp, synthetic construct
  source 1..10: mol_type = other DNA
tactgcgcaa

SEQ ID NO: 807 - DNA, 10 bp, synthetic construct
  source 1..10: mol_type = other DNA
tatacggctt

SEQ ID NO: 808 - DNA, 10 bp, synthetic construct
  source 1..10: mol_type = other DNA
ggttattacc

SEQ ID NO: 809 - DNA, 10 bp, synthetic construct
  source 1..10: mol_type = other DNA
atcaggagga

SEQ ID NO: 810 - DNA, 10 bp, synthetic construct
  source 1..10: mol_type = other DNA
ctattgccag

SEQ ID NO: 811 - DNA, 10 bp, synthetic construct
  source 1..10: mol_type = other DNA
acgtacacac

SEQ ID NO: 812 - DNA, 10 bp, synthetic construct
  source 1..10: mol_type = other DNA
cagcctagct

SEQ ID NO: 813 - DNA, 10 bp, synthetic construct
  source 1..10: mol_type = other DNA
gaaaaacaac

SEQ ID NO: 814 - DNA, 10 bp, synthetic construct
  source 1..10: mol_type = other DNA
cgttcagtta

SEQ ID NO: 815 - DNA, 10 bp, synthetic construct
  source 1..10: mol_type = other DNA
caatcagaat

SEQ ID NO: 816 - DNA, 10 bp, synthetic construct
  source 1..10: mol_type = other DNA
gggctactct

SEQ ID NO: 817 - DNA, 10 bp, synthetic construct
  source 1..10: mol_type = other DNA
ccccattggg

SEQ ID NO: 818 - DNA, 10 bp, synthetic construct
  source 1..10: mol_type = other DNA
tagggaacgg

SEQ ID NO: 819 - DNA, 10 bp, synthetic construct
  source 1..10: mol_type = other DNA
cagctgatac

SEQ ID NO: 820 - DNA, 10 bp, synthetic construct
  source 1..10: mol_type = other DNA
attcctgtga

SEQ ID NO: 821 - DNA, 10 bp, synthetic construct
  source 1..10: mol_type = other DNA
tcagagccgt

SEQ ID NO: 822 - DNA, 10 bp, synthetic construct
  source 1..10: mol_type = other DNA
catgaaaagc

SEQ ID NO: 823 - DNA, 10 bp, synthetic construct
  source 1..10: mol_type = other DNA
tgacctgtga

SEQ ID NO: 824 - DNA, 10 bp, synthetic construct
  source 1..10: mol_type = other DNA
gcattagcag

SEQ ID NO: 825 - DNA, 10 bp, synthetic construct
  source 1..10: mol_type = other DNA
gacagaacca

SEQ ID NO: 826 - DNA, 10 bp, synthetic construct
  source 1..10: mol_type = other DNA
tccagtatat

SEQ ID NO: 827 - DNA, 10 bp, synthetic construct
  source 1..10: mol_type = other DNA
tgttccgcta

SEQ ID NO: 828 - DNA, 10 bp, synthetic construct
  source 1..10: mol_type = other DNA
gatatccatt

SEQ ID NO: 829 - DNA, 10 bp, synthetic construct
  source 1..10: mol_type = other DNA
catatggacc

SEQ ID NO: 830 - DNA, 10 bp, synthetic construct
  source 1..10: mol_type = other DNA
gatatagtaa

SEQ ID NO: 831 - DNA, 10 bp, synthetic construct
  source 1..10: mol_type = other DNA
cacctttttt

SEQ ID NO: 832 - DNA, 10 bp, synthetic construct
  source 1..10: mol_type = other DNA
agcttgcggg

SEQ ID NO: 833 - DNA, 10 bp, synthetic construct
  source 1..10: mol_type = other DNA
cgcacaggga

SEQ ID NO: 834 - DNA, 10 bp, synthetic construct
  source 1..10: mol_type = other DNA
tctgggtgct

SEQ ID NO: 835 - DNA, 10 bp, synthetic construct
  source 1..10: mol_type = other DNA
tgagtcgttt

SEQ ID NO: 836 - DNA, 10 bp, synthetic construct
  source 1..10: mol_type = other DNA
ttacaatgtg

SEQ ID NO: 837 - DNA, 10 bp, synthetic construct
  source 1..10: mol_type = other DNA
cttgcaaaca

SEQ ID NO: 838 - DNA, 10 bp, synthetic construct
  source 1..10: mol_type = other DNA
tgtcgagctg

SEQ ID NO: 839 - DNA, 10 bp, synthetic construct
  source 1..10: mol_type = other DNA
actttaacct

SEQ ID NO: 840 - DNA, 10 bp, synthetic construct
  source 1..10: mol_type = other DNA
atataagtgc

SEQ ID NO: 841 - DNA, 10 bp, synthetic construct
  source 1..10: mol_type = other DNA
ggaagggcgt

SEQ ID NO: 842 - DNA, 10 bp, synthetic construct
  source 1..10: mol_type = other DNA
tttgacttga

SEQ ID NO: 843 - DNA, 10 bp, synthetic construct
  source 1..10: mol_type = other DNA
gtataaacgg

SEQ ID NO: 844 - DNA, 10 bp, synthetic construct
  source 1..10: mol_type = other DNA
taaccggatg

SEQ ID NO: 845 - DNA, 10 bp, synthetic construct
  source 1..10: mol_type = other DNA
ttctcatcag

SEQ ID NO: 846 - DNA, 10 bp, synthetic construct
  source 1..10: mol_type = other DNA
ctcggttacg

SEQ ID NO: 847 - DNA, 10 bp, synthetic construct
  source 1..10: mol_type = other DNA
atatggttct

SEQ ID NO: 848 - DNA, 10 bp, synthetic construct
  source 1..10: mol_type = other DNA
cgcccccgaa

SEQ ID NO: 849 - DNA, 10 bp, synthetic construct
  source 1..10: mol_type = other DNA
acctcgatcg

SEQ ID NO: 850 - DNA, 10 bp, synthetic construct
  source 1..10: mol_type = other DNA
ctcgaataat

SEQ ID NO: 851 - DNA, 10 bp, synthetic construct
  source 1..10: mol_type = other DNA
gcccgagctt

SEQ ID NO: 852 - DNA, 10 bp, synthetic construct
  source 1..10: mol_type = other DNA
aacagtcaac

SEQ ID NO: 853 - DNA, 10 bp, synthetic construct
  source 1..10: mol_type = other DNA
ctggaacctc

SEQ ID NO: 854 - DNA, 10 bp, synthetic construct
  source 1..10: mol_type = other DNA
aataacgggg

SEQ ID NO: 855 - DNA, 10 bp, synthetic construct
  source 1..10: mol_type = other DNA
acgccccact

SEQ ID NO: 856 - DNA, 10 bp, synthetic construct
  source 1..10: mol_type = other DNA
ggcaacatga

SEQ ID NO: 857 - DNA, 10 bp, synthetic construct
  source 1..10: mol_type = other DNA
gctatttcgc

SEQ ID NO: 858 - DNA, 10 bp, synthetic construct
  source 1..10: mol_type = other DNA
ttccacttta

SEQ ID NO: 859 - DNA, 10 bp, synthetic construct
  source 1..10: mol_type = other DNA
gccgatggat

SEQ ID NO: 860 - DNA, 10 bp, synthetic construct
  source 1..10: mol_type = other DNA
aagttggtaa

SEQ ID NO: 861 - DNA, 10 bp, synthetic construct
  source 1..10: mol_type = other DNA
cactagctag

SEQ ID NO: 862 - DNA, 10 bp, synthetic construct
  source 1..10: mol_type = other DNA
acatgcccct

SEQ ID NO: 863 - DNA, 10 bp, synthetic construct
  source 1..10: mol_type = other DNA
ttcattactc

SEQ ID NO: 864 - DNA, 10 bp, synthetic construct
  source 1..10: mol_type = other DNA
ggtttaatat

SEQ ID NO: 865 - DNA, 10 bp, synthetic construct
  source 1..10: mol_type = other DNA
cctgcagtga

SEQ ID NO: 866 - DNA, 10 bp, synthetic construct
  source 1..10: mol_type = other DNA
tctttaagtt

SEQ ID NO: 867 - DNA, 10 bp, synthetic construct
  source 1..10: mol_type = other DNA
tggcgatcga

SEQ ID NO: 868 - DNA, 10 bp, synthetic construct
  source 1..10: mol_type = other DNA
ctttttagct

SEQ ID NO: 869 - DNA, 10 bp, synthetic construct
  source 1..10: mol_type = other DNA
cccagtctct

SEQ ID NO: 870 - DNA, 10 bp, synthetic construct
  source 1..10: mol_type = other DNA
aaatgtttcg

SEQ ID NO: 871 - DNA, 10 bp, synthetic construct
  source 1..10: mol_type = other DNA
atataagacg

SEQ ID NO: 872 - DNA, 10 bp, synthetic construct
  source 1..10: mol_type = other DNA
tcactttaca

SEQ ID NO: 873 - DNA, 10 bp, synthetic construct
  source 1..10: mol_type = other DNA
cctggcgccc

SEQ ID NO: 874 - DNA, 10 bp, synthetic construct
  source 1..10: mol_type = other DNA
ggattactgg

SEQ ID NO: 875 - DNA, 10 bp, synthetic construct
  source 1..10: mol_type = other DNA
gaatgatctt

SEQ ID NO: 876 - DNA, 10 bp, synthetic construct
  source 1..10: mol_type = other DNA
gctcggatcg

SEQ ID NO: 877 - DNA, 10 bp, synthetic construct
  source 1..10: mol_type = other DNA
cagctgcgag

SEQ ID NO: 878 - DNA, 10 bp, synthetic construct
  source 1..10: mol_type = other DNA
acccttacta

SEQ ID NO: 879 - DNA, 10 bp, synthetic construct
  source 1..10: mol_type = other DNA
aggtgaaact

SEQ ID NO: 880 - DNA, 10 bp, synthetic construct
  source 1..10: mol_type = other DNA
cgaatttgat

SEQ ID NO: 881 - DNA, 10 bp, synthetic construct
  source 1..10: mol_type = other DNA
cgctgtgcgg

SEQ ID NO: 882 - DNA, 10 bp, synthetic construct
  source 1..10: mol_type = other DNA
ttaccgcacc

SEQ ID NO: 883 - DNA, 10 bp, synthetic construct
  source 1..10: mol_type = other DNA
ggaatcttaa

SEQ ID NO: 884 - DNA, 10 bp, synthetic construct
  source 1..10: mol_type = other DNA
ctcaacaccc

SEQ ID NO: 885 - DNA, 10 bp, synthetic construct
  source 1..10: mol_type = other DNA
cgtgcccttg

SEQ ID NO: 886 - DNA, 10 bp, synthetic construct
  source 1..10: mol_type = other DNA
gcaggctcga

SEQ ID NO: 887 - DNA, 10 bp, synthetic construct
  source 1..10: mol_type = other DNA
accaacgaag

SEQ ID NO: 888 - DNA, 10 bp, synthetic construct
  source 1..10: mol_type = other DNA
cctgtaattt

SEQ ID NO: 889 - DNA, 10 bp, synthetic construct
  source 1..10: mol_type = other DNA
gggtgggatg

SEQ ID NO: 890 - DNA, 10 bp, synthetic construct
  source 1..10: mol_type = other DNA
ttgctcaccg

SEQ ID NO: 891 - DNA, 10 bp, synthetic construct
  source 1..10: mol_type = other DNA
ttacgaccac

SEQ ID NO: 892 - DNA, 10 bp, synthetic construct
  source 1..10: mol_type = other DNA
ttttctaacc

SEQ ID NO: 893 - DNA, 10 bp, synthetic construct
  source 1..10: mol_type = other DNA
gctttagata

SEQ ID NO: 894 - DNA, 10 bp, synthetic construct
  source 1..10: mol_type = other DNA
cacgtattgg

SEQ ID NO: 895 - DNA, 10 bp, synthetic construct
  source 1..10: mol_type = other DNA
aaatatctcc

SEQ ID NO: 896 - DNA, 10 bp, synthetic construct
  source 1..10: mol_type = other DNA
gctggaaaac

SEQ ID NO: 897 - DNA, 10 bp, synthetic construct
  source 1..10: mol_type = other DNA
gagcgcatta

SEQ ID NO: 898 - DNA, 10 bp, synthetic construct
  source 1..10: mol_type = other DNA
gtggaggggt

SEQ ID NO: 899 - DNA, 10 bp, synthetic construct
  source 1..10: mol_type = other DNA
tccactggga

SEQ ID NO: 900 - DNA, 10 bp, synthetic construct
  source 1..10: mol_type = other DNA
caatagcgga

SEQ ID NO: 901 - DNA, 10 bp, synthetic construct
  source 1..10: mol_type = other DNA
catctagttt

SEQ ID NO: 902 - DNA, 10 bp, synthetic construct
  source 1..10: mol_type = other DNA
gaagttccgg

SEQ ID NO: 903 - DNA, 10 bp, synthetic construct
  source 1..10: mol_type = other DNA
agcgagattc

SEQ ID NO: 904 - DNA, 10 bp, synthetic construct
  source 1..10: mol_type = other DNA
ttaaggtcgg

SEQ ID NO: 905 - DNA, 10 bp, synthetic construct
  source 1..10: mol_type = other DNA
aatggttagg

SEQ ID NO: 906 - DNA, 10 bp, synthetic construct
  source 1..10: mol_type = other DNA
cgttattata

SEQ ID NO: 907 - DNA, 10 bp, synthetic construct
  source 1..10: mol_type = other DNA
acggaaagga

SEQ ID NO: 908 - DNA, 10 bp, synthetic construct
  source 1..10: mol_type = other DNA
ccttgtcccg

SEQ ID NO: 909 - DNA, 10 bp, synthetic construct
  source 1..10: mol_type = other DNA
atactttttt

SEQ ID NO: 910 - DNA, 10 bp, synthetic construct
  source 1..10: mol_type = other DNA
ctgggtctgg

SEQ ID NO: 911 - DNA, 10 bp, synthetic construct
  source 1..10: mol_type = other DNA
aaccattgcg

SEQ ID NO: 912 - DNA, 10 bp, synthetic construct
  source 1..10: mol_type = other DNA
agaccgggcc

SEQ ID NO: 913 - DNA, 10 bp, synthetic construct
  source 1..10: mol_type = other DNA
tgggacacac

SEQ ID NO: 914 - DNA, 10 bp, synthetic construct
  source 1..10: mol_type = other DNA
tgcgcagttg

SEQ ID NO: 915 - DNA, 10 bp, synthetic construct
  source 1..10: mol_type = other DNA
cgttcgcctt

SEQ ID NO: 916 - DNA, 10 bp, synthetic construct
  source 1..10: mol_type = other DNA
tctcactcgt

SEQ ID NO: 917 - DNA, 10 bp, synthetic construct
  source 1..10: mol_type = other DNA
acaccgacgt

SEQ ID NO: 918 - DNA, 10 bp, synthetic construct
  source 1..10: mol_type = other DNA
ttcagcccct

SEQ ID NO: 919 - DNA, 10 bp, synthetic construct
  source 1..10: mol_type = other DNA
aggcgactaa

SEQ ID NO: 920 - DNA, 10 bp, synthetic construct
  source 1..10: mol_type = other DNA
tgctatcaag

SEQ ID NO: 921 - DNA, 10 bp, synthetic construct
  source 1..10: mol_type = other DNA
gtccagtagc

SEQ ID NO: 922 - DNA, 10 bp, synthetic construct
  source 1..10: mol_type = other DNA
cgtgtgggcg

SEQ ID NO: 923 - DNA, 10 bp, synthetic construct
  source 1..10: mol_type = other DNA
gtggttctcc

SEQ ID NO: 924 - DNA, 10 bp, synthetic construct
  source 1..10: mol_type = other DNA
gcagccgacg

SEQ ID NO: 925 - DNA, 10 bp, synthetic construct
  source 1..10: mol_type = other DNA
gctgtccacg

SEQ ID NO: 926 - DNA, 10 bp, synthetic construct
  source 1..10: mol_type = other DNA
cgacactcat

SEQ ID NO: 927 - DNA, 10 bp, synthetic construct
  source 1..10: mol_type = other DNA
catggcacct

SEQ ID NO: 928 - DNA, 10 bp, synthetic construct
  source 1..10: mol_type = other DNA
tgtgacgtgt

SEQ ID NO: 929 - DNA, 10 bp, synthetic construct
  source 1..10: mol_type = other DNA
tttggactaa

SEQ ID NO: 930 - DNA, 10 bp, synthetic construct
  source 1..10: mol_type = other DNA
ttcatgcccg

SEQ ID NO: 931 - DNA, 10 bp, synthetic construct
  source 1..10: mol_type = other DNA
ttgatcgtgg

SEQ ID NO: 932 - DNA, 10 bp, synthetic construct
  source 1..10: mol_type = other DNA
tagcatagga

SEQ ID NO: 933 - DNA, 10 bp, synthetic construct
  source 1..10: mol_type = other DNA
gtagttgcaa

SEQ ID NO: 934 - DNA, 10 bp, synthetic construct
  source 1..10: mol_type = other DNA
gggacagcta

SEQ ID NO: 935 - DNA, 10 bp, synthetic construct
  source 1..10: mol_type = other DNA
aaacccccaa

SEQ ID NO: 936 - DNA, 10 bp, synthetic construct
  source 1..10: mol_type = other DNA
actctcacaa

SEQ ID NO: 937 - DNA, 10 bp, synthetic construct
  source 1..10: mol_type = other DNA
atcattgcca

SEQ ID NO: 938 - DNA, 10 bp, synthetic construct
  source 1..10: mol_type = other DNA
ccagtttgcg

SEQ ID NO: 939 - DNA, 10 bp, synthetic construct
  source 1..10: mol_type = other DNA
acattagtca

SEQ ID NO: 940 - DNA, 10 bp, synthetic construct
  source 1..10: mol_type = other DNA
ctccagggta

SEQ ID NO: 941 - DNA, 10 bp, synthetic construct
  source 1..10: mol_type = other DNA
gaagggccaa

SEQ ID NO: 942 - DNA, 10 bp, synthetic construct
  source 1..10: mol_type = other DNA
cagtctcccc

SEQ ID NO: 943 - DNA, 10 bp, synthetic construct
  source 1..10: mol_type = other DNA
gagacattcc

SEQ ID NO: 944 - DNA, 10 bp, synthetic construct
  source 1..10: mol_type = other DNA
aacggtgttg

SEQ ID NO: 945 - DNA, 10 bp, synthetic construct
  source 1..10: mol_type = other DNA
agcattatca

SEQ ID NO: 946 - DNA, 10 bp, synthetic construct
  source 1..10: mol_type = other DNA
ctataccgag

SEQ ID NO: 947 - DNA, 10 bp, synthetic construct
  source 1..10: mol_type = other DNA
aactggatca

SEQ ID NO: 948 - DNA, 10 bp, synthetic construct
  source 1..10: mol_type = other DNA
gtcttgtcgg

SEQ ID NO: 949 - DNA, 10 bp, synthetic construct
  source 1..10: mol_type = other DNA
gacgagccgc

SEQ ID NO: 950 - DNA, 10 bp, synthetic construct
  source 1..10: mol_type = other DNA
ggaacactgt

SEQ ID NO: 951 - DNA, 10 bp, synthetic construct
  source 1..10: mol_type = other DNA
taaatgcgtt

SEQ ID NO: 952 - DNA, 10 bp, synthetic construct
  source 1..10: mol_type = other DNA
gcgaacacag

SEQ ID NO: 953 - DNA, 10 bp, synthetic construct
  source 1..10: mol_type = other DNA
ttctctcaac

SEQ ID NO: 954 - DNA, 10 bp, synthetic construct
  source 1..10: mol_type = other DNA
gtcgtactga

SEQ ID NO: 955 - DNA, 10 bp, synthetic construct
  source 1..10: mol_type = other DNA
tgtggcgtaa

SEQ ID NO: 956 - DNA, 10 bp, synthetic construct
  source 1..10: mol_type = other DNA
tgagcggcgt

SEQ ID NO: 957 - DNA, 10 bp, synthetic construct
  source 1..10: mol_type = other DNA
cctcgtgaac

SEQ ID NO: 958 - DNA, 10 bp, synthetic construct
  source 1..10: mol_type = other DNA
gagcaatgaa

SEQ ID NO: 959 - DNA, 10 bp, synthetic construct
  source 1..10: mol_type = other DNA
cgagacctaa

SEQ ID NO: 960 - DNA, 10 bp, synthetic construct
  source 1..10: mol_type = other DNA
aactgagcgc

SEQ ID NO: 961 - DNA, 10 bp, synthetic construct
  source 1..10: mol_type = other DNA
taaagctcgt

SEQ ID NO: 962 - DNA, 10 bp, synthetic construct
  source 1..10: mol_type = other DNA
ctctttacgt

SEQ ID NO: 963 - DNA, 10 bp, synthetic construct
  source 1..10: mol_type = other DNA
ccccgtggaa

SEQ ID NO: 964 - DNA, 10 bp, synthetic construct
  source 1..10: mol_type = other DNA
tcggttcgtc

SEQ ID NO: 965 - DNA, 10 bp, synthetic construct
  source 1..10: mol_type = other DNA
ctgcttacac

SEQ ID NO: 966 - DNA, 10 bp, synthetic construct
  source 1..10: mol_type = other DNA
acaccgtaat

SEQ ID NO: 967 - DNA, 10 bp, synthetic construct
  source 1..10: mol_type = other DNA
cctggtcggc

SEQ ID NO: 968 - DNA, 10 bp, synthetic construct
  source 1..10: mol_type = other DNA
ggttatttgg

SEQ ID NO: 969 - DNA, 10 bp, synthetic construct
  source 1..10: mol_type = other DNA
gcaactgagt

SEQ ID NO: 970 - DNA, 10 bp, synthetic construct
  source 1..10: mol_type = other DNA
ataaggcctc

SEQ ID NO: 971 - DNA, 10 bp, synthetic construct
  source 1..10: mol_type = other DNA
cgtgcgaagg

SEQ ID NO: 972 - DNA, 10 bp, synthetic construct
  source 1..10: mol_type = other DNA
gtcacacact

SEQ ID NO: 973 - DNA, 10 bp, synthetic construct
  source 1..10: mol_type = other DNA
catacggcaa

SEQ ID NO: 974 - DNA, 10 bp, synthetic construct
  source 1..10: mol_type = other DNA
gaactgccca

SEQ ID NO: 975 - DNA, 10 bp, synthetic construct
  source 1..10: mol_type = other DNA
aatatgtgaa

SEQ ID NO: 976 - DNA, 10 bp, synthetic construct
  source 1..10: mol_type = other DNA
ccgatcctgt

SEQ ID NO: 977 - DNA, 10 bp, synthetic construct
  source 1..10: mol_type = other DNA
caaagagcct

SEQ ID NO: 978 - DNA, 10 bp, synthetic construct
  source 1..10: mol_type = other DNA
taacttagag

SEQ ID NO: 979 - DNA, 10 bp, synthetic construct
  source 1..10: mol_type = other DNA
cagcatgtag

SEQ ID NO: 980 - DNA, 10 bp, synthetic construct
  source 1..10: mol_type = other DNA
ccccatgcag

SEQ ID NO: 981 - DNA, 10 bp, synthetic construct
  source 1..10: mol_type = other DNA
tctgaaccac

SEQ ID NO: 982 - DNA, 10 bp, synthetic construct
  source 1..10: mol_type = other DNA
gcgtgcaaaa

SEQ ID NO: 983 - DNA, 10 bp, synthetic construct
  source 1..10: mol_type = other DNA
gctagtaccg

SEQ ID NO: 984 - DNA, 10 bp, synthetic construct
  source 1..10: mol_type = other DNA
tttcccgcgc

SEQ ID NO: 985 - DNA, 10 bp, synthetic construct
  source 1..10: mol_type = other DNA
ccttagtagg

SEQ ID NO: 986 - DNA, 10 bp, synthetic construct
  source 1..10: mol_type = other DNA
ttgtgtcttg

SEQ ID NO: 987 - DNA, 10 bp, synthetic construct
  source 1..10: mol_type = other DNA
gcaacgaagc

SEQ ID NO: 988 - DNA, 10 bp, synthetic construct
  source 1..10: mol_type = other DNA
tgaaaccctt

SEQ ID NO: 989 - DNA, 10 bp, synthetic construct
  source 1..10: mol_type = other DNA
ttctacgatc

SEQ ID NO: 990 - DNA, 10 bp, synthetic construct
  source 1..10: mol_type = other DNA
attaaaggtg

SEQ ID NO: 991 - DNA, 10 bp, synthetic construct
  source 1..10: mol_type = other DNA
tatctaacgg

SEQ ID NO: 992 - DNA, 10 bp, synthetic construct
  source 1..10: mol_type = other DNA
agtgctcctg

SEQ ID NO: 993 - DNA, 10 bp, synthetic construct
  source 1..10: mol_type = other DNA
ccgtccctct

SEQ ID NO: 994 - DNA, 10 bp, synthetic construct
  source 1..10: mol_type = other DNA
ctaacgagcg

SEQ ID NO: 995 - DNA, 10 bp, synthetic construct
  source 1..10: mol_type = other DNA
aagtccggct

SEQ ID NO: 996 - DNA, 10 bp, synthetic construct
  source 1..10: mol_type = other DNA
ggcgtataag

SEQ ID NO: 997 - DNA, 10 bp, synthetic construct
  source 1..10: mol_type = other DNA
agatattagg

SEQ ID NO: 998 - DNA, 10 bp, synthetic construct
  source 1..10: mol_type = other DNA
tcctaacagc

SEQ ID NO: 999 - DNA, 10 bp, synthetic construct
  source 1..10: mol_type = other DNA
gaggatacgc

SEQ ID NO: 1000 - DNA, 10 bp, synthetic construct
  source 1..10: mol_type = other DNA
cgctctttaa

SEQ ID NO: 1001 - DNA, 21 bp, synthetic construct
  source 1..21: mol_type = other DNA
  modified_base 1: mod_base = OTHER; note = Nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
agacgtgtgctcttccgatct

SEQ ID NO: 1002 - DNA, 20 bp, synthetic construct
  source 1..20: mol_type = other DNA
  modified_base 17: mod_base = OTHER; note = Nucleotide with phosphorothioate linkage
  modified_base 18: mod_base = OTHER; note = Nucleotide with phosphorothioate linkage
  modified_base 19: mod_base = OTHER; note = Nucleotide with phosphorothioate linkage
agatcggaagagcgtcgtgt

SEQ ID NO: 1003 - DNA, 61 bp, synthetic construct
  source 1..61: mol_type = other DNA
  modified_base 1: mod_base = OTHER; note = 5' Amino Modifier C6 modified nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  modified_base 58: mod_base = OTHER; note = Nucleotide with phosphorothioate linkage
  modified_base 59: mod_base = OTHER; note = Nucleotide with phosphorothioate linkage
  modified_base 60: mod_base = OTHER; note = Nucleotide with phosphorothioate linkage
agacgtgtgctcttccgatctgctacataatnnnnnnnnnnagatcggaagagcgtcgtgt

SEQ ID NO: 1004 - DNA, 61 bp, synthetic construct
  source 1..61: mol_type = other DNA
  modified_base 1: mod_base = OTHER; note = 5' Biotin modified nucleotide with phosphorothioate linkage
  modified_base 2: mod_base = OTHER; note = Nucleotide with phosphorothioate linkage
  modified_base 3: mod_base = OTHER; note = Nucleotide with phosphorothioate linkage
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
  misc_difference 36: note = a, c, t, g, unknown or other
  misc_difference 37: note = a, c, t, g, unknown or other
  misc_difference 38: note = a, c, t, g, unknown or other
  misc_difference 39: note = a, c, t, g, unknown or other
  misc_difference 40: note = a, c, t, g, unknown or other
  misc_difference 41: note = a, c, t, g, unknown or other
  modified_base 58: mod_base = OTHER; note = Nucleotide with phosphorothioate linkage
  modified_base 59: mod_base = OTHER; note = Nucleotide with phosphorothioate linkage
  modified_base 60: mod_base = OTHER; note = Nucleotide with phosphorothioate linkage
agacgtgtgctcttccgatctgagggtacttnnnnnnnnnnagatcggaagagcgtcgtgt

SEQ ID NO: 1005 - DNA, 31 bp, synthetic construct
  source 1..31: mol_type = other DNA
actgagtgacttcacatggagcggtatggtt

SEQ ID NO: 1006 - DNA, 31 bp, synthetic construct
  source 1..31: mol_type = other DNA
tacggcctctaaacgacaatcggcactccaa

SEQ ID NO: 1007 - DNA, 35 bp, synthetic construct
  source 1..35: mol_type = other DNA
gaccataccggaagcaaactataatactgatattc

SEQ ID NO: 1008 - DNA, 32 bp, synthetic construct
  source 1..32: mol_type = other DNA
ccgtgaccaggtcattaccattgaacgagaat

SEQ ID NO: 1009 - DNA, 25 bp, synthetic construct
  source 1..25: mol_type = other DNA
acacctacgaaaaaacaaaattaat

SEQ ID NO: 1010 - DNA, 21 bp, synthetic construct
  source 1..21: mol_type = other DNA
attcagagcataaatagctat

SEQ ID NO: 1011 - DNA, 31 bp, synthetic construct
  source 1..31: mol_type = other DNA
taaccacagaaactttcatcagtatcttttc

SEQ ID NO: 1012 - DNA, 39 bp, synthetic construct
  source 1..39: mol_type = other DNA
gacggactcctataacccacaagaaacaaagtcctaatt

SEQ ID NO: 1013 - DNA, 35 bp, synthetic construct
  source 1..35: mol_type = other DNA
agcgaaaaatgccatcatctttgattgtaaaagtt

SEQ ID NO: 1014 - DNA, 35 bp, synthetic construct
  source 1..35: mol_type = other DNA
gaaccctaaggatatttaattcctgactatagcgt

SEQ ID NO: 1015 - DNA, 25 bp, synthetic construct
  source 1..25: mol_type = other DNA
ccgcgattaggacgcagtttgacgg

SEQ ID NO: 1016 - DNA, 39 bp, synthetic construct
  source 1..39: mol_type = other DNA
taaggtttagtcatagttcatgtagataccgaaggcttg

SEQ ID NO: 1017 - DNA, 35 bp, synthetic construct
  source 1..35: mol_type = other DNA
aatatttgaccctgaatcatacagaaaatccccct

SEQ ID NO: 1018 - DNA, 42 bp, synthetic construct
  source 1..42: mol_type = other DNA
ccaatacgcagacgacgataaaccaactagaaaccgattatc

SEQ ID NO: 1019 - DNA, 28 bp, synthetic construct
  source 1..28: mol_type = other DNA
caggctgaaagtacaaattcgtgaaagc

SEQ ID NO: 1020 - DNA, 25 bp, synthetic construct
  source 1..25: mol_type = other DNA
tacatccaagacgcgccccctcagc

SEQ ID NO: 1021 - DNA, 39 bp, synthetic construct
  source 1..39: mol_type = other DNA
aatacagtagggcaagccgaaacatggcaacaaaaaggg

SEQ ID NO: 1022 - DNA, 21 bp, synthetic construct
  source 1..21: mol_type = other DNA
attagcgaccagagccaccct

SEQ ID NO: 1023 - DNA, 25 bp, synthetic construct
  source 1..25: mol_type = other DNA
aagaaattttaatttatcgcgcatt

SEQ ID NO: 1024 - DNA, 35 bp, synthetic construct
  source 1..35: mol_type = other DNA
cagggaggagaaacacagtaacctaccagacaact

SEQ ID NO: 1025 - DNA, 24 bp, synthetic construct
  source 1..24: mol_type = other DNA
cccacctccttaccgagatttttt

SEQ ID NO: 1026 - DNA, 21 bp, synthetic construct
  source 1..21: mol_type = other DNA
caccagagccgcagcagaggg

SEQ ID NO: 1027 - DNA, 31 bp, synthetic construct
  source 1..31: mol_type = other DNA
ggccaaatcccagcgatcgggtaaaacaatt

SEQ ID NO: 1028 - DNA, 28 bp, synthetic construct
  source 1..28: mol_type = other DNA
tgcgggaacctaaaacgaattgatgatg

SEQ ID NO: 1029 - DNA, 31 bp, synthetic construct
  source 1..31: mol_type = other DNA
accctgagtttcacaaacaaaagaaagccca

SEQ ID NO: 1030 - DNA, 21 bp, synthetic construct
  source 1..21: mol_type = other DNA
gcattgacaggacaaattgta

SEQ ID NO: 1031 - DNA, 21 bp, synthetic construct
  source 1..21: mol_type = other DNA
gagcgggatcctgaaaagtgt

SEQ ID NO: 1032 - DNA, 39 bp, synthetic construct
  source 1..39: mol_type = other DNA
cattagttgatgccagctgtaatgattgccctagaatca

SEQ ID NO: 1033 - DNA, 25 bp, synthetic construct
  source 1..25: mol_type = other DNA
atatcgcatagtaattttttggatt

SEQ ID NO: 1034 - DNA, 25 bp, synthetic construct
  source 1..25: mol_type = other DNA
gcgtaaaatttaagggtgagaaagg

SEQ ID NO: 1035 - DNA, 35 bp, synthetic construct
  source 1..35: mol_type = other DNA
taatgttagcactagaaggttcatcacccaacagt

SEQ ID NO: 1036 - DNA, 31 bp, synthetic construct
  source 1..31: mol_type = other DNA
agagccattcggatgtttttgcccaatcaag

SEQ ID NO: 1037 - DNA, 21 bp, synthetic construct
  source 1..21: mol_type = other DNA
gctttgaaagagtcttatccg

SEQ ID NO: 1038 - DNA, 31 bp, synthetic construct
  source 1..31: mol_type = other DNA
cagtcgcgtcgcttgttggggtgcaacggta

SEQ ID NO: 1039 - DNA, 25 bp, synthetic construct
  source 1..25: mol_type = other DNA
gttatacaaaacataacgccaaaag

SEQ ID NO: 1040 - DNA, 27 bp, synthetic construct
  source 1..27: mol_type = other DNA
gaactggctcaacaaaaggtaaagtaa

SEQ ID NO: 1041 - DNA, 39 bp, synthetic construct
  source 1..39: mol_type = other DNA
aagcgggcgatcactccatcccaatgtataaggctatta

SEQ ID NO: 1042 - DNA, 38 bp, synthetic construct
  source 1..38: mol_type = other DNA
agatgatagcgcgatgtaaattcgagaaaggaacctta

SEQ ID NO: 1043 - DNA, 38 bp, synthetic construct
  source 1..38: mol_type = other DNA
cgtcaaatcattaagaaaccatcgatttattagcggcc

SEQ ID NO: 1044 - DNA, 54 bp, synthetic construct
  source 1..54: mol_type = other DNA
cagaggctttgaggacctttagattagcgaactgctattaaacgatgaataaca

SEQ ID NO: 1045 - DNA, 44 bp, synthetic construct
  source 1..44: mol_type = other DNA
ttacctcgagatttcgctcgctgaattcagaaagcggtgccgta

SEQ ID NO: 1046 - DNA, 45 bp, synthetic construct
  source 1..45: mol_type = other DNA
aaaagaaacatcggttaaaggtcttaaaagcctttgtttcaggcc

SEQ ID NO: 1047 - DNA, 38 bp, synthetic construct
  source 1..38: mol_type = other DNA
atacttcatacactagaaaacttatcataactacatca

SEQ ID NO: 1048 - DNA, 38 bp, synthetic construct
  source 1..38: mol_type = other DNA
cgacattgggtcgagaggtccacgctggcttctaatta

SEQ ID NO: 1049 - DNA, 39 bp, synthetic construct
  source 1..39: mol_type = other DNA
taagttgcaccgaaagcgtgtactggtaatatcaagagt

SEQ ID NO: 1050 - DNA, 42 bp, synthetic construct
  source 1..42: mol_type = other DNA
aaaaaaaggaaagagccaccaccaccggaaccgtcatagtta

SEQ ID NO: 1051 - DNA, 57 bp, synthetic construct
  source 1..57: mol_type = other DNA
tacatttaatacgtgacagcagtaggaatatagaaaacgctatacaaaaactgaaca

SEQ ID NO: 1052 - DNA, 51 bp, synthetic construct
  source 1..51: mol_type = other DNA
aaattatgggcgaagatggtgcagctgagtaaacataaagacccatcaaaa

SEQ ID NO: 1053 - DNA, 38 bp, synthetic construct
  source 1..38: mol_type = other DNA
tgccagtacgagcgttgatattcgtcacagagccattt

SEQ ID NO: 1054 - DNA, 40 bp, synthetic construct
  source 1..40: mol_type = other DNA
tacgagccgccccgggggttggtttccggcgcaactgaac

SEQ ID NO: 1055 - DNA, 52 bp, synthetic construct
  source 1..52: mol_type = other DNA
gtctttacggttgtaattagctgagagccagcataaacagttcaggcaatag

SEQ ID NO: 1056 - DNA, 38 bp, synthetic construct
  source 1..38: mol_type = other DNA
tttaaaggtggagcaataaagcctaccgcctgttgctg

SEQ ID NO: 1057 - DNA, 51 bp, synthetic construct
  source 1..51: mol_type = other DNA
ccctgattgagttactggcacaaacgtctcctcaccagggtaggctgcacc

SEQ ID NO: 1058 - DNA, 60 bp, synthetic construct
  source 1..60: mol_type = other DNA
ccggtattttttatgcactcagctgatggttaaataggacagcaaagcttgcggaaaatc

SEQ ID NO: 1059 - DNA, 51 bp, synthetic construct
  source 1..51: mol_type = other DNA
agttctggtgcaactctgtgagagagtataacgtgctttatcagtgcaaca

SEQ ID NO: 1060 - DNA, 56 bp, synthetic construct
  source 1..56: mol_type = other DNA
aataagaggcactgacctaatatatttagaggcaaaagatgaataatgcccgaacg

SEQ ID NO: 1061 - DNA, 38 bp, synthetic construct
  source 1..38: mol_type = other DNA
aaagcctacctcgagggcttaaagatcgcggtgcggtt

SEQ ID NO: 1062 - DNA, 49 bp, synthetic construct
  source 1..49: mol_type = other DNA
ctggacctacattttgacgctcattatagctgtttccccgacgcattct

SEQ ID NO: 1063 - DNA, 35 bp, synthetic construct
  source 1..35: mol_type = other DNA
gcgtccattacgagagggccagggtaaaggggcag

SEQ ID NO: 1064 - DNA, 56 bp, synthetic construct
  source 1..56: mol_type = other DNA
ccggagatcaacgctctgacccaatcgtctgaaatttaaatatgctcctccctcgt

SEQ ID NO: 1065 - DNA, 52 bp, synthetic construct
  source 1..52: mol_type = other DNA
cgccagaagctaaacgggcaagttccgaggccagtgggttttaaccgcctag

SEQ ID NO: 1066 - DNA, 35 bp, synthetic construct
  source 1..35: mol_type = other DNA
aataacaataaagagccgcaacagtatatcaaggt

SEQ ID NO: 1067 - DNA, 35 bp, synthetic construct
  source 1..35: mol_type = other DNA
gtaagaagagaagcaaccagaaatcaaatcgtcat

SEQ ID NO: 1068 - DNA, 42 bp, synthetic construct
  source 1..42: mol_type = other DNA
attctatcaaaattatttcgtcagagtttatcttacgagagc

SEQ ID NO: 1069 - DNA, 56 bp, synthetic construct
  source 1..56: mol_type = other DNA
ttcgagtctttcctttttcaaaatttagaccttcatcaaataatcctttggaaggg

SEQ ID NO: 1070 - DNA, 49 bp, synthetic construct
  source 1..49: mol_type = other DNA
gtacgcgctaggaagggaatgcgcattattctcaaagttttaacggaag

SEQ ID NO: 1071 - DNA, 56 bp, synthetic construct
  source 1..56: mol_type = other DNA
accatcaaaatcaccggatttgccagcgacaggaaacgtaccaggccgttgtatcg

SEQ ID NO: 1072 - DNA, 49 bp, synthetic construct
  source 1..49: mol_type = other DNA
agggattaaccaactatcggtgtgtgaaattgtgtccatcacgcaacgc

SEQ ID NO: 1073 - DNA, 49 bp, synthetic construct
  source 1..49: mol_type = other DNA
aaatagaggtctagctcaagtgcgggccttgagcaagtggcgccgctac

SEQ ID NO: 1074 - DNA, 41 bp, synthetic construct
  source 1..41: mol_type = other DNA
agagcattttccgtaacaaagtacctttcatctcggaacga

SEQ ID NO: 1075 - DNA, 35 bp, synthetic construct
  source 1..35: mol_type = other DNA
ctacacgcgaggcgttttaagacgctgagaagcta

SEQ ID NO: 1076 - DNA, 53 bp, synthetic construct
  source 1..53: mol_type = other DNA
aacctcaatgaaaggctttaatagtcagggctggcgtttcccaatcaaaagtc

SEQ ID NO: 1077 - DNA, 56 bp, synthetic construct
  source 1..56: mol_type = other DNA
cgtattactttaggtcatcttttttcgaccctcaggctcagtcaccaataggtgaa

SEQ ID NO: 1078 - DNA, 55 bp, synthetic construct
  source 1..55: mol_type = other DNA
tgagtaagtgtacagtgataacgccataccaccctgctgagaagaaaatttgagg

SEQ ID NO: 1079 - DNA, 56 bp, synthetic construct
  source 1..56: mol_type = other DNA
cactattaaagcgtcaccaaggccgaatcaagggattttagaatagaaggctccaa

SEQ ID NO: 1080 - DNA, 56 bp, synthetic construct
  source 1..56: mol_type = other DNA
aaattaatatcgaactcagttgtagcaatactgcttaattagcggtccggcgatta

SEQ ID NO: 1081 - DNA, 52 bp, synthetic construct
  source 1..52: mol_type = other DNA
cctgtagtcagaaatttgaccagctgaaatttcaaaattgagacaactaaca

SEQ ID NO: 1082 - DNA, 52 bp, synthetic construct
  source 1..52: mol_type = other DNA
ataatatcagagagtaagcagcaccgtaaaggttgaggcaggacgcctgacc

SEQ ID NO: 1083 - DNA, 38 bp, synthetic construct
  source 1..38: mol_type = other DNA
aaccaatttaaattatataacaacatccgaaacaccgg

SEQ ID NO: 1084 - DNA, 36 bp, synthetic construct
  source 1..36: mol_type = other DNA
aaacaaacatcaagggcaccatcgtcacaacttgct

SEQ ID NO: 1085 - DNA, 38 bp, synthetic construct
  source 1..38: mol_type = other DNA
tcatttgaattaccaagtttcgcaacggcaagccgtct

SEQ ID NO: 1086 - DNA, 49 bp, synthetic construct
  source 1..49: mol_type = other DNA
gagacaggaggccgattaaagggcttttttctcttataagtcacgagga

SEQ ID NO: 1087 - DNA, 60 bp, synthetic construct
  source 1..60: mol_type = other DNA
gatagcttaatatcagttattcagtagagtaccaaagcgctttattcagtcaaatcacca

SEQ ID NO: 1088 - DNA, 51 bp, synthetic construct
  source 1..51: mol_type = other DNA
taaaaacaatagcaggaaacgaaacgcgaacctacgacttattctaattac

SEQ ID NO: 1089 - DNA, 39 bp, synthetic construct
  source 1..39: mol_type = other DNA
ctatgtatcgggatttagagccccaaactgatagcccta

SEQ ID NO: 1090 - DNA, 58 bp, synthetic construct
  source 1..58: mol_type = other DNA
ataatgaagatttctgcgatctactaaattgggatgagtaatcttgattgcaaatgaa

SEQ ID NO: 1091 - DNA, 52 bp, synthetic construct
  source 1..52: mol_type = other DNA
ttatcacaacgtggaaaatcctttcacctggaccgtaatggggacgacatac

SEQ ID NO: 1092 - DNA, 39 bp, synthetic construct
  source 1..39: mol_type = other DNA
aaacacgttgataaccagctttgggaacaactcactcgt

SEQ ID NO: 1093 - DNA, 36 bp, synthetic construct
  source 1..36: mol_type = other DNA
aagcacaaagactataaaacgaggaacagaatgcag

SEQ ID NO: 1094 - DNA, 50 bp, synthetic construct
  source 1..50: mol_type = other DNA
gggtacattaaatataccaggcaattattttgcggaacaattgaaagaag

SEQ ID NO: 1095 - DNA, 35 bp, synthetic construct
  source 1..35: mol_type = other DNA
gtgtttttatacctcgtttcaccgccgaaaatgga

SEQ ID NO: 1096 - DNA, 35 bp, synthetic construct
  source 1..35: mol_type = other DNA
aacattattacctgacgaaatccaacaggtcagga

SEQ ID NO: 1097 - DNA, 37 bp, synthetic construct
  source 1..37: mol_type = other DNA
cagcgcctaaatcgagaaagggcgctgatcggctttg

SEQ ID NO: 1098 - DNA, 51 bp, synthetic construct
  source 1..51: mol_type = other DNA
agacggggagcaagaataacgtaaaggtgaaagtagtggttgccaaagcgc

SEQ ID NO: 1099 - DNA, 58 bp, synthetic construct
  source 1..58: mol_type = other DNA
caaatgccaagaaccagaacgctaacggttgggaatttctcacctgtttaaaccgtct

SEQ ID NO: 1100 - DNA, 50 bp, synthetic construct
  source 1..50: mol_type = other DNA
atcaagtaaataatgttagtgattaaattggcctctttccgcaagcaaat

SEQ ID NO: 1101 - DNA, 59 bp, synthetic construct
  source 1..59: mol_type = other DNA
caatagtgttatatgaatcatcgaactgaaaccaaagactggattatagtcagaagttc

SEQ ID NO: 1102 - DNA, 52 bp, synthetic construct
  source 1..52: mol_type = other DNA
ataataaagaattataaacagccatatttcctgaaatcctcacatgtacagg

SEQ ID NO: 1103 - DNA, 58 bp, synthetic construct
  source 1..58: mol_type = other DNA
ttaaatttgttaggtgtctggattaggaataagaacgtaaatgaacgcattatccatc

SEQ ID NO: 1104 - DNA, 52 bp, synthetic construct
  source 1..52: mol_type = other DNA
cagagcccggagtggctaaaccagccctaccgccagccagtagaagaaatgg

SEQ ID NO: 1105 - DNA, 45 bp, synthetic construct
  source 1..45: mol_type = other DNA
gccacgcaaaattacatcaatacgagtacctcaggagctacgagt

SEQ ID NO: 1106 - DNA, 42 bp, synthetic construct
  source 1..42: mol_type = other DNA
aagcacgcaataaacaatgaaatagcagggaaccaatccaaa

SEQ ID NO: 1107 - DNA, 60 bp, synthetic construct
  source 1..60: mol_type = other DNA
ctcatttaggcaaacgaaagtagtaatagtagggcaaagaccaaaaataaattaatgccg

SEQ ID NO: 1108 - DNA, 56 bp, synthetic construct
  source 1..56: mol_type = other DNA
cagtattaacaaaaatacgggcgcgattagataggggacataggtcagggtgggca

SEQ ID NO: 1109 - DNA, 51 bp, synthetic construct
  source 1..51: mol_type = other DNA
cgagtaacgagcacgttgcagcaagcaaaaatcatcatggtcaaatttttg

SEQ ID NO: 1110 - DNA, 25 bp, synthetic construct
  source 1..25: mol_type = other DNA
aaatattccaaatcgcttgccaggt

SEQ ID NO: 1111 - DNA, 28 bp, synthetic construct
  source 1..28: mol_type = other DNA
ttatgctggctatggtttcatgtaagaa

SEQ ID NO: 1112 - DNA, 26 bp, synthetic construct
  source 1..26: mol_type = other DNA
gagggaagtcacccacagcaggctgg

SEQ ID NO: 1113 - DNA, 28 bp, synthetic construct
  source 1..28: mol_type = other DNA
aatataatcctgaccccaatcgcattaa

SEQ ID NO: 1114 - DNA, 25 bp, synthetic construct
  source 1..25: mol_type = other DNA
gctttcattccgtaaacgcacagac

SEQ ID NO: 1115 - DNA, 28 bp, synthetic construct
  source 1..28: mol_type = other DNA
ccaaccacattatcataatttgataatc

SEQ ID NO: 1116 - DNA, 28 bp, synthetic construct
  source 1..28: mol_type = other DNA
aacaacgccaagaaatacgaacgcgaaa

SEQ ID NO: 1117 - DNA, 25 bp, synthetic construct
  source 1..25: mol_type = other DNA
gagaggggctaaatatgcgcgaaaa

SEQ ID NO: 1118 - DNA, 25 bp, synthetic construct
  source 1..25: mol_type = other DNA
tcaatattttgcggtacgtggttaa

SEQ ID NO: 1119 - DNA, 23 bp, synthetic construct
  source 1..23: mol_type = other DNA
cattcaagattctgagaatataa

SEQ ID NO: 1120 - DNA, 25 bp, synthetic construct
  source 1..25: mol_type = other DNA
gtaactgtatgtttgcctccccctt

SEQ ID NO: 1121 - DNA, 24 bp, synthetic construct
  source 1..24: mol_type = other DNA
ttagaacagtacctttagtttcaa

SEQ ID NO: 1122 - DNA, 28 bp, synthetic construct
  source 1..28: mol_type = other DNA
aaatctaaagatctaaacttgagaaatc

SEQ ID NO: 1123 - DNA, 25 bp, synthetic construct
  source 1..25: mol_type = other DNA
cgacacatgttggattatagagata

SEQ ID NO: 1124 - DNA, 25 bp, synthetic construct
  source 1..25: mol_type = other DNA
gccaacgttggataagaggttcagc

SEQ ID NO: 1125 - DNA, 21 bp, synthetic construct
  source 1..21: mol_type = other DNA
tcggtttatcagtaagcgcca

SEQ ID NO: 1126 - DNA, 28 bp, synthetic construct
  source 1..28: mol_type = other DNA
cagatcattacacgggtaagacaaacga

SEQ ID NO: 1127 - DNA, 25 bp, synthetic construct
  source 1..25: mol_type = other DNA
catttcggctggtgaattccgcttt

SEQ ID NO: 1128 - DNA, 25 bp, synthetic construct
  source 1..25: mol_type = other DNA
aaggcagcttgaaaccaaaacagaa

SEQ ID NO: 1129 - DNA, 21 bp, synthetic construct
  source 1..21: mol_type = other DNA
gaagagagaagcaccctccag

SEQ ID NO: 1130 - DNA, 21 bp, synthetic construct
  source 1..21: mol_type = other DNA
taattgagcgctaagaaccac

SEQ ID NO: 1131 - DNA, 21 bp, synthetic construct
  source 1..21: mol_type = other DNA
ttttgagagatctaattcgcc

SEQ ID NO: 1132 - DNA, 24 bp, synthetic construct
  source 1..24: mol_type = other DNA
gccagattttagacaggctaatga

SEQ ID NO: 1133 - DNA, 31 bp, synthetic construct
  source 1..31: mol_type = other DNA
aaccgttctagctgacattatttgaatgcaa

SEQ ID NO: 1134 - DNA, 28 bp, synthetic construct
  source 1..28: mol_type = other DNA
atataggaacgggaggatgaagcatgaa

SEQ ID NO: 1135 - DNA, 28 bp, synthetic construct
  source 1..28: mol_type = other DNA
aggtctttaccgagcttctttaattgca

SEQ ID NO: 1136 - DNA, 28 bp, synthetic construct
  source 1..28: mol_type = other DNA
ctcacaatcgtaatttctccgcggaaac

SEQ ID NO: 1137 - DNA, 25 bp, synthetic construct
  source 1..25: mol_type = other DNA
ttttttgcaaccgaacatacagaat

SEQ ID NO: 1138 - DNA, 42 bp, synthetic construct
  source 1..42: mol_type = other DNA
tttttgtaaccgtgcatctgcgatgtgctgcaaggcgttttt

SEQ ID NO: 1139 - DNA, 31 bp, synthetic construct
  source 1..31: mol_type = other DNA
tttttcccacgcataaccgatatattcgctg

SEQ ID NO: 1140 - DNA, 42 bp, synthetic construct
  source 1..42: mol_type = other DNA
tttttaggcggtttgcgtattccgagatagggttgagttttt

SEQ ID NO: 1141 - DNA, 42 bp, synthetic construct
  source 1..42: mol_type = other DNA
ttttttgttgttccagtttgggggaattagagccagcttttt

SEQ ID NO: 1142 - DNA, 42 bp, synthetic construct
  source 1..42: mol_type = other DNA
tttttttctgtccagacgacggataagtcctgaacaattttt

SEQ ID NO: 1143 - DNA, 42 bp, synthetic construct
  source 1..42: mol_type = other DNA
tttttcatgtcaatcatatgtgcaaatggtcaataacttttt

SEQ ID NO: 1144 - DNA, 32 bp, synthetic construct
  source 1..32: mol_type = other DNA
gagacaaaggctatcaggtcattgcctttttt

SEQ ID NO: 1145 - DNA, 42 bp, synthetic construct
  source 1..42: mol_type = other DNA
tttttctgtttagctatatttaattaccttatgcgatttttt

SEQ ID NO: 1146 - DNA, 32 bp, synthetic construct
  source 1..32: mol_type = other DNA
gccatttaacaagagaatagcgggcgccacgt

SEQ ID NO: 1147 - DNA, 28 bp, synthetic construct
  source 1..28: mol_type = other DNA
tttttacaaaatcgcgcagaggcgaatt

SEQ ID NO: 1148 - DNA, 38 bp, synthetic construct
  source 1..38: mol_type = other DNA
tggttctccgtcatcaacattaaatgtgagcgattttt

SEQ ID NO: 1149 - DNA, 42 bp, synthetic construct
  source 1..42: mol_type = other DNA
tttttattgcgtagattttcactttgaataccaagttttttt

SEQ ID NO: 1150 - DNA, 42 bp, synthetic construct
  source 1..42: mol_type = other DNA
tttttccagacgttagtaaataggaattgcgaataatttttt

SEQ ID NO: 1151 - DNA, 42 bp, synthetic construct
  source 1..42: mol_type = other DNA
ttttttttaagaactggctcaacaaaaggtaaagtaattttt

SEQ ID NO: 1152 - DNA, 42 bp, synthetic construct
  source 1..42: mol_type = other DNA
tttttttcatcggcattttcggcctccctcagagccgttttt

SEQ ID NO: 1153 - DNA, 46 bp, synthetic construct
  source 1..46: mol_type = other DNA
tttgacatttcaccccggatgaacggtaatcgtaaaactagttttt

SEQ ID NO: 1154 - DNA, 28 bp, synthetic construct
  source 1..28: mol_type = other DNA
tacccattgtgtcatttgcgaacgacaa

SEQ ID NO: 1155 - DNA, 42 bp, synthetic construct
  source 1..42: mol_type = other DNA
tttttgtaacaacccgtcggatgtagatgggcgcatcttttt

SEQ ID NO: 1156 - DNA, 42 bp, synthetic construct
  source 1..42: mol_type = other DNA
tttttgaaaaataatatcccaatgacaacaaccatcgttttt

SEQ ID NO: 1157 - DNA, 42 bp, synthetic construct
  source 1..42: mol_type = other DNA
tttttattaagttgggtaacgttgatataagtatagcttttt

SEQ ID NO: 1158 - DNA, 46 bp, synthetic construct
  source 1..46: mol_type = other DNA
ttaatgaatcgaaacctgattaattgcgttgcgctcactgcttttt

SEQ ID NO: 1159 - DNA, 42 bp, synthetic construct
  source 1..42: mol_type = other DNA
tttttccggaataggtgtatcaagttttgtcgtctttttttt

SEQ ID NO: 1160 - DNA, 25 bp, synthetic construct
  source 1..25: mol_type = other DNA
attgggtttaagcacgtaattagac

SEQ ID NO: 1161 - DNA, 42 bp, synthetic construct
  source 1..42: mol_type = other DNA
tttttccgctttccagtcggggccaacgcgcggggagttttt

SEQ ID NO: 1162 - DNA, 42 bp, synthetic construct
  source 1..42: mol_type = other DNA
tttttaaaatcaccagtagcacagactgtagcgcgttttttt

SEQ ID NO: 1163 - DNA, 42 bp, synthetic construct
  source 1..42: mol_type = other DNA
ttttttggtcagttggcaaattcaatagataatacatttttt

SEQ ID NO: 1164 - DNA, 42 bp, synthetic construct
  source 1..42: mol_type = other DNA
tttttttgaggatttagaagtaaacagaaataaagaattttt

SEQ ID NO: 1165 - DNA, 31 bp, synthetic construct
  source 1..31: mol_type = other DNA
attcattgattcgcgtcgctgtagttgccaa

SEQ ID NO: 1166 - DNA, 45 bp, synthetic construct
  source 1..45: mol_type = other DNA
tttttaattttttcacgttgaaaatctcgccgacatcctaataac

SEQ ID NO: 1167 - DNA, 42 bp, synthetic construct
  source 1..42: mol_type = other DNA
tttttgagagtctggagcaaaaccaccagcagaagatttttt

SEQ ID NO: 1168 - DNA, 28 bp, synthetic construct
  source 1..28: mol_type = other DNA
ttttcgatctaaccgtacagtaccgtta

SEQ ID NO: 1169 - DNA, 42 bp, synthetic construct
  source 1..42: mol_type = other DNA
tttttaaaacagaggtgaggcaccctcaatcaatatcttttt

SEQ ID NO: 1170 - DNA, 38 bp, synthetic construct
  source 1..38: mol_type = other DNA
tttttccaccctcagaaccgccaccctccaactaagaa

SEQ ID NO: 1171 - DNA, 40 bp, synthetic construct
  source 1..40: mol_type = other DNA
tttttttaattgctgaatattcaccagtcacacgattttt

SEQ ID NO: 1172 - DNA, 40 bp, synthetic construct
  source 1..40: mol_type = other DNA
ttttttatgcgttatacaaaacataacgccaaaagttttt

SEQ ID NO: 1173 - DNA, 40 bp, synthetic construct
  source 1..40: mol_type = other DNA
tttttgaagccttaaatcaaaccgttccagtaagcttttt

SEQ ID NO: 1174 - DNA, 40 bp, synthetic construct
  source 1..40: mol_type = other DNA
tttttgtcggtgggcacgaaatattaccgccagccttttt

SEQ ID NO: 1175 - DNA, 40 bp, synthetic construct
  source 1..40: mol_type = other DNA
ttttttaaccttgcttctgtcgcctgataaattgtttttt

SEQ ID NO: 1176 - DNA, 45 bp, synthetic construct
  source 1..45: mol_type = other DNA
agagagaggctaatcataaaccatgttacttagccggaacttttt

SEQ ID NO: 1177 - DNA, 41 bp, synthetic construct
  source 1..41: mol_type = other DNA
ccgaagcacagagatttttgtttaacgtcaaaaatgttttt

SEQ ID NO: 1178 - DNA, 37 bp, synthetic construct
  source 1..37: mol_type = other DNA
tttgattagtctcccgacttgcgggaggttttttttt

SEQ ID NO: 1179 - DNA, 21 bp, synthetic construct
  source 1..21: mol_type = other DNA
gaacggccaacttacatttgg

SEQ ID NO: 1180 - DNA, 40 bp, synthetic construct
  source 1..40: mol_type = other DNA
tttttgtcatacatggctttgtaacagtgcccgtattttt

SEQ ID NO: 1181 - DNA, 31 bp, synthetic construct
  source 1..31: mol_type = other DNA
tagcattaattaattttcccttagaattttt

SEQ ID NO: 1182 - DNA, 44 bp, synthetic construct
  source 1..44: mol_type = other DNA
cttgtatcataaatcgttaaatcaatatatgtgagtgaattttt

SEQ ID NO: 1183 - DNA, 40 bp, synthetic construct
  source 1..40: mol_type = other DNA
tttttattaagaggaagccctattttaaatgcaatttttt

SEQ ID NO: 1184 - DNA, 40 bp, synthetic construct
  source 1..40: mol_type = other DNA
tttttgaggcgcagacggtctttgcaaaagaagttttttt

SEQ ID NO: 1185 - DNA, 40 bp, synthetic construct
  source 1..40: mol_type = other DNA
tttttaaaatagcagcctttcctttttaagaaaagttttt

SEQ ID NO: 1186 - DNA, 40 bp, synthetic construct
  source 1..40: mol_type = other DNA
tttttctacctttttaacctgcctgtttagtatcattttt

SEQ ID NO: 1187 - DNA, 40 bp, synthetic construct
  source 1..40: mol_type = other DNA
ttttttaagcagatagccgaataagtttattttgtttttt

SEQ ID NO: 1188 - DNA, 45 bp, synthetic construct
  source 1..45: mol_type = other DNA
tttaggttgggaatttaacaggagcagtctctaccagatatctta

SEQ ID NO: 1189 - DNA, 40 bp, synthetic construct
  source 1..40: mol_type = other DNA
tttttgaattacgaggcatacggatggcttagagcttttt

SEQ ID NO: 1190 - DNA, 51 bp, synthetic construct
  source 1..51: mol_type = other DNA
tttttattgcaacaggaaaaacgctcaggcagataatgctgatttttggta

SEQ ID NO: 1191 - DNA, 35 bp, synthetic construct
  source 1..35: mol_type = other DNA
gtgcccctgccgccttgatgatgattcaaaatcga

SEQ ID NO: 1192 - DNA, 37 bp, synthetic construct
  source 1..37: mol_type = other DNA
tttttgcctgagtaatgtgtaggtaaagattcaatta

SEQ ID NO: 1193 - DNA, 36 bp, synthetic construct
  source 1..36: mol_type = other DNA
tgcccaaaatatgcagatttcttacagaaaaaccgg

SEQ ID NO: 1194 - DNA, 38 bp, synthetic construct
  source 1..38: mol_type = other DNA
actcaactaaaccccgcagtgtccacgtggcggaaccc

SEQ ID NO: 1195 - DNA, 38 bp, synthetic construct
  source 1..38: mol_type = other DNA
tttttccagtaataaaagggacattctcctcatagaaa

SEQ ID NO: 1196 - DNA, 32 bp, synthetic construct
  source 1..32: mol_type = other DNA
taaagggattcataccacggaacaaagttgaa

SEQ ID NO: 1197 - DNA, 40 bp, synthetic construct
  source 1..40: mol_type = other DNA
tttttcaccacacccgccgctctttgattagtaatttttt

SEQ ID NO: 1198 - DNA, 44 bp, synthetic construct
  source 1..44: mol_type = other DNA
tttttaacatcacttgcctgagtagaacagaacatatagggccc

SEQ ID NO: 1199 - DNA, 40 bp, synthetic construct
  source 1..40: mol_type = other DNA
tttttttgccagagggggtagattgcatcaaaaagttttt

SEQ ID NO: 1200 - DNA, 40 bp, synthetic construct
  source 1..40: mol_type = other DNA
tttttatgacaatgtcccgcatctgtaagcaactcttttt

SEQ ID NO: 1201 - DNA, 40 bp, synthetic construct
  source 1..40: mol_type = other DNA
ttttttaaacagttaatgcctgaattgtcaaccttttttt

SEQ ID NO: 1202 - DNA, 40 bp, synthetic construct
  source 1..40: mol_type = other DNA
tttttcacaatcaatagaaaagcccccgatttagattttt

SEQ ID NO: 1203 - DNA, 40 bp, synthetic construct
  source 1..40: mol_type = other DNA
ttttttccttgaaaacatagcataggtctgagagattttt

SEQ ID NO: 1204 - DNA, 35 bp, synthetic construct
  source 1..35: mol_type = other DNA
gaccaaagcgatagtaaaatgtttaatagcgcaac

SEQ ID NO: 1205 - DNA, 40 bp, synthetic construct
  source 1..40: mol_type = other DNA
tttttgcttgacggggaaagcacgctgcgcgtaacttttt

SEQ ID NO: 1206 - DNA, 38 bp, synthetic construct
  source 1..38: mol_type = other DNA
tttttgtcgaaatccgcgacctgctttatcatcagaga

SEQ ID NO: 1207 - DNA, 44 bp, synthetic construct
  source 1..44: mol_type = other DNA
acccaaataatcttaccggcttatacacgacgctcttccgatct

SEQ ID NO: 1208 - DNA, 51 bp, synthetic construct
  source 1..51: mol_type = other DNA
tattcagctcctcgaatttccacaaggccacacacgacgctcttccgatct

SEQ ID NO: 1209 - DNA, 55 bp, synthetic construct
  source 1..55: mol_type = other DNA
cgcgccttgaatataataacgtcaattacctgagcacacgacgctcttccgatct

SEQ ID NO: 1210 - DNA, 48 bp, synthetic construct
  source 1..48: mol_type = other DNA
gtgagctaaacggcggattgttggccttacacgacgctcttccgatct